SEQUENCE LISTING (26 sequences)

SEQ ID NO: 1 - protein, 282 aa, unidentified
MNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEE

SEQ ID NO: 2 - protein, 12 aa, synthetic construct
SGSGSGSGSGSG

SEQ ID NO: 3 - protein, 238 aa, synthetic construct
MSKGEELFTGVVPILVELDGDVNGHKFSVSGEGEGDATYGKLTLKFICTTGKLPVPWPTLVTTFSYGVQCFARYPDHMKQHDFFKSAMPEGYVQERTIFFKDDGNYKTRAEVKFEGDTLVNRIELKGIDFKEDGNILGHKLEYNFNSHNVYIMADKQKNGIKVNFKIRHNIEDGSVQLADHYQQNTPIGDGPVLLPDNHYLSIQSALSKDPNEKRDHMVLLEFVTAAGITHGIDELYK

SEQ ID NO: 4 - protein, 12 aa, synthetic construct
GGSGGSGSGGSG

SEQ ID NO: 5 - protein, 238 aa, synthetic construct
MRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITHGMDELYK

SEQ ID NO: 6 - protein, 8 aa, synthetic construct
DYKDDDDK

SEQ ID NO: 7 - protein, 347 aa, synthetic construct
MSRLSINPTLINRDKPYTKEELMEILRLAIIAELDAINLYEQMARYSEDENVRKILLDVAREEKAHVGEFMALLLNLDPEQVTELKGGFEEVKELTGIEAHINDNKKEESNVEYFEKLRSALLDGVNKGRSLLKHLPVTRIEGQSFRVDIIKFEDGVRVVKQEYKPIPLLKKKFYVGIRELNDGTYDVSIATKAGELLVKDEESLVIREILSTEGIKKMKLSSWDNPEEALNDLMNALQEASNASAGPFGLIINPKRYAKLLKIYEKSGKMLVEVLKEIFRGGIIVTLNIDENKVIIFANTPAVLDVVVGQDVTLQELGPEGDDVAFLVSEAIGIRIKNPEAIVVLE

SEQ ID NO: 8 - protein, 297 aa, synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 9 - protein, 78 aa, synthetic construct
MSEVAEQLVGVSAASAAASAFGSLSSALLMPKDGRTLEDVVRELLRPLLKEWLDQNLPRIVETKVEEEVQRISRGRGA

SEQ ID NO: 10 - protein, 232 aa, synthetic construct
MVSKGEAVIKEFMRFKVHMEGSMNGHEFEIEGEGEGRPYEGTQTAKLKVTKGGPLPFSWDILSPQFMYGSRAFIKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYK

SEQ ID NO: 11 - protein, 616 aa, synthetic construct
MSEVAEQLVGVSAASAAASAFGSLSSALLMPKDGRTLEDVVRELLRPLLKEWLDQNLPRIVETKVEEEVQRISRGRGASRMNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEWSGSGSGSGSGSGRPLMSKGEELFTGVVPILVELDGDVNGHKFSVSGEGEGDATYGKLTLKFICTTGKLPVPWPTLVTTFSYGVQCFARYPDHMKQHDFFKSAMPEGYVQERTIFFKDDGNYKTRAEVKFEGDTLVNRIELKGIDFKEDGNILGHKLEYNFNSHNVYIMADKQKNGIKVNFKIRHNIEDGSVQLADHYQQNTPIGDGPVLLPDNHYLSIQSALSKDPNEKRDHMVLLEFVTAAGITHGIDELYK

SEQ ID NO: 12 - protein, 675 aa, synthetic construct
MSEVAEQLVGVSAASAAASAFGSLSSALLMPKDGRTLEDVVRELLRPLLKEWLDQNLPRIVETKVEEEVQRISRGRGASRMNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEWSGSGSGSGSGSGRPLMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 13 - protein, 610 aa, synthetic construct
MSEVAEQLVGVSAASAAASAFGSLSSALLMPKDGRTLEDVVRELLRPLLKEWLDQNLPRIVETKVEEEVQRISRGRGASRMNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEWSGSGSGSGSGSGRPLMVSKGEAVIKEFMRFKVHMEGSMNGHEFEIEGEGEGRPYEGTQTAKLKVTKGGPLPFSWDILSPQFMYGSRAFIKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYK

SEQ ID NO: 14 - protein, 541 aa, synthetic construct
MNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEWSGSGSGSGSGSGRRIPGLIKMSKGEELFTGVVPILVELDGDVNGHKFSVSGEGEGDATYGKLTLKFICTTGKLPVPWPTLVTTFSYGVQCFARYPDHMKQHDFFKSAMPEGYVQERTIFFKDDGNYKTRAEVKFEGDTLVNRIELKGIDFKEDGNILGHKLEYNFNSHNVYIMADKQKNGIKVNFKIRHNIEDGSVQLADHYQQNTPIGDGPVLLPDNHYLSIQSALSKDPNEKRDHMVLLEFVTAAGITHGIDELYK

SEQ ID NO: 15 - protein, 552 aa, synthetic construct
MNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGGSGGSGSGGSGMRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITHGMDELYKGGSGGSGSGGSGDYKDDDDK

SEQ ID NO: 16 - DNA, 1626 bp, synthetic construct
  source 1..1626: mol_type = other DNA
atgaacaaaagccaactttatccggattcaccactgacggatcaggacttcaaccaattagaccaaaccgtgattgaggctgctcgtcgtcagctggtgggtcgtcgcttcattgagttatatggcccattggggcgtggcatgcagagtgtcttcaacgatatcttcatggagtctcatgaagcgaaaatggacttccagggcagctttgacacggaggtagagtcctcccgtcgtgtaaactataccattccgatgttatataaagacttcgtgctttactggcgcgatctggaacagagcaaggcactcgatattccgatcgacttttcagtggcagcgaacgctgcccgcgacgttgcgttcctggaagatcagatgattttccatggaagcaaagaatttgatatcccgggtctgatgaacgtgaaaggtcgcctgacccatctgattggcaattggtatgagtcgggtaacgcctttcaggatattgtggaggcccgcaataaattactcgaaatgaaccacaatggcccatatgctctcgtgctgtccccggagctgtactcactcttacatcgtgtgcataaagacacgaatgtgctggagatcgaacacgtgcgcgagttgattactgctggggtttttcagtcgcctgtcctcaaagggaaaagtggtgtgatcgtaaacaccggtcgcaacaatctggatttggctatctcggaagattttgagactgcatacctgggcgaggaaggtatgaaccatccctttcgcgtgtacgagacagttgttctgcgcatcaaacgcccggcggccatttgtactttaatcgatccggaagaatggtctggcagcggtagtgggtcgggatcaggctccggtcgacggatccccgggttaattaagatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttttcttatggtgttcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactttaactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccattcaatctgccttatccaaagatccaaacgaaaagagagaccacatggtcttgttagaatttgttactgctgctggtattacccatggtattgatgaattgtacaaataa

SEQ ID NO: 17 - DNA, 1851 bp, synthetic construct
  source 1..1851: mol_type = other DNA
atgtccgaggtcgcagagcagctggttggcgtctctgccgcctctgcggctgcaagcgcctttggctcactgtcaagcgccttattaatgcccaaagacggcagaacactggaggacgtagtgagagagctgctgcggccactgttgaaggagtggctagaccagaacctccctcgcattgtagagaccaaggttgaagaggaagtgcagcggatcagtagaggtcggggcgcatctagaatgaataagtctcagctgtacccagacagtcccctcaccgaccaagattttaatcagttggatcagacagttatcgaagcagcacggcgccaattggtcggccgacgatttatcgaactgtacggacccctcggacgcgggatgcaaagcgtgtttaatgacattttcatggaaagtcacgaggctaagatggattttcaagggtccttcgataccgaagtggaatcttcaagacgcgtcaattacacaatccctatgctgtacaaggattttgttctgtattggcgggaccttgagcaatctaaagcgctggacatccccatagatttcagtgtagctgctaatgccgctagggatgtggcctttcttgaggaccaaatgatctttcacggctcaaaggagttcgacattcctgggttaatgaatgtcaaggggcggctcacacacctcatcggaaactggtacgaatccggcaatgctttccaagacatcgtcgaagcaaggaacaagttgttagagatgaatcataacgggccgtacgccctggttttgtcaccagaactttatagcctgctccaccgggtacacaaggatactaacgttctcgaaattgagcatgtcagggaactgataaccgccggtgtattccaaagccccgtactgaagggaaagtccggagtcattgtgaatacgggacggaataacctcgacctcgcaataagcgaggacttcgaaaccgcttatttgggagaagagggcatgaatcacccattcagggtctatgaaaccgtggtcttgagaataaagaggccagccgcgatctgcaccctcattgaccctgaggagtggtcagggtcaggttctggctcaggtagcgggagtgggcggccgctgatgtctaagggggaggagcttttcacaggtgtagtacctattcttgttgaattggatggcgatgtcaatggacacaaattctccgtctcaggggagggagagggagacgctacctatggaaagttgactctcaaattcatctgcacaacgggtaagttgccggtcccttggccgacactcgttactacgttcagctatggagtacagtgtttcgcgcgctaccccgaccatatgaaacaacacgactttttcaaatccgcaatgcccgagggatacgttcaggaacggactattttctttaaagacgacggtaattacaaaacacgcgccgaagttaagttcgaaggagatactttggttaaccgaatagagctgaaaggcattgatttcaaagaggacgggaacatcctgggtcataaacttgagtataattttaattcccacaacgtgtatataatggcagataaacagaagaatggcatcaaggttaacttcaagattcggcacaatattgaagacggtagtgtacagctggctgaccactatcagcagaatacgccaattggtgatgggcccgtcttgttgccagataatcactacctctcaatccagagtgcccttagtaaagatccaaacgaaaagcgggaccacatggttttgttggagtttgtcacagctgctgggattacccacgggatagacgagttgtataaatga

SEQ ID NO: 18 - DNA, 2028 bp, synthetic construct
  source 1..2028: mol_type = other DNA
atgtccgaggtcgcagagcagctggttggcgtctctgccgcctctgcggctgcaagcgcctttggctcactgtcaagcgccttattaatgcccaaagacggcagaacactggaggacgtagtgagagagctgctgcggccactgttgaaggagtggctagaccagaacctccctcgcattgtagagaccaaggttgaagaggaagtgcagcggatcagtagaggtcggggcgcatctagaatgaataagtctcagctgtacccagacagtcccctcaccgaccaagattttaatcagttggatcagacagttatcgaagcagcacggcgccaattggtcggccgacgatttatcgaactgtacggacccctcggacgcgggatgcaaagcgtgtttaatgacattttcatggaaagtcacgaggctaagatggattttcaagggtccttcgataccgaagtggaatcttcaagacgcgtcaattacacaatccctatgctgtacaaggattttgttctgtattggcgggaccttgagcaatctaaagcgctggacatccccatagatttcagtgtagctgctaatgccgctagggatgtggcctttcttgaggaccaaatgatctttcacggctcaaaggagttcgacattcctgggttaatgaatgtcaaggggcggctcacacacctcatcggaaactggtacgaatccggcaatgctttccaagacatcgtcgaagcaaggaacaagttgttagagatgaatcataacgggccgtacgccctggttttgtcaccagaactttatagcctgctccaccgggtacacaaggatactaacgttctcgaaattgagcatgtcagggaactgataaccgccggtgtattccaaagccccgtactgaagggaaagtccggagtcattgtgaatacgggacggaataacctcgacctcgcaataagcgaggacttcgaaaccgcttatttgggagaagagggcatgaatcacccattcagggtctatgaaaccgtggtcttgagaataaagaggccagccgcgatctgcaccctcattgaccctgaggagtggtcagggtcaggttctggctcaggtagcgggagtgggcggccgctgatggcagaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctcgagatttccggctag

SEQ ID NO: 19 - DNA, 1833 bp, synthetic construct
  source 1..1833: mol_type = other DNA
atgtccgaggtcgcagagcagctggttggcgtctctgccgcctctgcggctgcaagcgcctttggctcactgtcaagcgccttattaatgcccaaagacggcagaacactggaggacgtagtgagagagctgctgcggccactgttgaaggagtggctagaccagaacctccctcgcattgtagagaccaaggttgaagaggaagtgcagcggatcagtagaggtcggggcgcatctagaatgaataagtctcagctgtacccagacagtcccctcaccgaccaagattttaatcagttggatcagacagttatcgaagcagcacggcgccaattggtcggccgacgatttatcgaactgtacggacccctcggacgcgggatgcaaagcgtgtttaatgacattttcatggaaagtcacgaggctaagatggattttcaagggtccttcgataccgaagtggaatcttcaagacgcgtcaattacacaatccctatgctgtacaaggattttgttctgtattggcgggaccttgagcaatctaaagcgctggacatccccatagatttcagtgtagctgctaatgccgctagggatgtggcctttcttgaggaccaaatgatctttcacggctcaaaggagttcgacattcctgggttaatgaatgtcaaggggcggctcacacacctcatcggaaactggtacgaatccggcaatgctttccaagacatcgtcgaagcaaggaacaagttgttagagatgaatcataacgggccgtacgccctggttttgtcaccagaactttatagcctgctccaccgggtacacaaggatactaacgttctcgaaattgagcatgtcagggaactgataaccgccggtgtattccaaagccccgtactgaagggaaagtccggagtcattgtgaatacgggacggaataacctcgacctcgcaataagcgaggacttcgaaaccgcttatttgggagaagagggcatgaatcacccattcagggtctatgaaaccgtggtcttgagaataaagaggccagccgcgatctgcaccctcattgaccctgaggagtggtcagggtcaggttctggctcaggtagcgggagtgggcggccgctgatggttagtaaaggtgaagctgttataaaagaattcatgaggtttaaagttcatatggaaggttcaatgaatggtcatgaatttgaaattgaaggtgaaggtgaaggtagaccatatgaaggtacacaaactgctaaattgaaagttactaaaggtggtccattaccattttcttgggatattttgtctccacaattcatgtatggttctagagcttttattaagcatccagctgatattccagattattataaacaatcttttcctgaaggttttaaatgggaaagagttatgaatttcgaagatggtggtgctgttactgttactcaagatacttctttggaagatggtactttaatctataaagttaaattgagaggtactaattttccaccagatggtccagttatgcaaaagaaaactatgggttgggaagcatctactgaaagattgtatccagaagatggtgttttgaaaggagacattaagatggctttgagattgaaagatggtggtcggtacctggccgacttcaagaccacctataaagctaaaaaaccagttcaaatgccaggtgcatataatgttgatagaaagttagatataacgtcgcataacgaggactatactgtagttgaacaatatgaacgtagtgaaggtagacatagtaccggaggaatggatgaattgtataaataa

SEQ ID NO: 20 - DNA, 1659 bp, synthetic construct
  source 1..1659: mol_type = other DNA
atgaacaaaagccaactttatccggattcaccactgacggatcaggacttcaaccaattagaccaaaccgtgattgaggctgctcgtcgtcagctggtgggtcgtcgcttcattgagttatatggcccattggggcgtggcatgcagagtgtcttcaacgatatcttcatggagtctcatgaagcgaaaatggacttccagggcagctttgacacggaggtagagtcctcccgtcgtgtaaactataccattccgatgttatataaagacttcgtgctttactggcgcgatctggaacagagcaaggcactcgatattccgatcgacttttcagtggcagcgaacgctgcccgcgacgttgcgttcctggaagatcagatgattttccatggaagcaaagaatttgatatcccgggtctgatgaacgtgaaaggtcgcctgacccatctgattggcaattggtatgagtcgggtaacgcctttcaggatattgtggaggcccgcaataaattactcgaaatgaaccacaatggcccatatgctctcgtgctgtccccggagctgtactcactcttacatcgtgtgcataaagacacgaatgtgctggagatcgaacacgtgcgcgagttgattactgctggggtttttcagtcgcctgtcctcaaagggaaaagtggtgtgatcgtaaacaccggtcgcaacaatctggatttggctatctcggaagattttgagactgcatacctgggcgaggaaggtatgaaccatccctttcgcgtgtacgagacagttgttctgcgcatcaaacgcccggcggccatttgtactttaatcgatccggaagaaggaggttctggtggaagtggttccgggggaagcggtatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggttccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcaaactgtctaaagatccgaacgagaaacgcgatcatatggttctgctggagttcgtaaccgcagcgggcatcacgcatggtatggatgaactgtacaaaggaggttctggtggaagtggttccgggggaagcggtgactacaaagacgatgacgacaagtag

SEQ ID NO: 21 - DNA, 1815 bp, synthetic construct
  source 1..1815: mol_type = other DNA
atgctctcaataaatccaacccttataaatagggataagccatacaccaaagaagaattaatggaaattctgagattagcaataatagctgagcttgatgcaataaatctatatgagcagatggcaagatattccgaagatgaaaacgtaagaaagattctccttgatgttgcaagggaggagaaggctcacgttggagaattcatggcactccttctaaatttggatccagaacaagtaactgagctgaagggtggatttgaggaagtcaaggagctaactggaattgaggctcacataaatgataacaagaaggaggaatctaacgtggaatattttgagaagttgagaagtgctcttttggatggggtaaataagggaagatctttactaaagcacttgccagtaacgagaatagaaggacaatcattcagggttgacatcattaaatttgaagatggtgttagggtagtaaagcaagagtacaagccaattccattattgaagaagaaattctatgtgggaataagagaactcaacgatgggacttacgatgtatctatagcaactaaagctggggaactcctagttaaagacgaagagtcgctagtaataagagaaattctatccacagagggaattaagaaaatgaaactttcctcctgggacaatccagaagaagcacttaatgatttaatgaacgcactgcaggaagcctcaaatgcgtcagctggaccatttgggctgataattaacccaaaaagatatgcaaagttgctaaaaatttacgagaagagtggaaagatgctagttgaagtgctaaaagagatattcaggggaggaatcattgttactctaaacattgatgagaataaggtaataatatttgccaatacaccagctgttcttgacgttgtagttggccaagatgtgactctacaagagctaggacctgaaggagacgacgttgcattcttggttagtgaagctataggaataagaataaagaacccagaggcaatagtagtcttagagtggtctggcagcggtagtgggtcgggatcaggctccggtcgacggatccccgggttaattaagatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttttcttatggtgttcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactttaactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccattcaatctgccttatccaaagatccaaacgaaaagagagaccacatggtcttgttagaatttgttactgctgctggtattacccatggtattgatgaattgtacaaataa

SEQ ID NO: 22 - DNA, 1875 bp, synthetic construct
  source 1..1875: mol_type = other DNA
atgctctcaataaatccaacccttataaatagggataagccatacaccaaagaagaattaatggaaattctgagattagcaataatagctgagcttgatgcaataaatctatatgagcagatggcaagatattccgaagatgaaaacgtaagaaagattctccttgatgttgcaagggaggagaaggctcacgttggagaattcatggcactccttctaaatttggatccagaacaagtaactgagctgaagggtggatttgaggaagtcaaggagctaactggaattgaggctcacataaatgataacaagaaggaggaatctaacgtggaatattttgagaagttgagaagtgctcttttggatggggtaaataagggaagatctttactaaagcacttgccagtaacgagaatagaaggacaatcattcagggttgacatcattaaatttgaagatggtgttagggtagtaaagcaagagtacaagccaattccattattgaagaagaaattctatgtgggaataagagaactcaacgatgggacttacgatgtatctatagcaactaaagctggggaactcctagttaaagacgaagagtcgctagtaataagagaaattctatccacagagggaattaagaaaatgaaactttcctcctgggacaatccagaagaagcacttaatgatttaatgaacgcactgcaggaagcctcaaatgcgtcagctggaccatttgggctgataattaacccaaaaagatatgcaaagttgctaaaaatttacgagaagagtggaaagatgctagttgaagtgctaaaagagatattcaggggaggaatcattgttactctaaacattgatgagaataaggtaataatatttgccaatacaccagctgttcttgacgttgtagttggccaagatgtgactctacaagagctaggacctgaaggagacgacgttgcattcttggttagtgaagctataggaataagaataaagaacccagaggcaatagtagtcttagagtggtctggcagcggtagtgggtcgggatcaggctccggtcgacggatccccgggttaattaagatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttttcttatggtgttcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactttaactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccattcaatctgccttatccaaagatccaaacgaaaagagagaccacatggtcttgttagaatttgttactgctgctggtattacccatggtattgatgaattgtacaaaggaggttctggtggaagtggttccgggggaagcggtgactacaaagacgatgacgacaagtaa

SEQ ID NO: 23 - protein, 604 aa, synthetic construct
MLSINPTLINRDKPYTKEELMEILRLAIIAELDAINLYEQMARYSEDENVRKILLDVAREEKAHVGEFMALLLNLDPEQVTELKGGFEEVKELTGIEAHINDNKKEESNVEYFEKLRSALLDGVNKGRSLLKHLPVTRIEGQSFRVDIIKFEDGVRVVKQEYKPIPLLKKKFYVGIRELNDGTYDVSIATKAGELLVKDEESLVIREILSTEGIKKMKLSSWDNPEEALNDLMNALQEASNASAGPFGLIINPKRYAKLLKIYEKSGKMLVEVLKEIFRGGIIVTLNIDENKVIIFANTPAVLDVVVGQDVTLQELGPEGDDVAFLVSEAIGIRIKNPEAIVVLEWSGSGSGSGSGSGRRIPGLIKMSKGEELFTGVVPILVELDGDVNGHKFSVSGEGEGDATYGKLTLKFICTTGKLPVPWPTLVTTFSYGVQCFARYPDHMKQHDFFKSAMPEGYVQERTIFFKDDGNYKTRAEVKFEGDTLVNRIELKGIDFKEDGNILGHKLEYNFNSHNVYIMADKQKNGIKVNFKIRHNIEDGSVQLADHYQQNTPIGDGPVLLPDNHYLSIQSALSKDPNEKRDHMVLLEFVTAAGITHGIDELYK

SEQ ID NO: 24 - protein, 624 aa, synthetic construct
MLSINPTLINRDKPYTKEELMEILRLAIIAELDAINLYEQMARYSEDENVRKILLDVAREEKAHVGEFMALLLNLDPEQVTELKGGFEEVKELTGIEAHINDNKKEESNVEYFEKLRSALLDGVNKGRSLLKHLPVTRIEGQSFRVDIIKFEDGVRVVKQEYKPIPLLKKKFYVGIRELNDGTYDVSIATKAGELLVKDEESLVIREILSTEGIKKMKLSSWDNPEEALNDLMNALQEASNASAGPFGLIINPKRYAKLLKIYEKSGKMLVEVLKEIFRGGIIVTLNIDENKVIIFANTPAVLDVVVGQDVTLQELGPEGDDVAFLVSEAIGIRIKNPEAIVVLEWSGSGSGSGSGSGRRIPGLIKMSKGEELFTGVVPILVELDGDVNGHKFSVSGEGEGDATYGKLTLKFICTTGKLPVPWPTLVTTFSYGVQCFARYPDHMKQHDFFKSAMPEGYVQERTIFFKDDGNYKTRAEVKFEGDTLVNRIELKGIDFKEDGNILGHKLEYNFNSHNVYIMADKQKNGIKVNFKIRHNIEDGSVQLADHYQQNTPIGDGPVLLPDNHYLSIQSALSKDPNEKRDHMVLLEFVTAAGITHGIDELYKGGSGGSGSGGSGDYKDDDDK

SEQ ID NO: 25 - DNA, 1806 bp, synthetic construct
  source 1..1806: mol_type = other DNA
atgtctagactctctatcaacccgacattgataaaccgggacaaaccgtatactaaagaagaacttatggaaatcctgcggctggcaatcattgctgagttggacgcgatcaacctctatgaacagatggcacggtattctgaagatgagaacgtaaggaagattctccttgacgtcgctcgggaggagaaggcgcacgtgggtgagtttatggcactccttcttaaccttgaccctgagcaagtgactgagttgaaaggcggcttcgaagaggtaaaagaactgacaggcattgaagcccatattaacgataataaaaaagaagagtccaacgtcgaatattttgaaaaactccgctccgctctgttggatggagtaaataaggggaggagtctccttaagcatctgccagttacacgcatagaagggcagtcctttcgagtagacattattaaatttgaggatggggtgcgcgtcgttaaacaggaatacaagcccattccacttctcaaaaagaaattttacgtaggaattagagaactgaatgatggtacgtacgacgtttctattgccacgaaggcaggtgaattgttggttaaggacgaggaaagccttgtgataagagaaatcctgtcaactgaagggataaagaaaatgaagttgagtagctgggacaatcccgaggaggcgctgaatgatcttatgaatgcccttcaggaagcctctaatgcttccgctggacctttcggtctgataattaatccaaagagatacgcgaaacttcttaaaatctatgagaaatcaggaaaaatgttggtagaagtgctgaaagaaatttttcggggtggcatcatagtcacactgaacatagatgaaaacaaggttattatttttgcaaatactccagcggtgcttgacgtagttgttgggcaggacgtaacgctccaggaattgggacctgagggggacgacgtggcctttttggtgtcagaagccatcggcatccgaataaagaaccccgaagcgattgttgtcctcgaatggtcagggtcaggttctggctcaggtagcgggagtgggcggccgctgatgtctaagggggaggagcttttcacaggtgtagtacctattcttgttgaattggatggcgatgtcaatggacacaaattctccgtctcaggggagggagagggagacgctacctatggaaagttgactctcaaattcatctgcacaacgggtaagttgccggtcccttggccgacactcgttactacgttcagctatggagtacagtgtttcgcgcgctaccccgaccatatgaaacaacacgactttttcaaatccgcaatgcccgagggatacgttcaggaacggactattttctttaaagacgacggtaattacaaaacacgcgccgaagttaagttcgaaggagatactttggttaaccgaatagagctgaaaggcattgatttcaaagaggacgggaacatcctgggtcataaacttgagtataattttaattcccacaacgtgtatataatggcagataaacagaagaatggcatcaaggttaacttcaagattcggcacaatattgaagacggtagtgtacagctggctgaccactatcagcagaatacgccaattggtgatgggcccgtcttgttgccagataatcactacctctcaatccagagtgcccttagtaaagatccaaacgaaaagcgggaccacatggttttgttggagtttgtcacagctgctgggattacccacgggatagacgagttgtataaatga

SEQ ID NO: 26 - protein, 601 aa, synthetic construct
MSRLSINPTLINRDKPYTKEELMEILRLAIIAELDAINLYEQMARYSEDENVRKILLDVAREEKAHVGEFMALLLNLDPEQVTELKGGFEEVKELTGIEAHINDNKKEESNVEYFEKLRSALLDGVNKGRSLLKHLPVTRIEGQSFRVDIIKFEDGVRVVKQEYKPIPLLKKKFYVGIRELNDGTYDVSIATKAGELLVKDEESLVIREILSTEGIKKMKLSSWDNPEEALNDLMNALQEASNASAGPFGLIINPKRYAKLLKIYEKSGKMLVEVLKEIFRGGIIVTLNIDENKVIIFANTPAVLDVVVGQDVTLQELGPEGDDVAFLVSEAIGIRIKNPEAIVVLEWSGSGSGSGSGSGRPLMSKGEELFTGVVPILVELDGDVNGHKFSVSGEGEGDATYGKLTLKFICTTGKLPVPWPTLVTTFSYGVQCFARYPDHMKQHDFFKSAMPEGYVQERTIFFKDDGNYKTRAEVKFEGDTLVNRIELKGIDFKEDGNILGHKLEYNFNSHNVYIMADKQKNGIKVNFKIRHNIEDGSVQLADHYQQNTPIGDGPVLLPDNHYLSIQSALSKDPNEKRDHMVLLEFVTAAGITHGIDELYK

(19) United States
(12) Patent Application Publication
(10) Pub. No.: US 2026/0009731 A1
(43) Pub. Date: Jan. 8, 2026

(54) GENETICALLY ENCODED MULTIMERIC NANOPARTICLES FOR PHYSICAL ANALYSIS OF THE CELL INTERIOR

(71) Applicant: NEW YORK UNIVERSITY, New York, NY (US)

(72) Inventors: Liam J. Holt, New York, NY (US); Cindy Hernandez, New York, NY (US)

(73) Assignee: NEW YORK UNIVERSITY, New York, NY (US)

(21) Appl. No.: 19/258,211

(22) Filed: Jul. 2, 2025

Related U.S. Application Data
(60) Provisional application No. 63/666,994, filed on Jul. 2, 2024.

(51) Int. Cl. G01N 21/64; B82Y 5/00; C07K 14/32; C12N 15/81; C12N 15/86

(52) U.S. Cl. CPC G01N 21/6486; C07K 14/32; C12N 15/81; C12N 15/86; B82Y 5/00; C07K 2319/43; C07K 2319/60; C12N 2740/15043

(57) ABSTRACT

The invention relates to bright genetically encoded multimeric nanoparticles (GEMs) for physical analysis of the cell interior.

### CROSS-REFERENCE TO RELATED APPLICATIONS

This application claims priority to U.S. Provisional Patent Application No. 63/666,994, filed on Jul. 2, 2024, the disclosure of which is herein incorporated by reference in its entirety.

### STATEMENT REGARDING FEDERALLY SPONSORED RESEARCH

This invention was made with government support under R01 GM132447 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Jun. 26, 2025, is named 243735_000438_SL.xml and is 44,260 bytes in size.

### FIELD OF THE INVENTION

The present invention relates to bright genetically encoded multimeric nanoparticles (GEMs) for physical analysis of the cell interior.

### BACKGROUND

Macromolecular crowding (R. John Ellis, A characteristic of the interiors of all cells is the high total concentration of macromolecules they contain. Trends in Biochemical Sciences, 26:597, 2001), is a phenomenon that describes the significant presence of macromolecules in the intracellular environment, which can constitute up to 40% of the overall cell volume (S. B. Zimmerman and S. O. Trach, Estimation of macromolecule concentrations and excluded volume effects for the cytoplasm of Escherichia coli. J. Mol. Biol., 222:599-620, 1991; J. Spitzer and B. Poolman, How crowded is the prokaryotic cytoplasm? FEBS Lett., 587:2094-2098, 2013). Previous studies have shown that crowding is linked to phase separation processes, such as the concentration of tubulin proteins to aid in microtubule nucleation (J. B. Woodruff et al., The centrosome is a selective condensate that nucleates microtubules by concentrating tubulin. Cell, 169:1066-1077, 2017). However, aberrant phase separation that occurs in highly crowded cellular environments has been associated with various detrimental effects, including genomic instability, altered gene expression, DNA damage, disrupted cell signaling, cellular senescence, and compromised protein quality control (S. Alberti and A. A. Hyman, Biomolecular condensates at the nexus of cellular stress, protein aggregation disease, and ageing. Nat Rev Mol Cell Biol, 22 (3): 196-213, 2021). Thus, understanding the mechanisms behind cellular crowding are crucial for maintaining cellular homeostasis and ensuring the fidelity of essential biological processes.

To explore the effects of molecular crowding in cells, it is crucial to investigate the mesoscale size range, which encompasses macromolecules that contribute to important cellular processes such as diffusion, stabilization of intermolecular complexes, and protein folding (Zhou Huang-Xiang et al., Macromolecular crowding and confinement: biochemical, biophysical, and potential physiological consequences, Ann Rev Biophys. 2008). A current challenge to study cellular crowding is the shortage of tools to study macromolecular complexes of different sizes. Furthermore, approaches such as microinjection of tracer particles into cells inadvertently dilutes the cytoplasm, which makes it difficult to know whether changes in crowding are due to inherent changes in the cell, or changes due to perturbations as a result of the method being used.

The cell interior is packed with macromolecules of mesoscale size, and this crowded milieu significantly influences cellular physiology. Cellular stress responses almost universally lead to inhibition of translation, resulting in polysome collapse and release of mRNA. The released mRNA molecules condense with RNA binding proteins, to form membraneless RNA-protein (RNP) condensates known as processing bodies and stress granules.

The cytoplasm undergoes rapid changes in its physical properties in response to stress. Notably, when exposed to ATP depletion by chemicals or glucose starvation, the cytoplasm of bacterial and yeast cells can undergo a solid-state transition characterized by a significant reduction in macromolecular motion (1-3). However, an immediate and uniform slowdown of all cellular processes might not be helpful in all stress conditions, since there are many processes that must be coordinated in a time-dependent manner to achieve stress adaptation. For example, stress responsive transcription factors translocate from the cytosol to the nucleus within minutes of Saccharomyces cerevisiae being exposed to environmental stresses, activating stress response transcription (4-7). Also, the cell significantly rearranges the plasma membrane (8), vacuole membrane (9) and interorganelle membranes such as nuclear-vacuole junctions (10) in response to glucose starvation. Finally, new structures form within the cytoplasm upon stress. For example, some misfolded proteins are directed to Q-bodies, which are membraneless organelles that form upon heat stress and glucose starvation (11-14). Formation of Q-bodies depends on the interactions between the HSP42 chaperone and misfolded proteins (15). This large-scale remodeling of the cell in response to stress is incompatible with an immediate solidification of the cytoplasm. Therefore, more detailed studies of the physical properties of the cytoplasm during the initial response to stress are necessary.

Cells are highly crowded with mesoscale (10 nm-1 μm diameter) particles (16,17) (FIG. 12A). Crowding both hinders mesoscale motion and drives assembly of mesoscale structures (18,19). Active biological processes also influence the motion of mesoscale particles. For example, the dynamic assembly of microtubules enhances mesoscale diffusivity within the densely packed metaphase spindle (20). This crowded, active environment is crucial for efficient cellular functions, but the mechanisms controlling these biophysical factors and their influence on biochemistry are only beginning to be understood.

Ribosomes, with a diameter of 25 nm, were identified as dominant mesoscale crowders in the cytoplasm, accounting for approximately half of the total excluded volume (around 20% of the total cytoplasmic volume) (21). Inhibition of the TORC1 kinase reduced the concentration of ribosomes, thereby decrowding the cytoplasm, which both increased the diffusivity of mesoscale particles and influenced the formation of phase-separated condensates (21). However, actively translating ribosomes are further organized into polysome complexes, which vary in size depending on the number of ribosomes bound to the mRNA. These higher order cytoplasmic structures might create elastic confinement that could restrict diffusivity of mesoscale particles.

In response to many environmental stresses, there is a general suppression of protein production, primarily by inhibiting the rate-limiting step of translation initiation (22-24). In both yeast and mammalian cells, multiple stresses including glucose starvation, amino acid starvation, and oxidative stress trigger a significant reduction of translation within minutes, leading to disassembly of polysome complexes into monosomes, and the release of mRNA (25-27). The mRNA released from polysomes upon stress is rapidly bound by proteins and forms ribonucleoprotein (RNP) condensates including stress granules (SG) and processing bodies (P-bodies) (28,29). These condensates vary in size from 100 nm to microns in diameter depending on the stress condition and cell type (30,31), some of which are diffraction limited and therefore undetectable by conventional light microscopy (31-34). Polysomes and mRNA are extremely abundant in the cytoplasm, and therefore changes in the assembly state of ribosomes (from polysomes to monosomes) and the redistribution of mRNA to RNP condensates could impact the biophysical properties of the cytoplasm.

### SUMMARY OF THE INVENTION

Various non-limiting aspects and embodiments of the invention are described below.

Provided herein is an isolated polynucleotide encoding a fusion polypeptide comprising an encapsulin domain from Quasibacillus thermotolerans (“QtE”) and a fluorescent tag, wherein the fluorescent tag is located to the C-terminus of the QtE.

In certain embodiments, QtE comprises an amino acid sequence SEQ ID NO: 1 or a functional fragment thereof or a sequence that has at least 90% amino acid sequence identity thereto. In certain embodiments, QtE comprises the amino acid sequence SEQ ID NO: 1. In certain embodiments, QtE consists of the amino acid sequence SEQ ID NO: 1.

In certain embodiments, the fluorescent tag is selected from red fluorescent protein mScarlet, green fluorescent protein (GFP), green fluorescent protein variant Sapphire, self-labeling HaloTag, and functional fragments and derivatives thereof. In certain embodiments, the fluorescent tag is Sapphire which comprises an amino acid sequence SEQ ID NO: 3. In certain embodiments, Sapphire consists of the amino acid sequence SEQ ID NO: 3. In certain embodiments, the fluorescent tag is GFP which comprises an amino acid sequence SEQ ID NO: 5. In certain embodiments, GFP consists of the amino acid sequence SEQ ID NO: 5. In certain embodiments, the fluorescent tag is HaloTag which comprises an amino acid sequence SEQ ID NO: 8. In certain embodiments, HaloTag consists of the amino acid sequence SEQ ID NO: 8. In certain embodiments, the fluorescent tag is mScarlet which comprises an amino acid sequence SEQ ID NO: 10. In certain embodiments, mScarlet consists of the amino acid sequence SEQ ID NO: 10.

In certain embodiments, the fluorescent tag is attached to the C-terminus of the QtE via a linker. In certain embodiments, the linker is a GS-containing linker. In certain embodiments, the linker comprises an amino acid sequence SGSGSGSGSGSG (SEQ ID NO: 2). In certain embodiments, the linker comprises an amino acid sequence GGSGGSGSGGSG (SEQ ID NO: 4).

In certain embodiments, the fusion polypeptide further comprises a purification tag. In certain embodiments, the purification tag is located to the C-terminus of the fluorescent tag. In certain embodiments, the purification tag is a FLAG tag. In certain embodiments, the FLAG tag comprises an amino acid sequence SEQ ID NO: 6. In certain embodiments, the purification tag is attached to the C-terminus of the fluorescent tag via a linker. In certain embodiments, the linker is a GS-containing linker. In certain embodiments, the linker comprises an amino acid sequence SGSGSGSGSGSG (SEQ ID NO: 2). In certain embodiments, the linker comprises an amino acid sequence GGSGGSGSGGSG (SEQ ID NO: 4).

In certain embodiments, the fusion polypeptide further comprises a sequence from the PopZ protein from Caulobacter crescentus, wherein the PopZ sequence is located to the N terminus of QtE and comprises the C-terminal helical oligomerization domain of PopZ or a functional fragment thereof or a sequence that has at least 90% amino acid sequence identity thereto. In certain embodiments, the sequence from the PopZ protein comprises an amino acid sequence SEQ ID NO: 9. In certain embodiments, the sequence from the PopZ protein consists of the amino acid sequence SEQ ID NO: 9.

In certain embodiments, upon its expression in a host cell the fusion polypeptide forms genetically encoded multimeric nanoparticles (GEMs) having a diameter of about 50 nm. In certain embodiments, upon its expression in a host cell the fusion polypeptide forms GEMs having a diameter of 45-55 nm. In certain embodiments, upon its expression in a host cell the fusion polypeptide forms GEMs having a diameter of 47-53 nm. In certain embodiments, upon its expression in a host cell the fusion polypeptide forms GEMs having a diameter of 48-52 nm. In certain embodiments, upon its expression in a host cell the fusion polypeptide forms GEMs having a diameter of 51 nm. In certain embodiments, the host cell is a yeast cell. In certain embodiments, the host cell is a mammalian cell.

In certain embodiments, the fusion polypeptide comprises the amino acid sequence SEQ ID NO: 11. In certain embodiments, the polynucleotide encoding said fusion polypeptide comprises the nucleotide sequence SEQ ID NO: 17.

In certain embodiments, the fusion polypeptide comprises the amino acid sequence SEQ ID NO: 12. In certain embodiments, the polynucleotide encoding said fusion polypeptide comprises the nucleotide sequence SEQ ID NO: 18.

In certain embodiments, the fusion polypeptide comprises the amino acid sequence SEQ ID NO: 13. In certain embodiments, the polynucleotide encoding said fusion polypeptide comprises the nucleotide sequence SEQ ID NO: 19.

In certain embodiments, the fusion polypeptide comprises the amino acid sequence SEQ ID NO: 14. In certain embodiments, the polynucleotide encoding said fusion polypeptide comprises the nucleotide sequence SEQ ID NO: 16.

In certain embodiments, the fusion polypeptide comprises the amino acid sequence SEQ ID NO: 15. In certain embodiments, the polynucleotide encoding said fusion polypeptide comprises the nucleotide sequence SEQ ID NO: 20.

In certain embodiments, the fusion polypeptide comprises the amino acid sequence SEQ ID NO: 23. In certain embodiments, the polynucleotide encoding said fusion polypeptide comprises the nucleotide sequence SEQ ID NO: 21.

In certain embodiments, the fusion polypeptide comprises the amino acid sequence SEQ ID NO: 24. In certain embodiments, the polynucleotide encoding said fusion polypeptide comprises the nucleotide sequence SEQ ID NO: 22.

In certain embodiments, the fusion polypeptide comprises the amino acid sequence SEQ ID NO: 26. In certain embodiments, the polynucleotide encoding said fusion polypeptide comprises the nucleotide sequence SEQ ID NO: 25.

Further provided herein is a fusion polypeptide encoded by any of the above polynucleotides.

Additionally provided herein is a genetically encoded multimeric nanoparticle (GEM) comprising the fusion polypeptide as described above. In certain embodiments, the GEM has a diameter of about 50 nm. In certain embodiments, the GEM has a diameter of 45-55 nm. In certain embodiments, the GEM has a diameter of 47-53 nm. In certain embodiments, the GEM has a diameter of 48-52 nm. In certain embodiments, the GEM has a diameter of 51 nm.

Also provided herein, is a vector comprising any of the above polynucleotides.

In certain embodiments, the vector is a mammalian expression vector. In certain embodiments, the vector is a viral vector. In certain embodiments, the viral vector is a lentiviral vector. In certain embodiments, the polynucleotide encoding the fusion polypeptide is operably linked to a mammalian Ubiquitin C (UBC) promoter.

In certain embodiments, the vector is a yeast expression vector. In certain embodiments, the yeast expression vector is a yeast integration expression vector. In certain embodiments, the polynucleotide encoding the fusion polypeptide is operably linked to a yeast transcription factor (INO4) promoter or a yeast histidine synthesis (His3) promoter.

Further provided herein is a host cell comprising any of the above polynucleotides or any of the above vectors.

Additionally provided herein is a method of producing genetically encoded multimeric nanoparticles (GEMs) comprising:
a) incubating the host cell as described above under conditions when the fusion polypeptide is produced and assembles into GEMs, and
b) isolating the GEMs.

Further provided herein is a method for probing a biophysical property of a cell, the method comprising transforming, transfecting, or transducing the cell with any of the above polynucleotides or any of the above vectors and detecting the fluorescent tag.

In some embodiments, the biophysical property is selected from cytoplasmic fluidity, effective diffusivity, anomalous diffusion exponent, viscoelastic/mechanical state, cytoplasmic crowding, spatial heterogeneity, confinement/caging, non-equilibrium/active fluctuation, phase separation and condensates, and any combination thereof.

In some embodiments, a change in the biophysical property results in a biophysical consequence.

In some embodiments, the biophysical consequence is selected from ATP depletion or metabolic collapse, pH changes, glucose starvation, a change in ribosome concentration and/or polysome disassembly, formation of RNA condensates (P-bodies, stress granules, RNP granules), free RNA abundance, assembly of Q-bodies, and any combination thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

FIGS. 1A-1C show rheological probes and their relative size on the mesoscale and GEM brightness. FIG. 1A shows sizes of various biomolecules in relation to GEMs. FIG. 1B shows histograms of GEMs brightness for yeast. The median brightness for 50 nm-GEMs is significantly higher than that of 40 nm-GEMs. FIG. 1C shows histograms of GEMs brightness for mammalian cells.

FIGS. 2A-2B show Cryo-EM single particle analysis of 50 nm-GEMs. FIG. 2A shows high resolution map of the GEM encapsulin cage (left) and low-pass filtered GEM particle with GFP density (green, right) reveal a GEM with a diameter of 50 nm (scale bar right). FIG. 2B shows 2D projection of Quasibacillus thermotolerans two-component encapsulin-based iron storage compartment (EMDB: 9383, top) and the 50 nm-GEM (as described herein, bottom) shows a clear density outside of the encapsulin cage ascribable to GFP.

FIGS. 3A-3J demonstrate that 50 nm GEMs showed lower effective diffusion compared to 40 nm GEMs in S. Cerevisiae. FIGS. 3A-3B show rainbow tracks of 40 nm-GEMs (PfV) and 50 nm-GEMs (QtE) overlaid with brightfield images of yeast. A jet color scale was used to indicate the Effective Diffusion Coefficient. Blue represents the lowest diffusion coefficient and red the highest diffusion coefficient, demonstrating that 50 nm-GEMs move slower than 40 nm-GEMs. FIGS. 3C-3D show the Step Size Distributions for the two types of GEMs. The y axis is in log scale. Lower t_lags are plotted darker. Gray area on the left represents the pixel size as a measure of uncertainty. The inset shows the α2 parameter for the different t_lags. The sudden drop on t_lag 1 was probably an artifact due to the linking of the trajectories when tracking. Distributions appeared to be Gaussian. FIG. 3E shows the medians of the step size distribution for different t_lags. 50 nm-GEMs have lower medians than 40 nm-GEMs. The p-value comes from the Mann-Whitney test on the median and is reported on the right y axis. All the differences in the median are statistically significant. FIG. 3F shows mean squared displacement vs tau plot for the range between 10 and 100 ms. 40 nm-GEMs show a higher slope. FIG. 3G shows the Effective Diffusion Coefficient. Each dot represents individual cells. The stars represent the p-value for the Mann-Whitney test on the medians. Values for median diffusivity are displayed under each boxplot. 50 nm-GEMs show lower Diffusion Coefficients than 40 nm-GEMs. FIGS. 3H-3I display α-logD plots that show the anomalous exponent (α) and the logarithm of the diffusion coefficient (logD). The blue dots represent individual tracks, and black dots represent the median track values per cell. The color map represents the Kernel Density Estimation (KDE) which accounts for the spatial density of the data points. Darker shades in the KDE represent a higher concentration of data points around that value. For 50 nm-GEMs, a high trajectory spread was observed due to cell heterogencity. In contrast, 40 nm-GEMs exhibited less heterogencity and a narrower data spread. Moreover, the dashed line at α=1 indicates the trajectory reference point for Brownian motion. 40 nm-GEMs trajectories were centered around α=1, indicating Brownian motion of these particles. In contrast, 50 nm-GEM trajectories appeared as two subpopulations, with one population centering around α=1, and the other centering around lower a values, indicating subdiffusive motion. FIG. 3J shows the comparison of the brightness of the 40 nm-GEMs and 50 nm-GEMs. The 50 nm-GEMs are significantly brighter and this makes them more widely useful with less specialized microscopes.

FIGS. 4A-4I reveal 50 nm GEMs show lower effective diffusion compared to 40 nm GEMs in Hela cells. FIGS. 4A-4B show rainbow tracks of 40 nm-GEMs (PfV) and 50 nm-GEMs (QtE) overlaid with brightfield images of yeast. A jet color scale was used to indicate the Effective Diffusion Coefficient. Blue represents the lowest diffusion coefficient and red the highest diffusion coefficient, demonstrating that 50 nm-GEMs move slower than 40 nm-GEMs. FIGS. 4C-4D show Step Size Distributions for the two types of GEMs. The y axis is in log scale. Higher Taus are represented for both GEM types. FIG. 4E shows the medians of the step size distribution for different t_lags. 50 nm-GEMs have lower medians than 40 nm-GEMs. The p-value comes from the Mann-Whitney test on the median and is reported on the right y axis. All the differences in the median are statistically significant. FIG. 4F shows the mean squared displacement vs tau plot for the range between 10 and 100 ms. 40 nm-GEMs show a higher slope. FIG. 4G shows Effective Diffusion Coefficient. Each dot represents individual cells. The stars represent the p-value for the Mann-Whitney test on the medians. Values for median diffusivity are displayed under each boxplot. 50 nm-GEMs show lower Diffusion Coefficients than 40 nm-GEMs. FIGS. 4H-4I show α-logD plots depicting the anomalous exponent (α) and the logarithm of the diffusion coefficient (logD). The blue dots represent individual tracks, and black dots represent the median track values per cell. The color map represents the Kernel Density Estimation (KDE) which accounts for the spatial density of the data points. Darker shades in the KDE represent a higher concentration of data points around that value. The plot shows a bidimensional parameter space of the parameters of the anomalous equation MSD=2Dtau\hat{ }α. There is a high trajectory-trajectory heterogeneity in both: α and logD. Cell-cell heterogeneity is much lower as seen by the spread of the black circles. The dashed lines represent the median from the group of cells in both axes.

FIGS. 5A-5K. FIG. 5A shows the cloning of the QtE encapsulin (purple) and GFP (green) sequence in a yeast expression vector. FIG. 5B shows bright field images of the yeast S. cerevisiae BY4741. FIG. 5C shows cell expressing GEMs. Each green dot corresponds to a GEM particle. FIG. 5D shows GEM tracks projection. FIGS. 5E-5F show mean squared displacement as a function of tau (time between frames). The function was compared to Brownian diffusion (dashed line). Between 0.01 s and 0.1 s most of the tracks were present. After 0.1 s there was too much noise due to the low number of tracks. 40 nm-GEM and 50 nm-GEM showed similar behavior. FIGS. 5G-5H show Velocity Autocorrelation Function (Cv) as a function of the time between displacements. Constant negative peaks show subdiffusive signature. 40 nm-GEM and 50 nm-GEM show similar behavior. FIGS. 51-5J show angle (degrees) distribution at different t lags. The distribution was almost uniform, showing the slight appearance of a peak on 180 degrees. Inset shows Kolmogorov-Smirnov test between consecutive distributions. Consistent distributions at different t lags were observed. Kolmogorov Smirnov test shows distributions are not different. FIG. 5K shows-log 10 of the p-value of the Kolmogorov-Smirnov test. Red dashed line shows the limit above which distributions can be assumed as different. Since all dots are below the red dashed lines, distributions of 40 nm-GEM and 50 nm-GEM cannot be assumed as different.

FIG. 6 shows the yeast expression plasmid designed for expression of 50 nm GEM nanoparticles.

FIG. 7 shows the yeast expression plasmid designed for protein purification of 50 nm GEM nanoparticles via FLAG peptide.

FIG. 8 shows the plasmid designed for lentiviral packaging and mammalian expression of 40 nm GEM nanoparticles.

FIG. 9. shows the plasmid designed for lentiviral packaging and mammalian expression of 50 nm GEM nanoparticles.

FIG. 10 shows 40 nm GEM (“PfV”) structure (right) and 50 nm GEM (“QtE”) structure (left).

FIGS. 11A-11K show mesoscale μNS particles and 40 nm-GEMs dynamics in wild type S. cerevisiae cells. FIG. 11A shows step size distribution of μNS particles at different time intervals. FIG. 11B shows step size distribution of μNS particles at τ=1 s and 40 nm-GEMs at τ=100 ms (μNS particles: n=10,437 trajectories; 40 nm-GEMs: n=6,382 trajectories). FIG. 11C shows ensemble-averaged mean-squared displacement (MSD) versus time delay (τ), log₁₀ scale. A linear model was fitted to determine the anomalous exponent α values for 40 nm-GEMs in the indicated conditions at 30 minutes (glucose: n=6,382 trajectories; no glucose: n=5,116 trajectories; no amino acids: n=716 trajectories; H₂O₂ stress: n=986 trajectories). FIGS. 11D-11G show normalized velocity autocorrelation with respect to τ=0 sec for μNS particles, with correlations calculated at different time intervals (0-1 sec, blue to red) for wild type yeast cells in glucose rich medium, after 30 minutes of glucose starvation, acute amino acids starvation and H₂O₂ stress in S. cerevisiae cells. The inserted graph displayed the velocity autocorrelation value at different time scales with the indicated conditions. FIGS. 11H-11K show normalized velocity autocorrelation with respect to τ=0 s for 40 nm-GEMs at different time intervals (0.01-0.1 sec, blue to red) for wild type yeast cells in glucose rich medium, after 30 minutes of glucose starvation, acute amino acids starvation and H₂O₂ stress in S. cerevisiae cells. The inserted graph displayed the velocity autocorrelation value at different time scales with the indicated conditions.

FIGS. 12A-12J show diverse stresses lead to a transient increase in diffusivity of mesoscale particles. FIG. 12A shows mesoscale rheological probe sizes in relation to biological length-scales. FIGS. 12B-12D show the effective diffusivity at one second (D_{eff-1s}) for μNS particles after acute glucose starvation (left), oxidative H₂O₂ stress (middle, synthetic complete medium with 2% glucose and 0.3 mM H₂O₂ as oxidative stress) and acute amino acid starvation (right, media with 2% glucose and no amino acids) in S. cerevisiae cells (Each datapoint represents one cell, N=3 biological replicates, n>100 cells. Boxes represent the 5-95% confidence interval, median value indicated below each box. FIG. 12E shows the ensemble-averaged mean-squared displacement (MSD) versus time delay (τ), log₁₀ scale. A linear model was fit to determine the anomalous exponent α values for μNS particles in the indicated conditions at 30 minutes (glucose: n=10,437 trajectories; no glucose: n=16,372 trajectories; no amino acids: n=1589 trajectories; H₂O₂ stress: n=558 trajectories). FIG. 12F shows normalized velocity autocorrelation with respect to τ=0 s for μNS particles, with correlations calculated over different tracking time intervals (0-1 sec, blue to red) for wild type yeast cells in glucose rich medium. FIG. 12G shows the summary of velocity autocorrelation values at t=0.1 s for various conditions at 30 min in wild type yeast cells. FIGS. 12H-12J show effective diffusivity at 100 msec (D_{eff-100ms}) for 40 nm-GEMs upon acute glucose starvation, acute amino acids starvation and H₂O₂ stress in S. cerevisiae cells. (Each datapoint represents one cell. N=3 biological replicates, n>100 cells, boxes represent the 5-95% confidence interval, median value below each box.) All P values determined by one-way ANOVA, **:p<0.01; ***:p<0.001; ****:p<0.0001)

FIGS. 13A-13D show cell and cytoplasmic volumes do not change upon 30 minute glucose starvation in osmotically balanced media. FIG. 13A shows representative images of live wild type S. cerevisiae cells with fluorescent protein markers labeling the plasma membrane and vacuole membrane. FIGS. 13B-13D show quantification of (FIG. 13B) total cell volume (V_{c}), (FIG. 13C) vacuole volume (Vᵥ), and (FIG. 13D) cytosolic volume (V_{c}−Vᵥ) (control: n=147 cells; no glucose: n=140 cells, statistical comparison was performed using nonparametric Mann-Whitney test, ns=not significant).

FIGS. 14A-14G show ATP reduction and cytoplasmic acidification decrease mesoscale particle diffusivity. FIG. 14A shows a graphic illustration of QUEEN sensor. The QUEEN ATP sensor displays a bimodal excitation spectrum with peaks at 410 nm (ATP-bound) and 480 nm (ATP-unbound) and an emission maximum at 510 nm. Representative QUEEN ratio (410 nm cx/480 nm ex) images of yeast cells in glucose rich and glucose depleted conditions are shown. The QUEEN ratio is pseudo-colored. FIG. 14B shows time course analysis of the QUEEN ratio after glucose depletion (n>1,000 cells for each time point, mean±SEM). FIG. 14C shows pH calibration curve determined using permeabilized S. cerevisiae cells expressing cytosolic pHluorin sensor. A linear model was used to relate pHluorin fluorescence ratios to intracellular pH (n>500 cells for each pH condition). FIG. 14D shows dynamics of intracellular pH in S. cerevisiae cells after acute glucose starvation (n>590 cells for each time point, mean±SEM). FIG. 14E shows time and ensemble-averaged mean-squared displacement (MSD) versus time delay (τ), log 10 scale. The anomalous exponent α values was determined from the power-law fit for glucose rich (glucose) or ATP depleted (ATP depletion) for μNS particles and 40 nm-GEMs at 30 minutes (ATP depleted condition: synthetic complete medium buffer at pH 5.5 without glucose, 20 mM 2-deoxyglucose and 10 μM antimycin A and 80 mM sorbitol were supplemented. Trajectories analyzed for μNS particles, glucose: n=16373 trajectories; ATP depletion: n=16373 trajectories; For 40 nm-GEMs, glucose: n=2620 trajectories; ATP depletion: n=3176 trajectories). FIG. 14F shows step size distribution of UNS particles in one second time scale, 40 nm-GEMs in 100 msec time scale in glucose rich and ATP depleted conditions at 30 minutes (For μNS particles, glucose: n=16373 trajectories; ATP depletion: n=16373 trajectories; For 40 nm-GEMs, glucose: n=2620 trajectories; ATP depletion: n=3176 trajectories). FIG. 14G shows the fold change of median effective diffusivity at τ=1 sec for μNS particles, and 100 msec (D_{eff-100ms}) for 40 nm-GEMs from 3 biological replicate experiments (mean±SEM.) upon ATP depletion conditions in S. cerevisiae cells.

FIGS. 15A-15J show polysome disassembly is necessary for increased mesoscale diffusivity in response to stress. FIG. 15A shows polysome profile analysis by fractionation of cell extracts on a sucrose gradient. Cells were collected 30 minutes after switching to the indicated conditions (CHX: 100 μg/ml cycloheximide just prior to glucose starvation). FIG. 15B shows quantification of polysome profiles: the area under the curve was used to determine the relative amounts of 80S monosomes and polysomes, N=3 biological replicates. FIG. 15C shows a schematic of polysome rearrangement upon acute glucose starvation with or without cycloheximide. Green: translation initiation complexes (TIC), cyan: ribosomes, blue: RNA, orange: cycloheximide. FIG. 15D shows effective diffusivity at τ=1 s (D_{eff-1s}) for μNS particles after pretreatment with cycloheximide followed by 30 minutes in normal glucose, acute glucose starvation, acute amino acid starvation, or H₂O₂ stress in S. cerevisiae cells (each datapoint represents one cell, n>100 cells; N>3 biological replicates for each condition; boxes represents the 5-95% confidence interval, median value indicated above each box, P values determined by one-way ANOVA, ns: not significant; ****:p<0.0001). FIG. 15E shows the normalized velocity autocorrelation at tracking τ=0.1 s after 30 min in the indicated conditions in wild type yeast cells, with or without 100 μg/ml cycloheximide. FIG. 15F shows a representative filtered 2D slice of a cryo-electron tomogram from S. cerevisiae in control glucose medium. Top: Cytosol is crowded with dense globular ribosomes (single ribosome encircled in white). Bottom: Monosomes (light blue) and polysomes (dark blue) annotated based on refined positions and orientations derived from subtomogram averaging in 3D. ER=endoplasmic reticulum. FIG. 15G shows polysome counts at 30 minute acute glucose starvation as a fraction of the polysome counts in the control condition for sucrose gradient fractionation and cryo-ET data. FIG. 15H shows representative 3D annotations of a short (disome) and long (6-some) polysome. FIG. 15I shows representative image of coarse-grained molecular dynamics (MD) framework. In this example, ribosomes occupy 30% of total box volume, 75% of which are modeled as polysomes (dark blue) and 25% as monosomes (gray), 125 nm particles are shown in green. FIG. 15J shows the most probable root mean squared (rms) displacement after t=100 simulation time units for particles of various sizes simulated with 0, 25%, 75% and 100% ribosomes as polysomes. Error bars are SD; N=5.

FIGS. 16A-16J show that Cryo-ET of S. cerevisiae confirmed polysome collapse in early stages of acute glucose starvation. FIG. 16A shows scanning electron microscopy (SEM) view of S. cerevisiae cells plunge-frozen on an electron microscopy grid. The view showed a single grid square. FIG. 16B shows focused ion beam (FIB) view of the same grid square showing the homogenous distribution of yeast cells. Milling patterns (dashed boxes) indicate the areas targeted for FIB ablation. FIGS. 16C-16D show FIB view of the cells thinned down to approximately 1 μm (FIG. 16C, arrowheads) and an SEM view of the final lamella with thickness of around 200 nm (FIG. 16D). Micro-expansion joints positioned on either side of the lamella reduced tension and lamella bending. FIG. 16E shows Cryo-TEM overview of the lamella shown in FIG. 16D. 6 positions were used (dashed boxes) for cryo-ET acquisition. FIG. 16F shows the distance from the mRNA exit site of one ribosome (red) to the mRNA entry site of the nearest neighbor (blue) that was measured using the refined poses (position and orientations) derived from subtomogram averaging. FIG. 16G shows distance distribution of mRNA exit site of one ribosome to the mRNA entry site of the nearest neighbor across all ribosomes localized in the presence of glucose (left) and acute glucose starvation for 30 minutes (right). Fitting a gaussian distribution to the data for distances between 0 to 8 nm best described the polysome peak in the control data. Thus, a distance cutoff of 8 nm between neighboring mRNA exit and entry sites was chosen to assign ribosomes either to the monosome or polysome fraction in the glucose control and acute glucose starvation condition. FIG. 16H shows polysome fractions detected in the glucose rich and depletion conditions as a function of distance thresholds. FIG. 16I shows subtomogram averages of cytosolic ribosomes from glucose rich condition (estimated resolution of 17.8 Å based on 2× binned subtomograms with a pixel size of 6.85 Å/px) and glucose starvation (estimated resolution of 10.2 Å, unbinned data at 3.425 Å/px) depicted at low intensity threshold. The average in glucose-rich conditions shows densities of neighboring ribosomes (arrowheads) characteristic for polysome engagement that are absent in the acute glucose starvation cells. FIG. 16J shows distribution of polysome lengths for glucose control cells and acute glucose starvation cells. Note that in the control condition, a single polysome consisting of 10 ribosomes was detected, which is not included in the plot.

FIGS. 17A-17H show that formation of stress granules and P-bodies is required for mesoscale fluidization of the cytoplasm. FIG. 17A shows schematic of P-body and stress granule formation. Green: translation initiation complexes; cyan: ribosomes; blue: RNA; pink: RNA binding proteins. FIG. 17B shows representative images of cells with P-body (Dcp2-mScarlet) and stress granule (Pab1-mNeoGreen) markers during acute glucose starvation. Cycloheximide (CHX) pre-treatment inhibits the formation of both RNP condensates. FIGS. 17C-17E show the effective diffusivity at one second (D_{eff-1s}) for μNS particles in S. cerevisiae cells (Each datapoint represents one cell, n>100 cells, N=3 replicates for each condition; boxes represent the 5-95% confidence interval; median value indicated below each box, P values were determined by one-way ANOVA, ns: not significant; *:p<0.05; ***:p<0.001; ****:p<0.0001). FIGS. 17F-17G show values at τ=0.1 s for normalized velocity autocorrelation that were extracted for the indicated conditions upon 30 min treatment in wild type, edc3Δ and pab1ΔP yeast cells. FIG. 17H shows normalized velocity autocorrelations at the first simulation time unit for all indicated simulation conditions. “Normal growth conditions” were simulated as 75% polysome to monosome ratio without free RNA. “Glucose starvation conditions” were simulated as 25% polysome to monosome ratio with varying concentrations of free RNA (from 0-5 μM).

FIGS. 18A-18I show stress granules and P-bodies dynamics upon diverse types of stresses in S. cerevisiae cells, and molecular dynamic simulation of the effect of RNA. FIG. 18A shows representative fluorescent live cell images showing P-body (Dcp2-mScarlet) and stress granule (Pab1-mNcoGreen) markers after 30 minute amino acid starvation or oxidative H₂O₂ stress. FIGS. 18B-18D show time course showing dynamics of P-body formation (Dcp2-mScarlet marker) after the indicated stresses in wild type and edc3Δ S. cerevisiae cells. FIGS. 18E-18F show that P-bodies (Dcp2-mScarlet marker) are not colocalized with μNS particles or 40 nm-GEMs (BF=Bright field). FIGS. 18G-18I show normalized velocity autocorrelation with respect to τ=0 s for 125 nm particles at different simulation time units (0-10, blue to red) in the indicated simulation framework.

FIGS. 19A-19E show increased mesoscale diffusivity is crucial for Q-body assembly upon acute glucose starvation. FIG. 19A shows timeline for experiments to correlate early μNS particle tracking (at 10-15 min) with Q-body assembly measurement (at 70-75 min) in the same yeast cell. FIG. 19B shows correlation between effective diffusivity for μNS particles (D_{eff-1sec}) 15 min after acute glucose starvation with the total intensity of Hsp42 foci in the same cell 75 min after acute glucose starvation (n=128 cells). FIG. 19C shows representative images of the dynamics of Q-body formation as indicated by Hsp42-mScarlet in wild type and edc3Δ S. cerevisiae cells after acute glucose starvation. Hypotonic shock was achieved by overnight culture of yeast cells in synthetic complete media with 2% glucose and 500 mM KCl to log phase (O.D. 600 nm<0.4) and then switching to glucose starvation media with no KCl. FIG. 19D shows average number of Hsp42 foci per cell after acute glucose starvation in the indicated conditions (wild type-glucose: n=122 cells; edc3Δ-glucose: n=101 cells; wild type-glucose, hypotonic shock: n=103 cells; edc3Δ-glucose, hypotonic shock: n=86 cells, N=3 biological replicates on independent days, mean±SEM.). FIG. 19E shows graphic illustration of Q-body assembly. Cyan: ribosomes; blue: RNA; pink: P-body; green: Q-bodies.

FIGS. 20A-20L show mesoscale μNS particle diffusivity increases upon hypotonic shock. FIGS. 20A-20B show the distribution of average particle intensities for μNS particles in the indicated conditions (n>700 trajectories in each condition) for wildtype and edc3Δ cells. FIG. 20C shows effective diffusivity at one second (D_{eff-1s}) for μNS particles from 3 biological replicate experiments in the indicated conditions in S. cerevisiae cells (each datapoint represents one cell, n>100 cells were collected for each condition, the boxes represent the 5-95% confidence interval, the median value was indicated above each distribution). FIGS. 20D-20E show polysome profile analysis by fractionation of cell extracts on a sucrose gradient in the indicated conditions. Cells were collected at 30 minutes upon switching to the respective medium (control: synthetic complete medium with 2% glucose, no glucose: synthetic complete medium with 2% sorbitol). FIG. 20F shows quantification of the area under the curve of 80S monosomes and polysomes to calculate the ratio between the two fractions from three biological replicates. FIGS. 20G-20J show time course analysis of the QUEEN ratiometric ATP sensor in the indicated conditions (n>500 cells for each condition). FIG. 20K shows time course showing formation of P-bodies (Dcp2-mScarlet marker) after acute glucose starvation together with hypotonic shock in wild type and edc3Δ yeast cells. FIG. 20L shows quantification of the number of Dcp2-mScarlet foci per cell at the indicated time points (wild type-glucose: n=64 cells; edc3Δ-glucose: n=51 cells; wild type-glucose, hypotonic shock: n=200 cells; edc3Δ-glucose, hypotonic shock: n=200 cells, from 3 biological replicates, Mean±SEM).

FIGS. 21A-21I show partitioning of mRNAs into stress granules increases mesoscale particle diffusivity in mammalian cells. FIGS. 21A-21B show effective diffusivity at 100 msec (D_{eff-100ms}) for 40 nm-GEMs upon sodium arsenite and heat stress. N=3 biological replicates; n>50 cells for all conditions. Boxes represent 5-95% confidence intervals. FIG. 21C shows schematic of predicted changes to polysomes and mRNA upon stress, with and without cycloheximide. Green: translation initiation complex; Cyan: ribosomes; blue: mRNA; pink: stress granule; magenta: cycloheximide. FIG. 21D shows representative poly-dT FISH images showing spatial distribution of mRNA in control and G3BP 1/2 dKO (double knockout) U2OS cells after 10 nM DMDA-PatA treatment for 30 minutes. Hoechst dye was used to stain DNA. Yellow arrows indicate mRNA foci. FIGS. 21E-21F show change in effective diffusivities (D_{eff-100ms}) of 40 nm-GEMs. The median D_{eff-100ms} was measured for the same cell before and 30 minutes after each treatment. Each dot represents the fold change of median diffusivity of one cell; the D_{eff-100ms} in the treatment condition (control=DMSO treatment) was normalized to the D_{eff-100ms} for the same cell prior to treatment (0 minutes). N=3 biological replicates; n>29 cells for all conditions. Boxes represent 5-95% confidence intervals. FIG. 21G shows schematic of the effects of stresses on translation, and the mode of action of ISRIB. FIGS. 21H-21I show effective diffusivity at 100 msec (D_{eff-100ms}) for 40 nm-GEMs upon 200 nM ISRIB, 250 μM sodium arsenite (NaAsO₂) and 10 nM DMDA PatA treatment for 30 min treatment. N=3 biological replicates; n>50 cells for all conditions. Boxes represent 5-95% confidence intervals. Statistical comparisons by one-way ANOVA test, ns=not significant, *p<0.05, **p<0.01, ***p<0.001, ****p<0.0001.

FIGS. 22A-22G show characterization of mesoscale diffusivity upon depletion of mRNA after poly (I:C) treatment, stress granule formation in response to stresses and translation inhibitors. FIG. 22A shows representative immunofluorescence images of stress granules (G3BP1) in U2OS cells treated with indicated protein translation inhibitors for 30 minutes. Hoechst dye was used to stain DNA. FIG. 22B shows representative poly-dT FISH images for poly (A) RNAs in wild type and G3BP1/2 dKO U2OS cells without drug treatment. FIG. 22C shows quantification of poly-dT FISH foci number in the cytoplasm with the indicated conditions (WT control: n=57 cells; WT DMDA-PatA n=45 cells; G3BP1/2 dKO control n=76 cells; G3BP1/2 dKO DMDA-PatA n=67 cells, mean±SD. Statistical comparison was by a one-way ANOVA test. Ns=not significant, *p<0.05, **p<0.01, ****p<0.0001). FIG. 22D shows data from FIGS. 21E-21F with the same cells before and after the indicated treatment connected by lines. Most WT cells have increased diffusivity upon translational inhibition with puromycin. This effect was suppressed if polysome collapse was prevented with cycloheximide, or if stress granule formation was prevented by deletion of G3BP1 and G3BP2 proteins. FIG. 22E shows representative fluorescence images of 50 nm-GEMs stably integrated into G3BP1-GFP, G3BP1/2 dKO U2OS cell line, G3BP1-GFP foci formation in response to Poly (I:C) treatment. FIG. 22F shows ensemble-averaged mean-squared displacement (MSD) versus time delay (τ), log₁₀ scale. A linear model was fitted to determine anomalous exponent α values. FIG. 22G shows representative live cell images of U2OS cells expressing Opto-Control or Opto-PUM, with or without 100 μg/ml cycloheximide treatment while activating with 488 nm blue light laser to drive Opto-PUM condensation.

FIGS. 23A-23G show degradation of RNA or artificially condense RNA increases mesoscale diffusivity in the cytoplasm. FIG. 23A shows mode of action of poly (I:C) induces cytosolic mRNAs degradation. FIG. 23B shows representative poly-dT fluorescence in situ hybridization images of G3BP1-GFP, G3BP1/2 dKO U2OS cells, 2-hour after transfection with 1 μg of Poly (I:C) with 2 μl FuGENE® 6 transfection reagent, control cells were transfected with FuGENE® 6 transfection reagent only. Nuclei were stained by Hoechst, blue in the merged image. FIG. 23C shows quantification of average poly-dT intensity in the cytosol (control: n=73 cells; Poly (I:C) treatment: n=63 cells, statistical comparison was performed using a nonparametric Mann-Whitney test). FIG. 23D shows effective diffusivity at 100 msec (D_{eff-100ms}) for 50 nm-GEMs (control: n=103 cells; Poly (I:C) treatment: n=46 cells, statistical comparison was performed using nonparametric Mann-Whitney test). FIG. 23E shows graphic illustration of blue light inducible opto-control and opto-PUM constructs. FIG. 23F shows representative live cell images of U2OS cells expressing opto-Control or opto-PUM together with 40 nm-GEMs, 488 nm blue light laser pulsed 100 ms at 25% laser power every 5 seconds for a period of 5 minutes to drive opto-PUM condensation. FIG. 23G shows the fold change of median effective diffusivity (D_{eff-100ms}) for 40 nm-GEMs in the indicated conditions. D_{eff-100ms} after 30 min light exposure was normalized to the value in the same cell pre-treatment. N=3 biological replicates; n=26 cells for opto-control, n=43 for opto-PUM, mean±SEM. Statistical comparison was performed using a paired two-tailed t test. *p<0.05.).

FIGS. 24A-24Z show amino acid or nucleotide sequences of the present disclosure. FIG. 24A shows SEQ ID NO: 1 Quasibacillus thermotolerans (Qt)-Encapsulin. FIG. 24B shows SEQ ID NO: 2 6×SG-linker. FIG. 24C shows SEQ ID NO: 3 Sapphire. FIG. 24D shows SEQ ID NO: 4 2×GGS-2×GSG-linker. FIG. 24E shows SEQ ID NO: 5 GFP. FIG. 24F shows SEQ ID NO: 6 FLAG. FIG. 24G shows SEQ ID NO: 7 Pyrococcus furiosis (PfV) Encapsulin mammalian codon optimized. FIG. 24H shows SEQ ID NO: 8 HaloTag. FIG. 24I shows SEQ ID NO: 9 PopZ (popZ3H). FIG. 24J shows SEQ ID NO: 10 mScarlet. FIG. 24K shows SEQ ID NO: 11 Fusion protein including PopZ (popZ3H) (SEQ ID NO: 9), Qt-Encapsulin (SEQ ID NO: 1), Sapphire (SEQ ID NO: 3), and a linker (SEQ ID NO: 2) [encoded by pLH2220]. FIG. 24L shows SEQ ID NO: 12 Fusion protein including PopZ (popZ3H) (SEQ ID NO: 9), Qt-Encapsulin (SEQ ID NO: 1), HaloTag (SEQ ID NO: 8), and a linker (SEQ ID NO: 2) [encoded by pLH2494]. FIG. 24M shows SEQ ID NO: 13 Fusion protein including PopZ (popZ3H) (SEQ ID NO: 9), Qt-Encapsulin (SEQ ID NO: 1), mScarlet (SEQ ID NO: 10), and a linker (SEQ ID NO: 2) [encoded by pLH2328]. FIG. 24N shows SEQ ID NO: 14 Fusion protein including Qt-Encapsulin (SEQ ID NO: 1), Sapphire (SEQ ID NO: 3), and a linker (SEQ ID NO: 2) [encoded by pLH1976]. FIG. 24O shows SEQ ID NO: 15 Fusion protein including Qt-Encapsulin (SEQ ID NO: 1), GFP (SEQ ID NO: 5), FLAG (SEQ ID NO: 6), and a linker (SEQ ID NO: 4) [encoded by pLH2508]. FIG. 24P shows SEQ ID NO: 16 nucleotide coding sequence for SEQ ID NO: 14 (codon-optimized for expression in yeast). FIG. 24Q shows SEQ ID NO: 17 nucleotide coding sequence for SEQ ID NO: 11 (codon-optimized for expression in mammalian cells). FIG. 24R shows SEQ ID NO: 18 nucleotide coding sequence for SEQ ID NO: 12 (codon-optimized for expression in mammalian cells). FIG. 24S shows SEQ ID NO: 19 nucleotide coding sequence for SEQ ID NO: 13 (codon-optimized for expression in mammalian cells). FIG. 24T shows SEQ ID NO: 20 nucleotide coding sequence for SEQ ID NO: 15 (codon-optimized for expression in yeast). FIG. 24U shows SEQ ID NO: 21 PfV-Sapphire nucleotide coding sequence for SEQ ID NO: 23 (codon-optimized for expression in yeast). FIG. 24V shows SEQ ID NO: 22 PIV-Sapphire-FLAG nucleotide coding sequence for SEQ ID NO: 24 (codon-optimized for expression in yeast). FIG. 24W shows SEQ ID NO: 23 PfV-Sapphire polypeptide sequence yeast codon optimized [encoded by pLH497]. FIG. 24X shows SEQ ID NO: 24 PIV-Sapphire-FLAG polypeptide sequence yeast codon optimized [encoded by pLH2608]. FIG. 24Y shows SEQ ID NO: 25 PIV-Sapphire nucleotide coding sequence for SEQ ID NO: 26 (codon-optimized for expression in yeast). FIG. 24Z shows SEQ ID NO: 26 PfV-Sapphire polypeptide sequence yeast codon optimized [encoded by pLH1984].

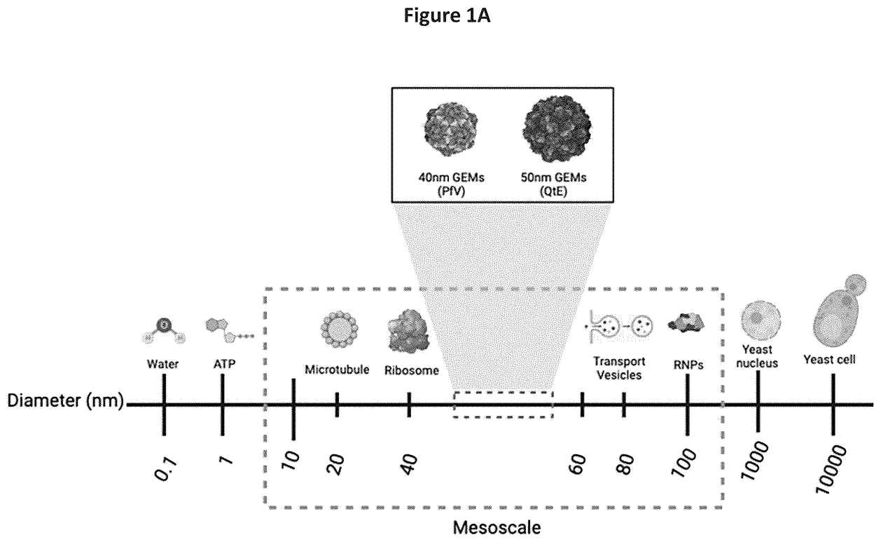

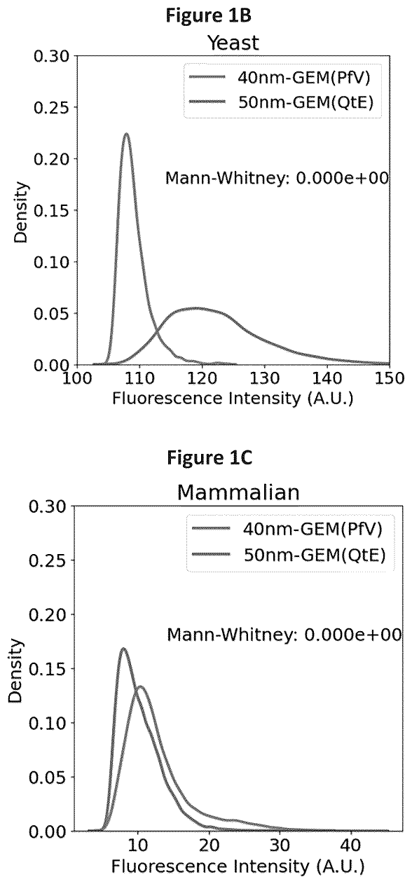

### DETAILED DESCRIPTION

Changes in the biophysical properties of cells have been shown to have effects on the progression of various diseases such as neurodegeneration, cancer, and fibrosis. For example, decreases in protein homeostasis have been linked to progression of Amyotrophic Lateral Sclerosis (ALS). Also, several hallmarks of aging have been related to the formation of aberrant condensates during phase separation (S. Alberti and A. A. Hyman, Biomolecular condensates at the nexus of cellular stress, protein aggregation disease, and ageing. Nat Rev Mol Cell Biol, 22 (3): 196-213, 2021). For these reasons, it is crucial to understand the mechanisms that control the physical properties of the cell and the physiological consequences of perturbations to this environment. Many regulatory macromolecules, such as ribosomes, ribonucleic proteins, and microtubules span the mesoscale size range (10 nm-100 nm). To study the cellular environment at the mesoscale level, genetically encoded multimeric nanoparticles (GEMs) have been designed by fusing scaffold encapsulin proteins from thermostable organisms with fluorescent tags. After translation of the fusion protein, the individual monomers of these nanoparticles self-assemble into stable particles which can be visualized by fluorescence microscopy, and analyzed by single particle tracking. GEM nanoparticles report on the intracellular environment by the use of fluorescent single-particle tracking (M. Delarue et al., mTORC1 controls phase separation and the biophysical properties of the cytoplasm by tuning crowding. Cell, 174 (2): 338-349.e20, 2018; Tobias W Giessen et al., Large protein organelles form a new iron sequestration system with high storage capacity. eLife, 8: e46070, 2019). An important advantage of GEM nanoparticles of the present invention is that they are derived from scaffold proteins from thermostable organisms of a different kingdom than the species that they are being used in. This reduces the possibility of protein-specific interactions between the GEM nanoparticles and native proteins in the cell. By providing GEMs such as 50-nm GEMs derived from bacterium Quasibacillus thermotolerans, the present invention expands the microrheology probe toolkit and expands the available probes within the mesoscale range allowing a deeper insight into the effects of intracellular crowding on macromolecules of larger sizes. Moreover, because the cell is crowded with molecules of varying sizes, different molecules may experience and respond to cellular environments in distinct manners. The development and characterization of GEMs derived from encapsulin domain of Quasibacillus thermotolerans and having a diameter of about 50 nm, henceforth called “50 nm-GEMs,” is described herein, to gauge the biophysical properties of cells at a larger mesoscale size range.

In the present study, the encapsulin domain from Quasibacillus thermotolerans (“QtE”) was utilized, and a fusion protein was created with fluorescent tags at the C-terminal domain. Structural analysis shows that the encapsulin self-assembles into a 240-subunit icosahedral compartment with a diameter of 42 nm (Tobias W Giessen et al., Large protein organelles form a new iron sequestration system with high storage capacity. eLife, 8: e46070, 2019). A fusion protein was created herein, with a fluorescent tag attached to the C-terminal domain of the encapsulin ORF via a GS-linker. In addition, a FLAG-tag sequence was implemented to the C-terminus of the fluorescent tag via a second GS linker to utilize FLAG-peptide purification of GEMs for cryoEM structural analysis. Purification of GEMs from Saccharomyces cerevisiae via the FLAG-peptide method, and subsequent cryoEM imaging, revealed a final encapsulin diameter of 51-nm.

When expressed in Saccharomyces cerevisiae and mammalian cells, these particles formed bright and stable fluorescent nanoparticles that have been tracked via single particle tracking to assess their effective diffusion in a comparative analysis with 40-nm GEMs derived from Pyrococcus furiosis (PfV) encapsulin. Data shows that 50-nm GEMs of the present invention have a lower effective diffusion coefficient than the 40-nm PIV GEMs, owing to the 10-nm diameter difference. This observation holds true in both Saccharomyces cerevisiae and mammalian cells in which GEMs are expressed. Thus, the 50-nm GEMs of the present invention allow investigating the intracellular environment at a larger mesoscale length, providing information about the effects of macromolecular crowding on complexes of greater sizes.

It was shown herein that polysome collapse and condensation of RNA transiently reduces elastic confinement in the cytoplasm; coarse grained molecular dynamic simulations support this as a minimal mechanism for the observed biophysical changes. Increased mesoscale diffusivity correlates with the efficient formation of Q-bodies, membraneless organelles that compartmentalize misfolded peptides during stress. Synthetic, light-induced RNA condensation also fluidized the cytoplasm. Together, the present disclosure study revealed a functional role for stress-induced translation inhibition and formation of RNP condensates in modulating the physical properties of the cytoplasm to enable efficient response of cells to stress conditions.

Further herein, mesoscale particle diffusivity was investigated in the period immediately following acute stresses in both yeast and mammalian cells. A transient increase in mesoscale cytoplasmic diffusivity was discovered in all stress conditions tested. The collapse of polysome complexes was a prerequisite for this fluidization. However, polysome collapse was not sufficient; in addition, the condensation of released mRNA into P-bodies or stress granules was also required. In yeast cells, this fluidization of the cytoplasm was required for the efficient formation of Q-body condensates. These findings provide new insights into how polysome collapse and RNA condensation influence the biophysical properties of cells, allowing for efficient mesoscale reorganization of the cell.

### Definitions

As used herein the terms “50 nm GEMs”, “50 nm-GEMs,” and “GEMs having a diameter of about 50 nm” are used interchangeably to refer to genetically encoded multimeric nanoparticles (GEMs) having a diameter of about 50 nm, wherein the term “about” is within 10% of the described value. In some embodiments of the invention, the 50 nm-GEMs of the invention have a diameter of 45-55 nm, or 47-53 nm, or 48-52 nm, or 51 nm.

The terms “a,” “an,” and “the” do not denote a limitation of quantity, but rather denote the presence of “at least one” of the referenced item.

The terms “patient,” “individual,” “subject,” “mammal,” and “animal” are used interchangeably herein and refer to mammals, including, without limitation, human and veterinary animals (e.g., cats, dogs, rabbits, cows, horses, sheep, pigs, etc.) and experimental animal models. In a preferred embodiment, the subject is a human.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

### Polynucleotides and Polypeptides

Provided herein is an isolated polynucleotide encoding a fusion polypeptide comprising an encapsulin domain from Quasibacillus thermotolerans (“QtE”) and a fluorescent tag, wherein the fluorescent tag is located to the C-terminus of the QtE.

In certain embodiments, QtE comprises an amino acid sequence SEQ ID NO: 1 or a functional fragment thereof or a sequence that has at least 90% amino acid sequence identity thereto. In certain embodiments, QtE comprises the amino acid sequence SEQ ID NO: 1. In certain embodiments, QtE consists of the amino acid sequence SEQ ID NO: 1.

In certain embodiments, the fluorescent tag is selected from red fluorescent protein mScarlet, green fluorescent protein (GFP), green fluorescent protein variant Sapphire, self-labeling HaloTag, and functional fragments and derivatives thereof. In certain embodiments, the fluorescent tag is Sapphire which comprises an amino acid sequence SEQ ID NO: 3. In certain embodiments, Sapphire consists of the amino acid sequence SEQ ID NO: 3. In certain embodiments, the fluorescent tag is GFP which comprises an amino acid sequence SEQ ID NO: 5. In certain embodiments, GFP consists of the amino acid sequence SEQ ID NO: 5. In certain embodiments, the fluorescent tag is HaloTag which comprises an amino acid sequence SEQ ID NO: 8. In certain embodiments, HaloTag consists of the amino acid sequence SEQ ID NO: 8. In certain embodiments, the fluorescent tag is mScarlet which comprises an amino acid sequence SEQ ID NO: 10. In certain embodiments, mScarlet consists of the amino acid sequence SEQ ID NO: 10.

In certain embodiments, the fluorescent tag is attached to the C-terminus of the QtE via a linker. In certain embodiments, the linker is a GS-containing linker. In certain embodiments, the linker comprises an amino acid sequence SGSGSGSGSGSG (SEQ ID NO: 2). In certain embodiments, the linker comprises an amino acid sequence GGSGGSGSGGSG (SEQ ID NO: 4).

In certain embodiments, the fusion polypeptide further comprises a purification tag. In certain embodiments, the purification tag is located to the C-terminus of the fluorescent tag. In certain embodiments, the purification tag is a FLAG tag. In certain embodiments, the FLAG tag comprises an amino acid sequence SEQ ID NO: 6. In certain embodiments, the purification tag is attached to the C-terminus of the fluorescent tag via a linker. In certain embodiments, the linker is a GS-containing linker. In certain embodiments, the linker comprises an amino acid sequence SGSGSGSGSGSG (SEQ ID NO: 2). In certain embodiments, the linker comprises an amino acid sequence GGSGGSGSGGSG (SEQ ID NO: 4).

In certain embodiments, the fusion polypeptide further comprises a sequence from the PopZ protein from Caulobacter crescentus, wherein the PopZ sequence is located to the N terminus of QtE and comprises the C-terminal helical oligomerization domain of PopZ or a functional fragment thereof or a sequence that has at least 90% amino acid sequence identity thereto. In certain embodiments, the sequence from the PopZ protein comprises an amino acid sequence SEQ ID NO: 9. In certain embodiments, the sequence from the PopZ protein consists of the amino acid sequence SEQ ID NO: 9.

In certain embodiments, upon its expression in a host cell the fusion polypeptide forms genetically encoded multimeric nanoparticles (GEMs) having a diameter of about 50 nm. In certain embodiments, upon its expression in a host cell the fusion polypeptide forms GEMs having a diameter of 45-55 nm. In certain embodiments, upon its expression in a host cell the fusion polypeptide forms GEMs having a diameter of 47-53 nm. In certain embodiments, upon its expression in a host cell the fusion polypeptide forms GEMs having a diameter of 48-52 nm. In certain embodiments, upon its expression in a host cell the fusion polypeptide forms GEMs having a diameter of 51 nm. In certain embodiments, the host cell is a yeast cell. In certain embodiments, the host cell is a mammalian cell.

In certain embodiments, the fusion polypeptide comprises the amino acid sequence SEQ ID NO: 11. In certain embodiments, the polynucleotide encoding said fusion polypeptide comprises the nucleotide sequence SEQ ID NO: 17.

In certain embodiments, the fusion polypeptide comprises the amino acid sequence SEQ ID NO: 12. In certain embodiments, the polynucleotide encoding said fusion polypeptide comprises the nucleotide sequence SEQ ID NO: 18.

In certain embodiments, the fusion polypeptide comprises the amino acid sequence SEQ ID NO: 13. In certain embodiments, the polynucleotide encoding said fusion polypeptide comprises the nucleotide sequence SEQ ID NO: 19.

In certain embodiments, the fusion polypeptide comprises the amino acid sequence SEQ ID NO: 14. In certain embodiments, the polynucleotide encoding said fusion polypeptide comprises the nucleotide sequence SEQ ID NO: 16.

In certain embodiments, the fusion polypeptide comprises the amino acid sequence SEQ ID NO: 15. In certain embodiments, the polynucleotide encoding said fusion polypeptide comprises the nucleotide sequence SEQ ID NO: 20.

In certain embodiments, the fusion polypeptide comprises the amino acid sequence SEQ ID NO: 23. In certain embodiments, the polynucleotide encoding said fusion polypeptide comprises the nucleotide sequence SEQ ID NO: 21.

In certain embodiments, the fusion polypeptide comprises the amino acid sequence SEQ ID NO: 24. In certain embodiments, the polynucleotide encoding said fusion polypeptide comprises the nucleotide sequence SEQ ID NO: 22.

In certain embodiments, the fusion polypeptide comprises the amino acid sequence SEQ ID NO: 26. In certain embodiments, the polynucleotide encoding said fusion polypeptide comprises the nucleotide sequence SEQ ID NO: 25.

Further provided herein is a fusion polypeptide encoded by any of the polynucleotides described herein.

### Genetically Encoded Multimeric Nanoparticles

Additionally provided herein is a genetically encoded multimeric nanoparticle (GEM) comprising the fusion polypeptide as described above. In certain embodiments, the GEM has a diameter of about 50 nm. In certain embodiments, the GEM has a diameter of 45-55 nm. In certain embodiments, the GEM has a diameter of 47-53 nm. In certain embodiments, the GEM has a diameter of 48-52 nm. In certain embodiments, the GEM has a diameter of 51 nm.

Additionally provided herein is a method of producing genetically encoded multimeric nanoparticles (GEMs) comprising:
a) incubating a host cell as described herein under conditions when the fusion polypeptide is produced and assembles into GEMs, and
b) isolating the GEMs.

### Fluorescent Tags

In some embodiments, the fluorescent tag is selected from red fluorescent protein mScarlet, green fluorescent protein (GFP), green fluorescent protein variant Sapphire, self-labeling HaloTag, and functional fragments and derivatives thereof.

In some embodiments, the fluorescent tag comprises, but is not limited to, a cyan fluorescent protein (e.g., ECFP, ECFP2, TagCFP, mTagCFP2, mKalama1, SCFP3C, Cerulean, mTurquoise, mTurquoise2, Azurite, Midoriishi-Cyan, mTFP1), a green fluorescent protein (e.g., GFP, EGFP (Enhanced GFP), Emerald, Sapphire, T-Sapphire, Superfolder GFP (sfGFP), Azami Green, mWasabi, ZsGreen, TagGFP, TagGFP2, TurboGFP, CopGFP, AceGFP, Clover, mUKG, mNeonGreen), a yellow fluorescent protein (e.g., YFP, EYFP (Enhanced YFP), Topaz, Venus, Citrine, YPet, SYFP, mAmetrine, TagYFP, TurboYFP, ZsYellow, PhiYFP, SYFP2), an orange fluorescent protein (e.g., Kusabira Orange, Kusabira Orange2, Monomeric Kusabira-Orange, mOrange, mOrange2, mKOK, mKO2, dTomato, dTomato-Tandem), a red fluorescent protein (e.g., RFP, mRuby, mRuby2, mApple, mStrawberry, AsRed2, mRFP1, JRed, mCherry, eqFP611, tdRFP611, HcRed1, mRaspberry, tdTomato, TurboRFP, TagRFP, TagRFP-T, mScarlet, mTangerine, mPlum, HcRed-Tandem, mKate, mKate2, Katushka, mNeptune), a far-red or near-infrared fluorescent protein (e.g., NirFP, TagRFP657, IFP1.4, iRFP, tdRFP639, AQ143), a blue fluorescent protein (e.g., BFP, EBFP (Enhanced BFP), Azurite, SBFP2 (Strongly Enhanced BFP), EBFP2, Sirius, mTagBFP), or a specialized photoactivatable, photoconvertible, and/or photoswitchable fluorescent protein (e.g., PA-GFP, PS-CFP2, PA-mRFP1, PA-mCherry1, Phamret, Kaede, wtKikGR, mKikGR, wtEosFP, dEos, mEos2, Dendra2, Dronpa, Dronpa-3, rsFastLine, Padron, bsDronpa, KFP1, E2GFP, rsCherry, rsCherryRev, IrisFP).

The method used to detect the fluorescent tag(s) may include, but is not limited to, fluorescence microscopy, flow cytometry, spectrofluorometry, confocal microscopy, fluorescence-activated cell sorting (FACS), fluorescence resonance energy transfer (FRET), total internal reflection fluorescence (TIRF) microscopy, fluorescence lifetime imaging microscopy (FLIM), and/or multiphoton microscopy. The method may be selected based on the specific requirements of the experiment, such as the resolution needed, the depth of penetration, and the type of sample being analyzed.

In some embodiments, the fluorescent tag is Sapphire which comprises an amino acid sequence SEQ ID NO: 3.

In some embodiments, the fluorescent tag is GFP which comprises an amino acid sequence SEQ ID NO: 5.

In some embodiments, the fluorescent tag is HaloTag which comprises an amino acid sequence SEQ ID NO: 8.

In some embodiments, the fluorescent tag is mScarlet which comprises an amino acid sequence SEQ ID NO: 10.

### Vectors

In some embodiments, the vector is a mammalian expression vector.

In some embodiments, the mammalian expression vector includes, but is not limited to the pCMV series, the pcDNA series, the pEF series, the pIRES series, the pFLAG-CMV series, the pTRE series, the pGL4 series, the pBABE series, pZeoSV2, and pLXSN. In certain embodiments, the polynucleotide encoding the fusion polypeptide is operably linked to a mammalian Ubiquitin C (UBC) promoter.

In some embodiments, the vector is a viral vector.

In some embodiments, the vector is a lentiviral vector.

In some embodiments, the lentiviral vector includes, but is not limited to pLenti-CMV, pLKO.1, pLVX, pLenti6/V5, pLentiCRISPR, pLenti-Puro, pLenti-X, pUBC, and pLenti-GFP. In some embodiments, the vector is a yeast expression vector.

In some embodiments, the yeast expression vector includes, but is not limited to, pYES2, the pPICZ series, the pGAPZ series, the pRS series, the pESC series, the pTEF series, the pYEX series, the pYAC series, the pFBD series, and the pUG series.

In some embodiments, the yeast expression vector is a yeast integration expression vector. In certain embodiments, the polynucleotide encoding the fusion polypeptide is operably linked to a yeast transcription factor (INO4) promoter or a yeast histidine synthesis (His3) promoter.

The choice of expression vector may depend on various factors such as the host cell type, desired expression level, and post-translational modifications.

### Host Cells

In certain embodiments, the host cell is a yeast cell.

In certain embodiments, the host cell is a mammalian cell. In certain embodiments, the host cell is a cell from a veterinary animal or an animal model. In certain embodiments, the host cell is a human cell.

In some embodiments, the host cell may include, but is not limited to, a bacterial cell (e.g., Escherichia coli), a yeast cell (e.g., Saccharomyces cerevisiae, Pichia pastoris), a mammalian cell (e.g., HEK293 (Human Embryonic Kidney 293), CHO (Chinese Hamster Ovary), or HeLa), an insect cell (e.g., Sf9, Sf21), and a plant cell (e.g., Nicotiana benthamiana).

The choice of host cell may depend on various factors such as the type of protein being expressed, the need for specific post-translational modifications, the scale of production, and the case of genetic manipulation.

### Methods

The GEMs as described herein may be used to detect several various biophysical consequences via their use as probes for sensing biophysical changes in the intracellular environment. GEMs are highly sensitive to the physical consequences of such biophysical changes.

The biophysical properties that may be probed (e.g., measured or inferred) by GEMs may include cytoplasmic fluidity, effective diffusivity, anomalous diffusion exponent, viscoelastic/mechanical state, cytoplasmic crowding, spatial heterogeneity, confinement/caging, non-equilibrium/active fluctuation, phase separation and condensates, and any combination thereof.

Effective diffusivity (D) may be probed via the average case of GEM motion as crowding and viscosity correspond to a lower D. An about 2-fold change in GEM motion is observed when ribosomes are modulated (Delarue, M. et al., mTORC1 Controls Phase Separation and the Biophysical Properties of the Cytoplasm by Tuning Crowding, Cell, 2018, 174 (2), 338-349.c20; Garner, R. M. et al., Vast heterogeneity in cytoplasmic diffusion rates revealed by nanorheology and Doppelgänger simulations, Biophysical Journal, 2023, 122 (5), 767-783).

Anomalous diffusion exponent (a) may be probed via GEMs as an α<1 indicates subdiffusion due to viscoelasticity or obstruction. An about 2-fold change in GEM motion is observed when ribosomes are modulated (Delarue, M. et al., mTORC1 Controls Phase Separation and the Biophysical Properties of the Cytoplasm by Tuning Crowding, Cell, 2018, 174 (2), 338-349.e20; Garner, R. M. et al., Vast heterogeneity in cytoplasmic diffusion rates revealed by nanorheology and Doppelgänger simulations, Biophysical Journal, 2023, 122 (5), 767-783). MSD scaling may be used to infer a.

Viscoelastic/mechanical state may be probed via GEMs as MSD and autocorrelation analysis reveal fluid-like vs. gel-like behavior. Microtubule perturbation reduces diffusion (Carlini, L., et al, Microtubules Enhance Mesoscale Effective Diffusivity in the Crowded Metaphase Cytoplasm, Dev Cell, 2020, 54 (5), 574-582.c4).

Cytoplasmic crowding may be probed via GEMs as changes in large macromolecule content alter GEM mobility. A change in GEM mobility is observed upon ribosome-driven crowding (Delarue, M. et al., mTORC1 Controls Phase Separation and the Biophysical Properties of the Cytoplasm by Tuning Crowding, Cell, 2018, 174 (2), 338-349.e20).

Spatial heterogeneity may be probed via GEMs as mapping D or a can reveal compartments, gradients, and structural domains. GEMs have been shown to probe spindle versus cytoplasm effects (Carlini, L., et al, Microtubules Enhance Mesoscale Effective Diffusivity in the Crowded Metaphase Cytoplasm, Dev Cell, 2020, 54 (5), 574-582.c4).

Confinement/caging may be probed via GEMs as GEMs may be trapped by cytoskeletal or condensate barriers. Microtubule disruption in metaphase can reduce D by about 30%.

Non-equilibrium/active fluctuation may be probed via GEMs as excess motion beyond thermal effects reflects ATP-driven activity (Delarue, M. et al., mTORC1 Controls Phase Separation and the Biophysical Properties of the Cytoplasm by Tuning Crowding, Cell, 2018, 174 (2), 338-349.c20; Parry, B. R. et al., The Bacterial Cytoplasm Has Glass-like Properties and Is Fluidized by Metabolic Activity, Cell, 2014, 156 (1), 183-194).

Phase separation and condensates may be probed via GEMs as GEMs slow down or are excluded from dense liquid phases. Ribosome crowding promotes phase separation (Delarue, M. et al., mTORC1 Controls Phase Separation and the Biophysical Properties of the Cytoplasm by Tuning Crowding, Cell, 2018, 174 (2), 338-349.c20).

In some embodiments, a change in the biophysical property results in a biophysical consequence.

The biophysical consequence may include ATP depletion or metabolic collapse, pH changes, glucose starvation, a change in ribosome concentration and/or polysome disassembly, formation of RNA condensates (P-bodies, stress granules, RNP granules), free RNA abundance, assembly of Q-bodies, and any combination thereof.

ATP depletion or metabolic collapse reduces active transport and cytoplasmic “stirring” and thus increases confinement or viscosity (Parry, B. R. et al., The Bacterial Cytoplasm Has Glass-like Properties and Is Fluidized by Metabolic Activity, Cell, 2014, 156 (1), 183-194).

pH changes alter protein charge/solubility, potentially promoting condensate formation or cytoskeletal rearrangements.

Glucose starvation causes ATP drop and stress granule formation, increasing crowding and reducing fluidity, which is detectable as decreased GEM mobility (Knapp, B. D. et al., Translating the Physical Code of Life, Cell, 2018, 174 (2), 253-255).

A change in ribosome concentration and/or polysome disassembly is a major contributor to mesoscale crowding. Depolymerizing polysomes or reducing ribosome numbers (via mTORC1 inhibition) increases GEM diffusivity (Delarue, M. et al., mTORC1 Controls Phase Separation and the Biophysical Properties of the Cytoplasm by Tuning Crowding, Cell, 2018, 174 (2), 338-349.c20).

Formation of RNA condensates (P-bodies, stress granules, RNP granules) are detected via GEMs as dense structures trap GEMs or slow them locally, increasing heterogeneity in diffusion (Xie, Y. et al., Polysome collapse and RNA condensation fluidize the cytoplasm, Molecular Cell, 2024, 84 (14), 2698-2716.c9).

Free RNA abundance is detected as elevated levels of free RNA abundance contribute to crowding, whereas depletion “decrowds” the cytoplasm and increases GEM mobility.

Assembly of Q-bodies may be detected as these protein condensates act as diffusion barriers or load reservoirs, altering mesoscale fluidity (Xie, Y. et al., Polysome collapse and RNA condensation fluidize the cytoplasm, Molecular Cell, 2024, 84 (14), 2698-2716.c9).

### EXAMPLES

The following examples are provided to further describe some of the embodiments disclosed herein. The examples are intended to illustrate, not to limit, the disclosed embodiments.

### Materials and Methods

### Yeast Strains

All S. cerevisiae strains constructed for and used in the present disclosure are listed in Table 1. Unless otherwise stated, strains were grown at 30° C. in Synthetic Complete Media. Yeast strains were constructed using standard molecular genetic methods, and verified by fluorescence microscopy for GEM expression.

TABLE 1
Yeast Strain Information

| | Genetic | |
|---|---|---|
| Strain ID | Background | Modification |
| LH4501 | BY4741 | pINO4::pINO4-Pfv-Sapphire-LEU2 |
| LH4580 | BY4741 | pINO4::pINO4-QtE-Sapphire-LEU2 |
| LH4832 | BY4741 | pHis3::pHis3-QtE-GFP-FLAG-URA3 |
| LH4348 | BY4741 | pINO4::pINO4-PfV-GFP-FLAG-LEU2 |

Saccharomyces cerevisiae BY4741 and W303 strains were grown in synthetic complete media+2% dextrose (SCD) according to standard Cold Spring Harbor Protocols unless otherwise stated. Exponentially growing cultures between O.D. 600 0.1 and 0.4 were used in all experiments unless otherwise noted.

Construction of 50 nm-GEM and 40 nm-GEM Plasmids

The open reading frame (ORF) encoding the Quasibacillus thermotolerans (QtE) encapsulin protein based on the published crystal structure (www[dot]rcsb[dot]org/structure/6NJ8) was codon optimized for yeast expression and synthesized as an IDT gene block (www[dot]idtdna[dot]com/pages). The 50 nm-GEM yeast expression vector was constructed by Gibson assembly into a pRS305 vector, with fusion of the 5′ end of the QtE-encapsulin ORF to a homologous sequence of the yeast INO4 promoter, and at the 3′ end, via a 6×Ser-Gly linker (SEQ ID NO: 2), with the ORF of the T-Sapphire fluorophore (pLH1976: pRS305-PINO4-QtE-GS-Sapphire). The mammalian expression vector was assembled by subcloning the QtE-GS-T-Sapphire gene cassette into a pLVX lentiviral backbone, with transcription directed by the mammalian pUBC promoter.

Initial versions of the 50 nm-GEMs did not assemble efficiently. To increase assembly efficiency, a sequence from the PopZ protein from Caulobacter crescentus was appended to the N-terminus of the 50 nm-GEM protein.

For insertion of the FLAG tag, the FLAG and GFP sequence were cloned together with the GFP sequence into a yeast expression vector containing the scaffold sequence for 50 nm-GEMs (QtE) by Gibson Assembly. The plasmid was stored in bacteria glycerol stocks. Plasmids constructed for and used in the present disclosure are listed in Table 2.

TABLE 2
Plasmid Information

| Plasmid | | |
|---|---|---|
| ID | | Seq ID NO: |
| pLH497 | pRS305-pINO4-PfV-GS-Sapphire | SEQ ID NO: 23 |
| pLH1976 | pRS305-pINO4-QtE-GS-Sapphire | SEQ ID NO: 14 |
| pLH1984 | pUBC-PfV-6XSG-TSapphire | SEQ ID NO: 26 |
| pLH2220 | pUBC-popZ3H-GS-QtE-6xSG-TSapphire | SEQ ID NO: 11 |
| pLH2328 | pUBC-popZ3H-GS-QtE-6xSG-mScarlet | SEQ ID NO: 13 |
| pLH2494 | pUBC-popZ3H-GS-QtE-6xSG-Halo | SEQ ID NO: 12 |
| pLH2508 | pRS306-pHis3-QtE-GS-GFP-GS-FLAG | SEQ ID NO: 15 |
| pLH2608 | pRS305-pINO4-PfV-GS-Sapphire-GS- | SEQ ID NO: 24 |
| | FLAG | |

### Mammalian Cell Culture

HeLa cells were used in the present disclosure. HeLa cells were grown in DMEM (Gibco, Cat. No. 11995073) supplemented with 10% FBS (Gemini bio-products, Cat. no. 100-106), 2 mM L-Glutamine (Gibco, Cat. No. 25030-081) and Penstrep (Gibco, Cat. No. 15140-122). All cells were grown in a humidified incubator at atmospheric 37° C. and 5% CO₂. Transient transfection was achieved using FuGENE® HD Transfection Reagent (Promega, Cat. No. E2311), and OptiMEM Reduced Serum Media (Gibco, Cat. No. 31985070). Transfection grade plasmid DNA was extracted using ZymoPURE™ Plasmid Miniprep Kit (Zymo, Cat. No. 224164). Fugene was allowed to reach room temperature and briefly inverted prior to each use. 200 μL of OptiMEM was aliquoted into a sterile Eppendorf tube, along with 1 μL Fugene per 0.5 ug of DNA. 0.5 ug of DNA yielded optimum GEM expression in Hela cells, and was utilized as the standard DNA transfection concentration in all experiments. Upon adding DNA and transfection reagent to OptiMEM, the mixture was vortexed for 5 seconds, spun down for 5 seconds, and incubated at room temperature for 10 minutes. The mixture was then added dropwise to HeLa cells plated on glass bottom dishes (Cellvis, Cat. No. P06-1.5H-N). After 24 hours, a full media change to DMEM was conducted, and cells were imaged for GEM expression on a confocal microscope.

Affinity-Purification of 50 nm-GEMS from S. cerevisiae

A method for the isolation of endogenous whole NPCs from S. cerevisiae (PMID: 35412228) was adapted to purification of natively expressed GEMs. Briefly, strains harboring FLAG-tagged 50 nm-GEMs and 40 nm-GEMs incorporated at the LEU2 locus were grown in YPD media at 30° C. until mid-log phase (˜3×10⁷ cells/ml), harvested, frozen in liquid nitrogen and cryogenically lysed in a planetary ball mill PM 100 (Retsch) (lab[dot]rockefeller[dot]edu/rout/protocols). Affinity purification was performed in resuspension buffer (20 mM HEPES/KOH pH 7.4, 500 mM sodium chloride, 0.1% (w/v) Triton X-100, 0.1% (w/v) Tween-20, 1 mM DTT, 10% (v/v) glycerol, 1/500 (v/v) Protease Inhibitor Cocktail (Sigma)) and native elution by protease cleavage was achieved in a similar buffer without Triton X-100 or protease inhibitors. Protease inhibitors were added to the purified NPCs and either conserved at 4° C. or frozen in liquid nitrogen and stored at −80° C.

Affinity captured NPCs were eluted from beads by addition of 20 μl of 1×LDS (lithium dodecyl sulfate) loading buffer (Thermo Fisher) and vortexing for 10 minutes at room temperature. Eluted NPCs were run on an NuPAGE 4-12% gel for 60 minutes and analyzed by Coomaisse.

### Microscopy

For imaging yeast, cells were cultured to log phase at OD600 0.4 in synthetic complete media (2% Dextrose). 384 glass-bottom plates were coated with Concanavalin-A (ConA). Using a Nikon Ti Eclipse Microscope equipped with a Hamamatsu camera, frames were captured every 10 ms over a duration of 400 frames. Fluorescence was observed using lasers.

### Software and Algorithms

The following software and algorithms were employed in the analysis: ImageJ, Fiji, Mosaic, Cellpose, and GEMspa (8). The code used in this study is available at github[dot]com/liamholtlab (GEMs Analysis Tools, GitHub).

### Human Cell Lines

U2OS cells were cultured in DMEM containing high glucose and sodium pyruvate supplemented with 10% FBS and 50 U/ml penicillin, and 50 μg/ml streptomycin (Gibco™, Cat. No. 15140122), and maintained at 37° C. in a humidified incubator with 5% CO₂. Cells were regularly split in fresh medium upon reaching 80-90% confluency. All cells were routinely tested for mycoplasma by PCR screening of the conditioned medium.

### Yeast Strain Construction

Endogenous protein tagging at the C terminal with fluorescent protein was constructed via transformation of PCR products containing fluorescent proteins with auxotrophic marker gene, using either plasmid pLH1688 or pLH1532. The PCR products contained 42 bp of homology to the 5′ and 3′ of the target gene stop codon region. Deletion strains were constructed via transformation with PCR products containing antibiotic resistance cassettes (pLH1493 and pLH1494). PCR products contained 42 bp of homology to the 5′ and 3′ genomic regions immediately adjacent to the gene to be deleted. The scarless Pab1 P-domain (419-502 amino acid) deletion strain was generated following the protocol outlined in (117). Briefly, WT W303 yeast cells were first transformed with a URA3 expression cassette replacing the Pab1 P-domain, which contained flanking DNA and included a stop codon at the beginning of the cassette. The resulting intermediate strain was selected on SCD-Ura plates and confirmed through colony PCR and sequencing. Subsequently, this intermediate strain was transformed with a synthesized dsDNA containing only genes upstream and downstream of P-domain, excluding the P-domain itself. Transformants were selected on plates containing 5-FOA. Single colonies of scarless Pab1 P-domain deletion were isolated and verified using colony PCR and sequencing. To integrate either 40 nm-GEMs or μNS particles into the yeast strain, plasmid pLH497 was linearized by restriction enzyme SnaBI, and pLH1125 was linearized by primers located at the HIS3 promoter region. To integrate the ATP sensor QUEEN or pH sensor pHluorin, plasmid pLH2156 was linearized by restriction enzyme PstI and plasmid pLH1097 was linearized by restriction enzyme KasI. All DNA products were transformed to the yeast strains based on a lithium acetate approach according to standard protocol.

### Lentivirus Production and Cell Transduction

HEK293T cells (9×10⁶ per 15 cm dish) were plated in antibiotic free DMEM (Gibco, Cat. No. 11995073) supplemented with 10% FBS (Gemini bio-products, Cat. no. 100-106). The next day, cells were transfected with transgene plasmid (pLH1984) together with lentivirus packaging plasmids psPAX2 and pMD2.G, using fuGENE HD™ transfection reagent (Promega, Cat. no. E2312) following manufacturer's protocol. 24 hours later, 15 ml antibiotic free DMEM was replaced and the supernatants were collected at both 48 and 72 h post-transfection, and stored at 4° C. Virus titers were concentrated by centrifugation at 4,000 rcf for 40 minutes in an Amicon Ultra-15 30 KDa centrifugal filter (MilliporeSigma, Cat. No. UFC903024).

Concentrated viral suspensions were aliquoted and stored at −80° C. until later use. Lentivirus was introduced into U2OS wild type and G3BP1/2 dKO cell lines of interest via reverse transduction with 1-10 μL of concentrated virus in fresh media, and replacing media after 24 hours. After cell lines stabilized, they were frozen in 10% DMSO (Sigma-Aldrich, Cat. no. D2650-100) in FBS (Gemini bio-products, Cat. no. 100-106) and thawed for use in experiments whenever needed. Transient transfection of mammalian cell lines

For transfection, U2OS cells were seeded as 60-70% confluency in a 6-well glass bottom plate (Cellvis, Cat. No. P06-1.5H-N) on the day before transfection and were transfected with 1 μg of plasmid DNA (pLH1972 or pLH2126) or Poly (I:C) (long synthetic analog of dsRNA, InvivoGen) per well using FuGENE HD™ reagent per manufacturer guidelines. 24 hour post-transfection, fresh DMEM medium was replaced. Imaging experiments were usually carried out between 24 to 48-hour post-transfection.

### Yeast Cell Culture for Imaging

Saccharomyces cerevisiae strains were revived from −80° C. freezer on YPD plate for overnight growth. The next day, a patch of yeast cells were inoculated into 5-mL synthetic complete media with 2% glucose (SCD), and the cultures were grown at 30° C. in a rotating incubator for 5-6 hours without exceeding an O.D.600 of 0.4. Afterwards, the cultures were prepared as a few tubes with 4-6 times of 10× dilution, and cultured for an overnight growth in order to reach O.D.600 between 0.1-0.4 for the next day's imaging experiment. To perform acute glucose starvation, 96-well (Cellvis, Cat. no. P96-1.5H-Nor) or 384-well (Cellvis, Cat. no. P384-1.5H-Nor) glass bottom imaging plates were precoated with 1 mg/ml concanavalin A (Alfa Aesar, Cat. no. J61221) before applying the yeast cell culture. 10 min later when cells settled down to the bottom of the imaging plate, the culture medium was removed completely, and four additional washes of cells were performed with SCD medium, and then imaging was performed, recorded as the initial 0 min time point. Afterwards, SCD medium was removed, followed by four additional washes of the cell with treatment medium. Cells were then imaged at the correspondent time points afterwards.

To perform acute glucose starvation, SC medium supplemented with 2% sorbitol (to balance the osmotic pressure exerted by 2% glucose) was used. To perform amino acids starvation, SC medium supplemented with 2% glucose but without any amino acids was used. To induce oxidative stress, cells were treated with SC medium with 2% glucose and 0.3 mM H₂O₂. To perform ATP depletion experiment, SC medium supplemented with 2% sorbitol, pH adjusted as 5.5, and supplemented with 2-Deoxy-d-glucose and antimycin A in combination at 20 mM and 10 μM, respectively, was used.

To perform hypotonic shock in yeast cells, yeast cells were diluted at low density (around O.D.=0.0015) and cultured in synthetic complete with 500 mM Kcl (SCD+500 mM Kcl) overnight to reach log phase growth the next morning (O.D.<0.5). Cells were attached on a 96-well glass bottom plate with 1 mg/ml concanavalin A before switching to acute glucose starvation medium (SC medium supplemented with 2% sorbitol).

### Drug Treatments

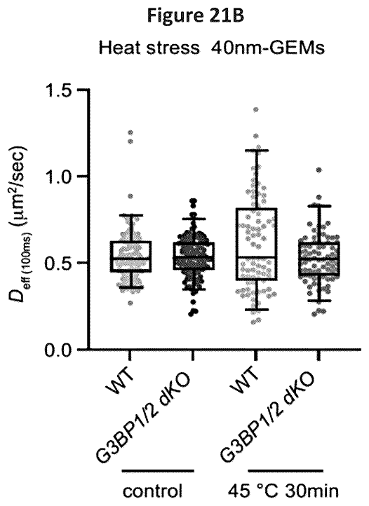

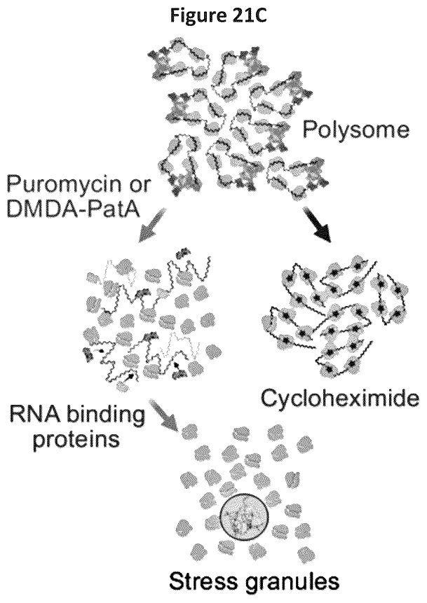

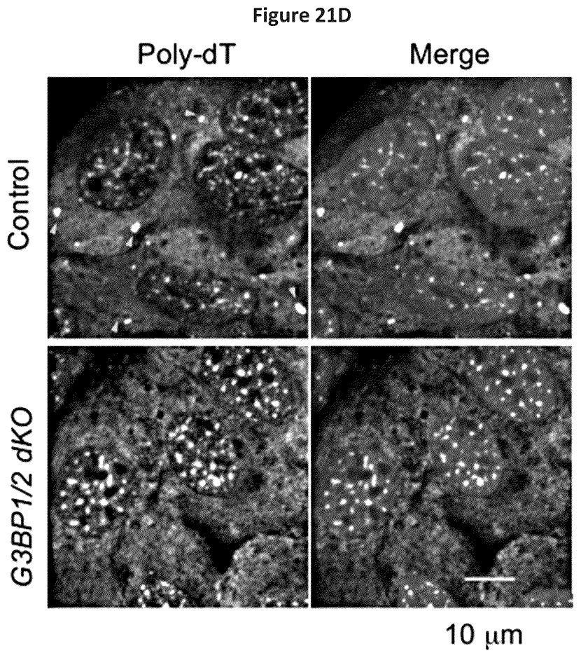

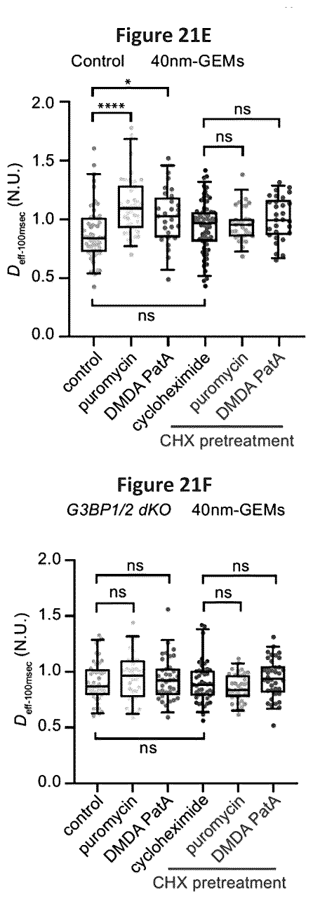

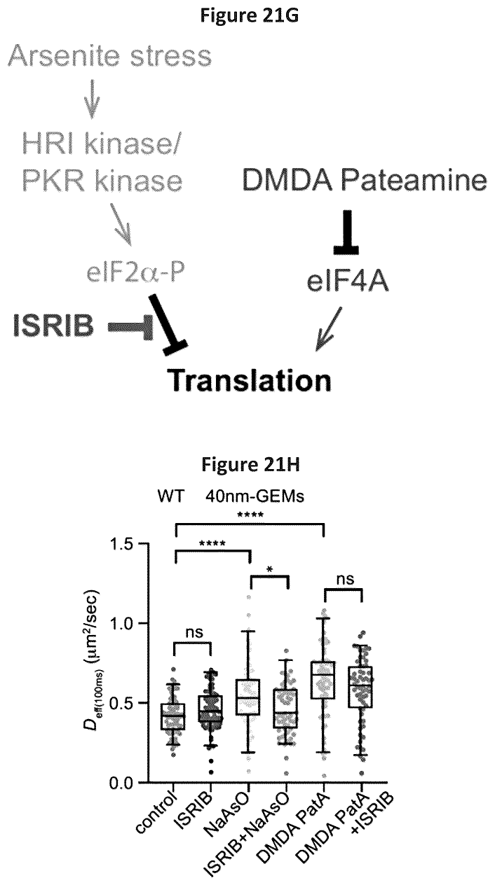

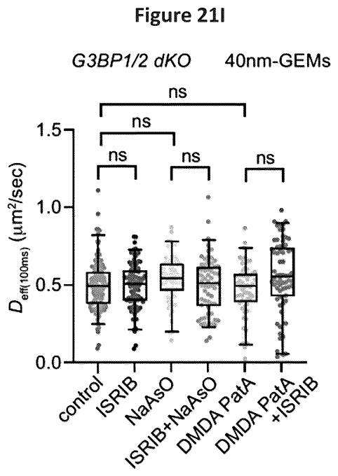

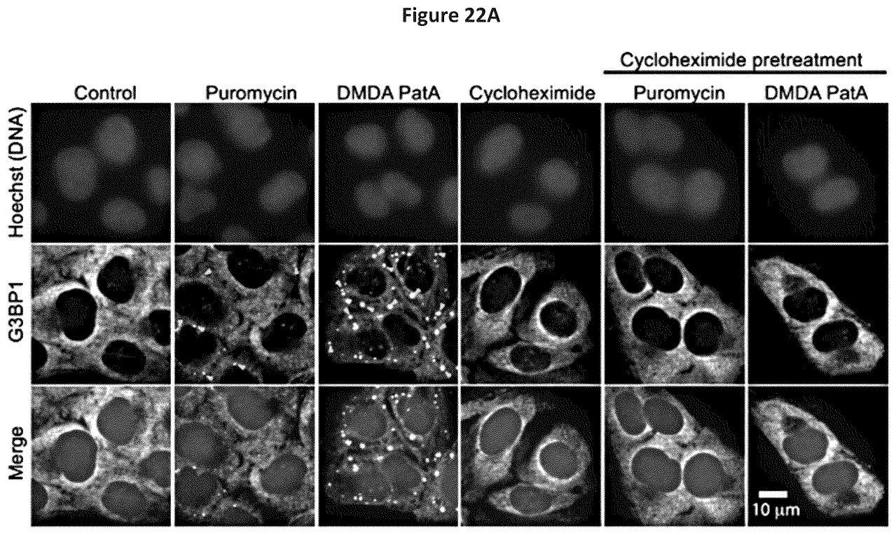

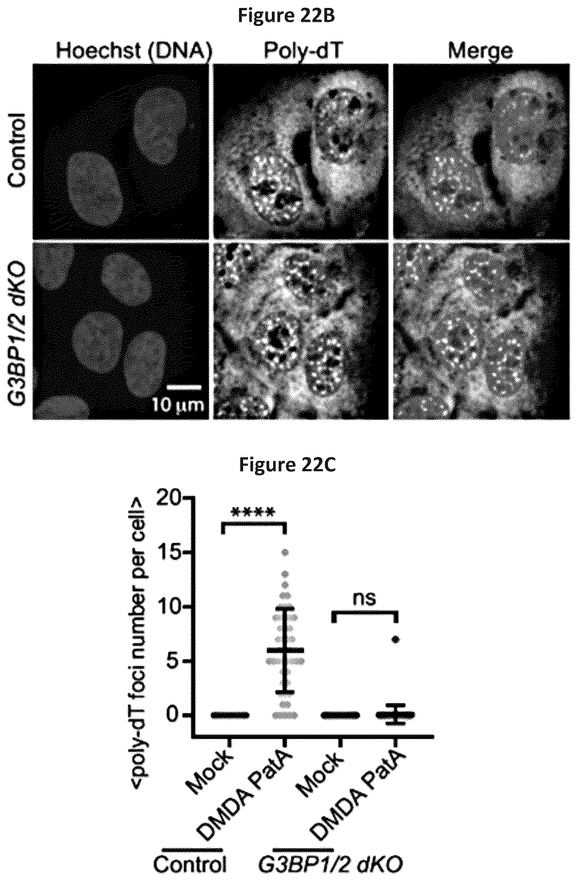

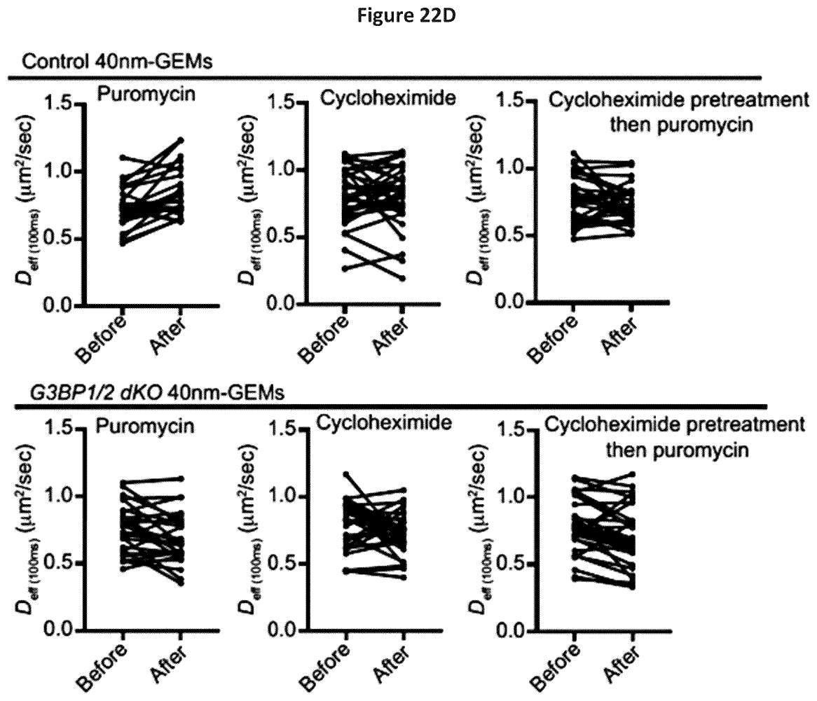

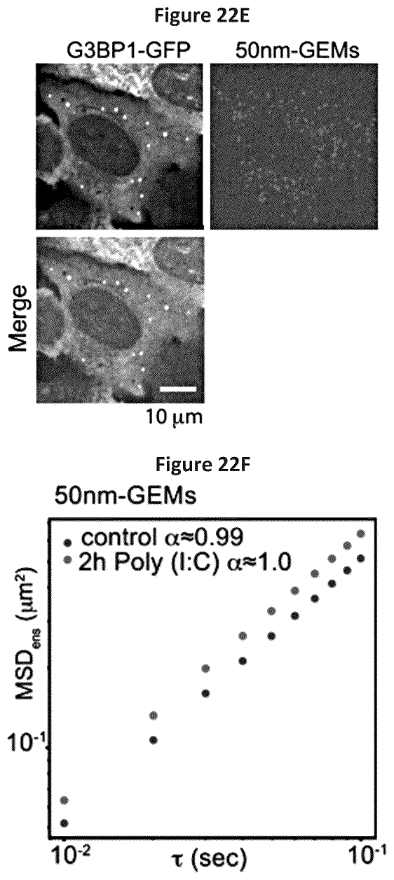

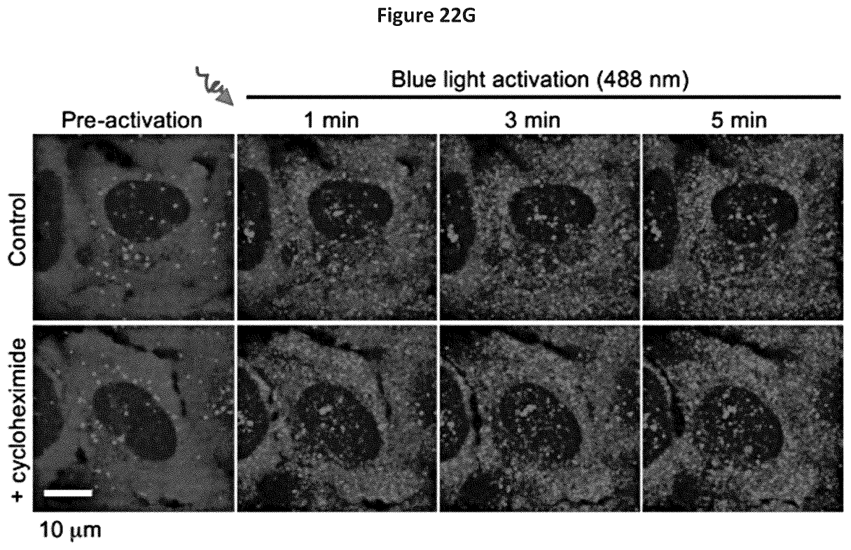

Cycloheximide was used at a final concentration of 100 μg/ml in all yeast experiments, while for mammalian cell experiments, 20 μg/ml of cycloheximide was used as a final concentration. Puromycin and des-methyl, des-amino pateamine A (DMDA PatA) were used at a final concentration of 100 μg/ml and 10 nM, respectively in all mammalian cell culture experiments. ISRIB was used at a final concentration of 200 nM in all mammalian cell culture experiments. Sodium arsenite was used at either 250 μM (FIGS. 21J-21K) or 100 μM (FIG. 22C) in mammalian cell culture experiments.

### Sucrose Gradient to Fractionate Polysomes

60 ml yeast cell culture in SCD medium reaching O.D.600 around 0.4 were collected by 3000 g centrifugation. Cells were washed with treatment medium before switching to the correspondent medium for additional 30 min culture, and 100 μg/ml cycloheximide was added to the culture before it was collected again by centrifugation and freshly frozen in a −80° C. freezer. To prepare sucrose gradient, four layers of sucrose solutions (2.7 ml each) were prepared in concentrations as 10%, 23.3%, 36.6% and 50%, where the sucrose were layered from bottom to top as high to low concentrations in 14×89 mm ultracentrifuge tubes (Beckman, 14×89 mm polypropylene centrifuge tubes, Cat. no. 331372), and each layer of sucrose was freshly frozen in −80° C. for 20 min before adding a second layer on top. To prepare cell lysis, polysome buffer (10 mM Tris, 10 mM MgCl₂, 100 mM KCl, 6 mM B-mercaptoethanol, 100 μg/ml cycloheximide, protease inhibitors (Thermo Scientific™, Cat. no. A32961) pH 7.5) were freshly prepared, and cell pellets were washed and then resuspended in 500 μl polysome buffer in the glass tube (Fisher Scientific, Cat. No. 14-961-32). Afterwards, 0.5 mm glass beads (BioSpec Products, Cat. No. 11079105) were added as half of the volume in cell suspension and vortexed four times as 30 sec each, with 1 min rest time on ice in between. Then all the cell lysis extracts were transferred to a new 1.5 ml tube, and 300 μl polysome buffer were used to wash the glass beads, then combined with the cell lysis extract. To clarify the cell lysis extract, 10 min of centrifugation at 15000 g was performed at 4° C. The supernatant of cell lysis extract was transferred to a new 1.5 ml tube, where the OD260 was measured by spectrophotometer. 9 units of OD260 cell lysis extract of each sample were then layered on top of the sucrose gradient and all the samples were then centrifuged at 39,000 rpm (Beckman, SW 41 Ti Swinging-Bucket Rotor) at 4° C. for 2.5 hrs. Afterwards, the samples were subjected to the fractionation system for analysis (Brandel, SYN-202 density gradient fractionation system).

### Passive Rheological Probes Imaging and Single Particle Tracking

To image 40 nm-GEMs in Saccharomyces cerevisiae, TIRF Nikon TI Eclipse microscope in highly inclined thin illumination mode (HILO) was used at GFP laser (49002-ET-EGFP) excitation with 100% power. Fluorescence was recorded with a scMOS camera (Zyla, Andor) with a 100× Phase, Nikon, oil NA=1.4 objective lens (part number=MRD31901, pixel size: 0.093 um). Cells were imaged at 100 Hz (10 ms per frame) for a total of 4 sec. To image μNS particles in Saccharomyces cerevisiae, Andor Yokogawa CSU-X confocal spinning disc on a Nikon Ti2 X1 microscope was used at 488 nm excitation with 10% power. Fluorescence was recorded with a sCMOS Prime 95B camera (Photometrics) with a 60× objective (pixel size: 0.18 μm), at a 100 ms image capture rate, with a time step for 1 min. To image 40 nm-GEMs in U2OS mammalian cell lines, Andor Yokogawa CSU-X confocal spinning disc on a Nikon Ti2 X1 microscope was used at 488 nm excitation with 100% power. Fluorescence was recorded with a sCMOS Prime 95B camera (Photometrics) with a 60× objective (pixel size: 0.18 μm), at a 10 ms image capture rate for a total of 2 sec.

The tracking of particles was performed with the Mosaic suite of FIJI (118), using the following parameters. For yeast 40 nm-GEMs: radius=3, cutoff=0, Per/Abs: variable, a link range of 1, and a maximum displacement of 7 px, assuming Brownian dynamics. For yeast μNS particles: radius=2, cutoff=0, Per/Abs: variable, a link range of 1, and a maximum displacement of 5 px, assuming Brownian dynamics. For mammalian cell 40 nm-GEMs: radius=2, cutoff=0, Per/Abs: variable, a link range of 1, and a maximum displacement of 5 px, assuming Brownian dynamics.

All trajectories were then analyzed with the GEM-Spa (GEM single particle analysis) software package that was developed by the inventors of the present disclosure: github[dot]com/liamholtlab/GEMspa/releases/tag/v0.11-beta (Keegan, S., 2021, GEMspa, Version 0.11 Beta, GitHub). Mean-square displacement (MSD) was calculated for every 2D trajectory, and trajectories continuously followed for more than 10 time points were used to fit with linear time dependence based on the first 10 time intervals to quantify time-averaged MSD: MSD (T)=4D_{eff}T, where T is the imaging time interval and D_{eff} is the effective diffusivity with the unit of μm²/s. To determine the ensemble-time-averaged mean-square displacement (MSD), all trajectories were fitted with MSD(τ)_{T-ens}=4Dτ^{α} where α is the anomalous exponent, with α=1 being Brownian motion, α<1 suggests sub-diffusive motion and α>1 as super-diffusive motion. To generate region of interest (ROI) for marking individual yeast cell, cellpose python package (github[dot]com/mouseland/cellpose; Stringer, C., Wang, T., Michaclos, M., & Pachitariu, M. (2021). Cellpose: a generalist algorithm for cellular segmentation. Nature Methods, 18, 100-106) was used to segment based on the bright field images or the average projection intensity of 40 nm-GEMs or μNS particles. These ROI were then input into GEM-Spa to quantify single cell effective diffusion, with at least three trajectories averaged for individual yeast cells. The median value of D_{eff} for single cell data was used to represent each condition.

For mammalian cell quantification of D_{eff}, the same field of the view of the cytosolic region covering the whole cell from individual cell was cropped before and after drug treatment, and the median value of D_{eff} from all trajectories within the cropped region were quantified. D_{eff} from the same cell after drug treatment was normalized to before drug treatment D_{eff} as a fold change value or all analyzed trajectories within one cell were averaged to present single cell D_{eff} for each condition.

Step sizes for all trajectories were extracted from GEM-Spa, to reflect the distance (with the unit of μm) of particles traveled for a given time interval: 100 ms for 40 nm-GEMs and different time intervals for μNS particles. To measure the average intensity of μNS particles from GEM-Spa, a fixed radius (radius=3) was used along the movie series, and the mean intensities of particles were measured at all the tracked frames and then summarized as average mean intensity.

Normalized velocity autocorrelation was analyzed using a custom-developed MATLAB (R2019a) program based on trajectories extracted from either experimental data or simulations (44). Initially, velocities within specific time intervals were calculated for each trajectory, resulting in velocity time series along either the x or y directions. Subsequently, autocorrelation functions were applied to these velocity time series, generating outputs as a function of various time delays. Due to the orthogonality, the autocorrelation functions from both x and y direction were summed and were then normalized by the values at the zero time delay. For each condition, the normalized velocity autocorrelation functions were averaged across all trajectories.

### Live Yeast Cell ATP Sensor and pH Sensor Imaging and Quantification

To image the ratiometric ATP sensor QUEEN in Saccharomyces cerevisiae, TIRF Nikon TI Eclipse microscope was used with 100× Phase, Nikon, oil NA=1.4 objective lens (part number=MRD31901) at around 25° C. Both sensors were illuminated with LED light sources at a single z-plane using DAPI filter set (value recorded as 410ex): excitation filter (Excitation wavelength/Bandwidth (FWHM)=401/17 nm) and an emission filter (Emission wavelength/Bandwidth (FWHM)=444/58 nm), GFP filter set (value recorded as 480ex): excitation filter (Excitation wavelength/Bandwidth (FWHM)=470/40 nm) and an emission filter (Emission wavelength/Bandwidth (FWHM)=525/50 nm). The quantification of QUEEN sensor ratio is mainly followed by standard protocol (119). Basically, individual yeast cells were segmented with 200×200 pixels ROI and the average intensity was measured after background subtraction. The QUEEN sensor ratio was calculated as 410ex/480ex, where a reducing ratio indicates a decrease of intracellular ATP level.

To generate a calibration curve with pH sensor pHurion integrated into the yeast cells (LH4820), the McIlvaine buffer was used to prepare extracellular medium with buffer pH ranging from 5-8, mainly by varying the amount of 0.2M disodium phosphate and 0.1M citric acid. Yeast cells were permeabilized by 100 μg/ml digitonin (Sigma-Aldrich Cat. no. D141-100 MG) while incubated with various pH buffers, 20 mM 2-DG, and 10 UM antimycin A. The pH sensor pHurion was imaged as the same as ATP sensor QUEEN and the sensor ratio was calculated as 410cx/480cx for each measured pH. A linear model was fitted to determine the standard curve, so that the intracellular pH could be quantified upon acute glucose starvation.

### Live Yeast Cell RNP Granules and Q-Bodies Imaging and Quantification

Yeast cells expressing P-bodies protein marker (Dcp2-mScarlet), stress granules protein marker (Pab1-mNeoGreen), or Q-bodies protein marker (Hsp42-mScarlet) were immobilized on the concanavalin A precoated 384-well or 96-well glass bottom imaging plate. Yeast cells were imaged on TIRF Nikon TI Eclipse microscope with 100× Phase, Nikon, oil NA=1.4 objective lens (part number=MRD31901) at around 25° C., LED lights were used to illuminate the preselected field of views, and fluorescence intensities were recorded using GFP and RFP filter set. Acute glucose starvation mediums with/without cycloheximide were switched before imaging. Time lapse movies were set up to record every 10 min for a total of 120 min. 6-μm Z-stacks of yeast cells were taken with 0.5-μm steps between frames. The Z-stacks were projected using average intensity in Fiji, trackmate was used to identify P-body condensates or Q-bodies in individual cells, and the number of P-bodies or Q-bodies were tracked along the time series.

### Live Yeast Cell Total Cell Volume, Vacuole Volume and Cytosolic Volume Measurement

Yeast strain (LH4406) was cultured to log phase and immobilized on concanavalin A precoated 96-well glass bottom imaging plate for 10 min. The remaining cell culture was removed and cells were either washed by synthetic complete medium or acute glucose starvation medium for three times in the imaging chamber, and cells were kept in the respective medium for additional 30 min before imaging. 6-μm Z-stacks of yeast cells were taken with 0.2-μm steps between frames on Nikon TI Eclipse spinning disc confocal microscope. In order to quantify the total cell volume or vacuole volume, the Z-stack images were visually scanned in ImageJ to determine and record center point coordinates for each individual cell or vacuole, based on the respective plasma membrane marker (NeoGreen-PLCΔ-PH2) and vacuole membrane marker (Vph1-TdTomato). The adopted Matlab script was used to identify and segment individual cells and vacuoles through the z-slice, reconstituting them as ellipses for volume quantification (120). The cytosolic volume was calculated by subtracting the vacuole volume from total cell volume in individual yeast cells.

### Immunofluorescence

U2OS cells were seeded in the 24-well glass bottom plate (Cellvis, Cat. no. P24-1.5H-Nor) to reach around 60-70% confluency the next day. After 30 min treatment with the indicated protein translation inhibitors, cells were immediately fixed with flesh 4% paraformaldehyde (Electron Microscopy Sciences, Cat. No. 15714) for 10 minutes at room temperature. The cells were subsequently washed three times with 1×PBS, and permeabilized with 0.2% Triton X-100 (Fisher Scientific, Cat. No. 9002-93-1) in 1×PBS for 15 min at room temperature. Then cells were blocked with 1% BSA, in PBST (PBS+0.1% Tween 20) for 1-hour before applying the primary antibodies (1:250 dilution was applied for anti-G3BP1 antibodies) for overnight incubation in 4° C. The next day, solutions were removed and the cells were washed four times with 1×PBS, and incubated with secondary antibodies (1:1000 dilution was applied for anti-Mouse IgG (H+L)) in 1% BSA, in PBST at room temperature for 1-hour in the dark. Afterwards, solutions were removed and cells were washed with 1×PBS for four times before staining with 1 μM Hoechst 33342 (Thermo Fisher Scientific 62249) for 15 min at room temperature in the dark and were subsequently stored in 1×PBS at 4° C. in the dark until imaging. The fixed plates of cells were imaged on an Andor Yokogawa CSU-X confocal spinning disc on a Nikon TI Eclipse microscope at room temperature. The fluorescence signals were obtained using DAPI epifluorescence (excitation wavelength/bandwidth: 395/25 and emission wavelength/bandwidth: 460/50), RFP laser (Coherent, filter: ET605/70m) and far red (Coherent, ET700/75m) lasers, and images were captured using a Prime 95B scMOS camera (Photometrics) with a 60×/1.49 numerical aperture objective lens. 8-μm Z-stacks of G3BP1 and Depl fluorescence were taken with 0.5-μm steps between frames. The Z-stacks were projected using maximum intensity in Fiji and P-bodies numbers (Dcp1 foci) were manually counted in each cell.

### Poly-dT FISH Imaging

U2OS cells were seeded in the 24-well glass bottom plate (Cellvis, Cat. no. P24-1.5H-Nor) to reach around 60-70% confluency the next day. After 30 min treatment with 10 nM DMDA PatA, cells were immediately fixed with flesh 4% paraformaldehyde for 10 minutes at room temperature. The cells were subsequently washed three times with 1×PBS, and permeabilized with 0.2% Triton X-100 in 1×PBS in room temperature for 15 min. Then cells were washed two times with 1×PBS and one time with 2×SSC (300 mM Sodium chloride, 30 mM Sodium citrate pH 8). The cells were pre-hybridized with the 200 μl pre-warmed (37° C.) hybridization buffer (Molecular instruments, HCR hybridization buffer) in a 37° C. humidified chamber for 30 min. 25 ng of poly-dT-alexa647 probe was prepared for each sample in the HCR hybridization buffer, and it was applied to each well. The plate was kept in a 37° C. humidified chamber in the dark overnight. The next day, the hybridization buffer was removed and cells were washed twice vigorously with a pre-warmed HCR washing buffer (Molecular instruments), followed by an additional two washes of 1×PBS. 1 μM Hoechst 33342 was applied into each well and incubated in dark for 15 min in 1×PBS to stain the nucleus. Cells were subsequently stored in 1×PBS at 4° C. in the dark until imaging. The fixed plates of cells were imaged on an Andor Yokogawa CSU-X confocal spinning disc on a Nikon TI2 X1 microscope at room temperature. The fluorescence signals were obtained with DAPI (Coherent, filter: ET455/50m) and far red (Coherent, ET700/75m) lasers, and images were captured using a Prime 95B scMOS camera (Photometrics) with a 60×/1.49 numerical aperture objective lens. 10-μm Z-stacks of poly-dT FISH fluorescence were taken with 0.2-μm steps between frames.

### Blue-Light Activation of Optogenetic Artificial RNP Condensates

Two days post transient transfection of pLH1972 or pLH2126 into U2OS 40 nm-GEMs cells in the 6-well glass bottom plate, both CRY2-mCherry and CRY2-PUM.HD-mCherry proteins were expressed as diffusive pattern in the cytoplasm. On the day of the experiment, cells were mounted on a Nikon TI2 X1 spinning disk confocal scanning microscope, equipped with a 60×/1.49 numerical aperture objective lens and incubator to maintain 37° C. and 5% CO₂. Individual cells with CRY2-mCherry/CRY2-PUM.HD-mCherry expression were pre-selected initially, and 40 nm-GEMs movies were recorded as described in the Materials and Methods section herein, which reflects particle diffusivity before light induced artificial pumilio granule formation. To apply blue light to induce artificial pumilio granule formation, GFP laser (Coherent, filter: ET525/36m) with 25% power were illuminated to the field of view every 5 sec for 5 min, and artificial RNP condensates signal were recorded by RFP laser (Coherent, filter: ET605/70m). Afterwards, 40 nm-GEMs movies were recorded again.

### Cryo-Electron Tomography

Saccharomyces cerevisiae (BY4741) were recovered from frozen stocks (stored at −80° C.) and streaked onto YPAD agar plates. Cells were grown for 3 days at 30° C. before single colonies were re-streaked onto fresh plates and grown for 2 days as described herein. Single colonies were picked from these plates to inoculate 20 ml of synthetic media supplemented with 20 mM dextrose (glucose control medium). The liquid cultures were incubated overnight at 30° C. and were shaken at 165 rpm (NCU-Shaker mini, Benchmark) to grow cells to the exponential growth phase. Cells at an OD600 of 0.5-0.6 were centrifuged for 3 minutes at 3000 rpm, resuspended in either 1 ml of glucose control medium or synthetic medium supplemented with 100 mM sorbitol (starvation medium) and washed twice. Each washing step consisted of centrifugation at 4,000 rpm for 3 minutes (Eppendorf 5424R centrifuge) and discarding the supernatant and replacing it with 1 ml of the respective medium. Cells were incubated either in glucose control medium or starvation medium shaking at 165 rpm for 30 minutes before plunge-freezing.

Plunge-freezing was performed using a Leica EM GP2 (Leica Microsystems), set to 22° C. and 99% humidity. 4 μl of the cell suspension were deposited on glow discharged holey Quantifoil silicon dioxide grids (R1/2, Cu 200 mesh grid, Quantifoil Micro Tools), and grids were blotted from the reverse side for 1-2 seconds before being plunged into liquid ethane cooled to the temperature of liquid nitrogen. For the subsequent steps of cryo-focused ion beam (cryo-FIB) milling and cryo-electron tomography (cryo-ET), grids were kept at liquid nitrogen temperature.

Cellular slices (lamellae) with a thickness of approximately 200 nm were generated by cryo-FIB milling (121-123). Grids with vitrified cells were mounted into custom-made AutoGrid cartridges and transferred into an Aquilos cryo-focused ion beam/scanning electron microscope (FIB/SEM dual-beam microscope, Thermo Fisher Scientific) using a 45 degree pre-tilt shuttle. Grids were initially sputter-coated with platinum and coated with organometallic platinum using the gas injection system to render the samples conductive and to reduce curtaining artifacts that can occur during FIB milling. Next, positions for lamella generation were determined and refined for eucentricity using MAPS3 (Thermo Fisher Scientific). Milling was performed with a gallium ion beam at 30 kV at a stage tilt angle of 20 degrees. The SEM was used to monitor progress during the milling process (10 kV, 50 pA). Automated rough-milling was conducted in a stepwise fashion with SerialFIB (124) using currents of 1 nA, 0.5 nA and 0.3 nA to thin down the cells to 5 μm, 3 μm and 1 μm, respectively. Finally, lamellae were manually thinned to the target thickness of 200 nm using a current of 50 pA. To render the lamellae conductive for subsequent cryo-ET, a final platinum sputter coating was applied for 3-5 seconds (1 kV, 10 mA, 10 Pa).

Cryo-electron tomograms were acquired on a Titan Krios microscope operated at 300 kV (Thermo Fisher Scientific) equipped with a field-emission gun, a quantum post-column energy filter (Gatan) and a K3 direct detector (Gatan). Data was collected in low-dose mode using automation scripts in SerialEM (125). Tilt series were acquired from −60° to +60° in 2° increments at a magnification of 26,000× with a calibrated pixel size of 3.425 Å, using a dose-symmetric tilt scheme (126), defocus range of 2-4 μm, and keeping the total accumulated dose below 150 e⁻/A². 14 tilt series were acquired for the glucose control and 26 tilt series for the starvation condition. Prior to tomogram reconstruction, tilt movie frames were corrected for gain reference and beam-induced motion in Warp 1.0.9 (127) alongside contrast transfer function (CTF) estimation. Tomograms were aligned and reconstructed by weighted back-projection in AreTomo (128) at 4× binning, corresponding to a pixel size of 13.7 Å.

For localization of ribosomes, 4 tomograms from the glucose control and 10 from the glucose starvation conditions were selected for further analysis based on high data quality (sufficient signal-to-noise ratio due to optimal specimen thickness). Initial particle picking was carried out with DeePiCt (59) on the 4× binned data. To facilitate particle localization and for visualization, tomograms were preprocessed via amplitude spectrum matching to a tomogram of high contrast, implemented in DeePiCt (59). The 3D CNN for ribosome detection (depth D=2, initial features IF=4) was trained on 29 ground truth tomograms (9 volta phase plate, and 20 defocus only S. pombe and S. cerevisiae tomograms) annotated for ribosomes through a combination of template matching, CNN-based predictions using a pretrained network (59; full_vpp_ribo_model_IF4_D2_BN.pth accessible from github[dot]com/ZauggGroup/DeePiCt; de Teresa, I., Goetz S. K., Mattausch, A., Stojanovska, F., Zimmerli C., Toro-Nahuelpan M., Cheng, D. W. C., Tollervey, F., Pape, C., Beck, M., Diz-Muñoz, A., Kreshuk, A., Mahamid, J. and Zaugg, J., Convolutional networks for supervised mining of molecular patterns within cellular context. Nature Methods, 1-11 (2023)), and completed by manual annotation. Performance evaluation of the network resulted in an area under the precision-recall curve (AUPRC) with a median of 0.46 in the S. cerevisiae defocus glucose control data. The 3D CNN thus detected only around 50% of the ribosomes in the data. Undetected particles were therefore picked manually using e2spt_boxer.py in EMAN2 (129) to complete the ribosome annotations in the data to an estimated 90-95% coverage. The complete particle coordinates were then used to reconstruct unbinned or 2× binned subtomograms and corresponding CTF models in Warp 1.0.9 (127) at box sizes of 192×192×192 voxel or 96×96×96 voxel respectively and a particle diameter of 350 Å. Subtomograms of 22,459 particles from the glucose control and 51,846 particles from the glucose starved cells were reconstructed from the respective tomograms. 3D alignment and classification of the subtomograms was carried out in RELION 4.0.1 (130) using a published S. cerevisiae ribosome map (EMDB 3228 (116), scaled and low-pass filtered to 60 Å) as a reference. Multiple rounds of 3D refinement were run in RELION. For the starvation condition, the multi-particle refinement was further carried out in M (131). The resolution of the averages was calculated based on Fourier Shell Correlation (FSC, cut-off criterion 0.143) between the two independently refined half maps. The resolution estimate was 17.8 Å/px for the 2× binned (6.85 Å/px) average from glucose control tomograms and 10.2 Å/px for the unbinned (3.425 Å/px) average from the starved cells. Averages were visualized in ChimeraX 1.1.1 (132).

Based on the ribosome subtomogram averages, the positions of the mRNA entry and exit sites were determined on the maps, and the refined poses for the individual ribosomes used to calculate the distances between the exit site of each ribosome to the entry site of its neighboring ribosome with a custom script in MATLAB 2019a (58). The distance distribution from a ribosomes exit site to the nearest entry site of another ribosome was plotted as a histogram. Gaussians with different cutoff values were fitted to the polysome peak of this distribution, and a distance threshold of 8 nm was found to best describe the data. Consequently, ribosomes within the range of 8 nm of each other were assigned to the same polysome. Finally, each polysome was labeled with a unique identifier and the ribosomes making up the polysome were given sequential numbers. Based on this data, the fraction of ribosomes in polysomes was calculated from the total ribosome counts and the remaining ribosomes were assigned to a monosome fraction. Further, the percentage of ribosomes engaged in different length polysomes was determined by calculating the number of ribosomes assigned to each polysome and determining the frequency of each polysome length. To compare the starvation-induced reduction in polysome fractions determined based on the sucrose gradient fractionation and on the cryo-ET data, polysome fractions were normalized to the respective glucose control fractions. To derive polysome fractions at different distance cutoffs, the above analysis was repeated for mRNA exit to entry site distances ranging from 8 to 25 nm.

### Coarse-Grained Molecular Dynamic (MD) Simulation

A simplified coarse-grained MD simulation framework was developed to represent the most crucial elements of the experimental conditions. The present simulations consist of a mix of spherical particles of diameter 30 nm to represent ribosomes, and varying diameters ranging from 25 to 125 nm representing mesoscale particles. Polysomes can be formed by taking groups of 6 ribosomes in the initial configuration and linking them with harmonic springs of length 40 nm and a spring constant of 20 k_{B}T/nm². In the simulations presented herein, each ribosome in a polysome is linked by a 20 nm spring to a virtual particle diameter 20 nm halfway between ribosomes. This virtual particle only interacts with other virtual particles using a WCA potential described below herein, and serves to prevent polysome chains from crossing, but does not change the excluded volumes for GEMs.

The fraction of ribosomes that are polymerized is varied as a simulation parameter from 0 to 1. A simulation box of size 860 nm per side with periodic boundary conditions was used. The number of ribosomes in the box was set to the integer number that was closest to the target volume fraction of ribosomes. The number of GEM particles was set to the integer number which was closest to 0.5% volume fraction, but was set to 1 for the largest GEMs even if this exceeded 0.5%. RNA and GEM particles had a strictly repulsive interaction of a Weeks-Chandler-Anderson (WCA) form (133).

$${V}_{WCA} ( {r}_{ij} ) = 4 ϵ ( {( {σ}_{ij} / {r}_{ij} )}^{12} - {( {σ}_{ij} / {r}_{ij} )}^{6} ) + ϵ , {r}_{ij} < {2}^{( 1 / 6 )} {σ}_{ij} , otherwise 0 ,$$

with σᵢⱼ being the average of the diameters of the pair of particles being considered, and ϵ=5 k_{B}T.

Additionally, 40 nm spheres representing free RNA were put in the box at concentrations ranging from 0 to 5 micromolar. These particles interacted with each other and with ribosomes through a Lennard-Jones attractive potential (134) with σᵢⱼ set using the same mixing rule, and the potential shifted and cut off at 2.5 σᵢⱼ·ϵ_{I,J}=1.0 between RNA and ribosomes, and between RNA and RNA, but ϵ_{I,J}=0 between RNA and GEMs.

$${V}_{LJ} ( {r}_{ij} ) = 4 ϵ ( {( {σ}_{ij} / {r}_{ij} )}^{12} - {( {σ}_{ij} / {r}_{ij} )}^{6} ) + shift , {r}_{ij} < 2.5 {σ}_{ij} , otherwise 0.$$

MD simulations were performed using the software HOOMD-Blue (135) version 3.7.0. Particle positions were evolved using Brownian dynamics (BD) at a temperature of k_{B}T=1 with a friction coefficient of 0.01, and a time step of 0.0001 simulation units. Simulations were initialized with particles randomly positioned in a box of size 5000 nm per side. Minimization was performed in up to 5 stages of 5000 steps using the FIRE minimization algorithm with a timestep of 0.001, with a force tolerance of 1×10⁻⁴ and energy tolerance of 1×10⁻⁷. Particle velocities were then initialized from a Boltzmann distribution at temperature k_{B}T=1. BD was performed for 10⁵ steps to relax the system, and then the box was compressed to the target volume over 10⁵ steps while simultaneously performing BD. Finally, a production run BD of 5×10⁶ steps was performed and analyzed, saving every 10000 steps, corresponding to one time unit. Most probable displacements were computed by taking the location of the peak in a histogram of root-mean-squared displacements for all GEM particles computed every 100 snapshots. Images of MD simulations were rendered using OVITO (136).

### Example 1. Expression of 50 nm-GEMs in Mammalian Cells

After successfully expressing 50 nm-GEMs in yeast Saccharomyces cerevisiae, this tool was expanded to mammalian cells. The 50 nm-GEMs cassette from the yeast expression vector was subcloned onto a mammalian expression vector by Gibson assembly. HeLa cells were then transiently transfected with 0.5 μg of plasmid DNA, and imaging of the cells was performed 24 hours later on a confocal microscope. HeLa cells expressing green fluorescence all throughout the cell were observed, with seemingly small unassembled particles diffusing throughout the cell. The presence of high fluorescence background throughout the cell made it challenging to do any single particle tracking, and also indicated the presence of a nucleation barrier that prevented the GEM particles from forming. This barrier remained when transfecting cells with a higher concentration of DNA, and also when attempting osmotic shock with sorbitol treatment to facilitate closer proximity of GEM monomers. To overcome this, a short peptide from the intrinsically disordered protein, PopZ, derived from Caulobacter crescentus, was implemented (Lasker K., et. al. The material properties of a bacterial-derived biomolecular condensate tune biological function in natural and synthetic systems. Nat Commun. 2022; 13 (1): 5643. PMID: 36163138). The C-terminal helical oligomerization domain of PopZ was specifically used, which has been shown to be essential for driving phase separation of the PopZ protein. The oligomerization domain was added to the N-terminus of the 50 nm-GEM coding sequence, mediated by a GS linker, via Gibson assembly. With this modified plasmid, HeLa cells were transiently transfected with 0.5 ug of DNA, and improved GEM nanoparticle assembly in all cells was observed. These particles were then able to be analyzed via single particle tracking. As observed in the effective diffusion data from yeast cells, 50 nm-GEMs in mammalian cells were shown to move significantly slower than 40 nm-GEMS, owing to their larger size. This data demonstrated the robustness of GEM nanoparticles, making them a suitable tool to use in different live systems.

### Example 2. CryoEM Structural Analysis

To understand the size and nature of the 50 nm-GEM particles, native purification and structural characterization by cryo-EM single particle analysis (SPA) were performed. Both 40 nm- and 50 nm-GEMS were directly isolated to high purity through anti-GFP affinity purification, though these were not amenable to further structural analysis; thus the 50 nm-GEM was modified to encode a FLAG tag. 50 nm-FLAG-GEMs behaved identically to untagged 50 nm-GEMs with comparable purity after immunoaffinity chromatography. Biophysical characterization by glycerol gradient sedimentation and negative stain TEM indicated immunoaffinity purified 50 nm-GEMs are monodisperse and fully assembled and sufficiently pure for cryo-EM SPA.

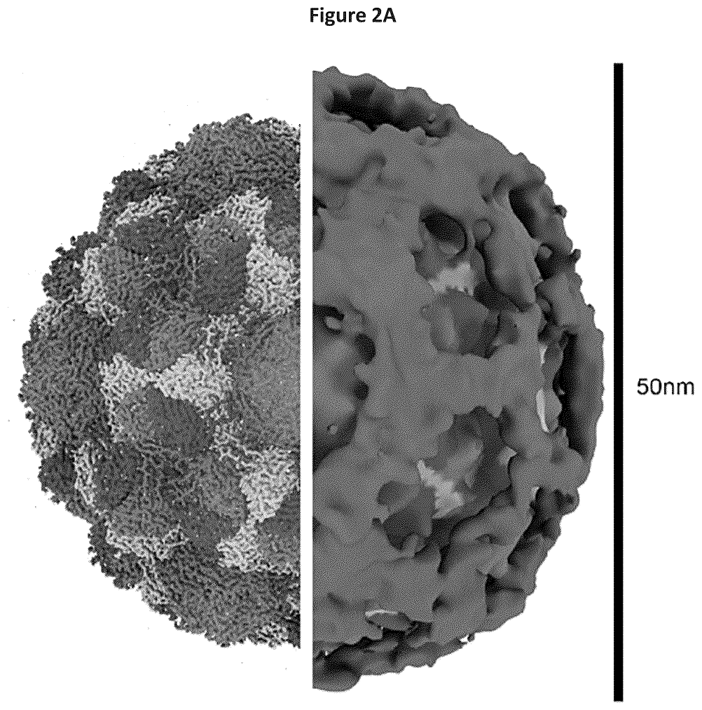

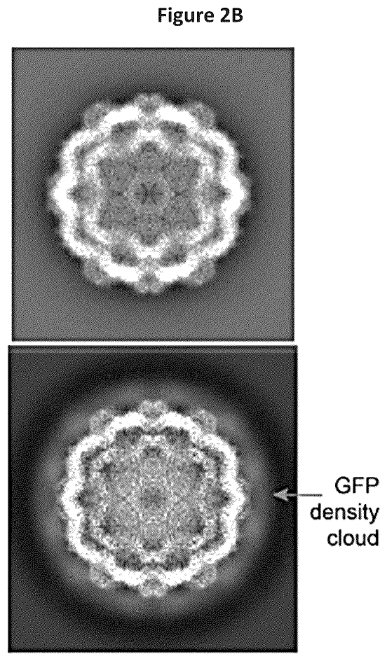

SPA of endogenously purified GEMs yielded a 2.85-angstrom reconstruction, similar to recombinant Quasibacillus thermotolerans two-component encapsulin-based iron storage compartment (Giessen T W, et al. Large protein organelles form a new iron sequestration system with high storage capacity. Elife. 2019 Jul. 8; 8: e46070. PMC6668986). The endogenously purified GEMs formed a ˜42 nm internal scaffold with icosahedral symmetry and T=4 topology (FIG. 2A). The four-protomer asymmetric unit from the endogenous GEM and the recombinant Quasibacillus thermotolerans encapsulin were similar with an all atom root mean square deviation (RMSD) of 0.652 angstroms.

The high-resolution GEM reconstruction, while providing exquisite structural detail, was tragically bereft of significant density for the attached GFP proteins, likely due to their significant flexibility relative to the core scaffold. To obtain information about the full hydrodynamic radius of the 50 nm-GEM, the refinement was repeated with symmetry relaxation, which yielded a 4-angstrom reconstruction with obvious density ascribable to GFP (FIG. 2A). GFP tags formed an apparent cloud around the encapsulin core scaffold with an approximate diameter of 51 nm (FIG. 2A). Comparing projections with the previously studied Quasibacillus thermotolerans encapsulin easily illustrated the presence of the GFP cloud (FIG. 2B). Additionally, cross-sectional analysis of the two maps showed the 50 nm-GEM particle—while having an external cloud of GFP—lacked density previously ascribed to the cargo protein of the Quasibacillus thermotolerans iron storage compartment. This map demonstrated that endogenously expressed GEMs were primarily assembled particles with a particle radius consistent with 50 nm.

### Example 3. Single Particle Tracking Analysis Pipeline

GEM nanoparticles were characterized in vivo by single particle tracking, which revealed similarities in diffusion metrics, but an overall lower diffusion coefficient for the 50 nm-GEM in comparison to the 40 nm-GEM. Here, a single particle tracking analysis pipeline was proposed to gather holistic information about the behavior of GEM nanoparticles in vivo.

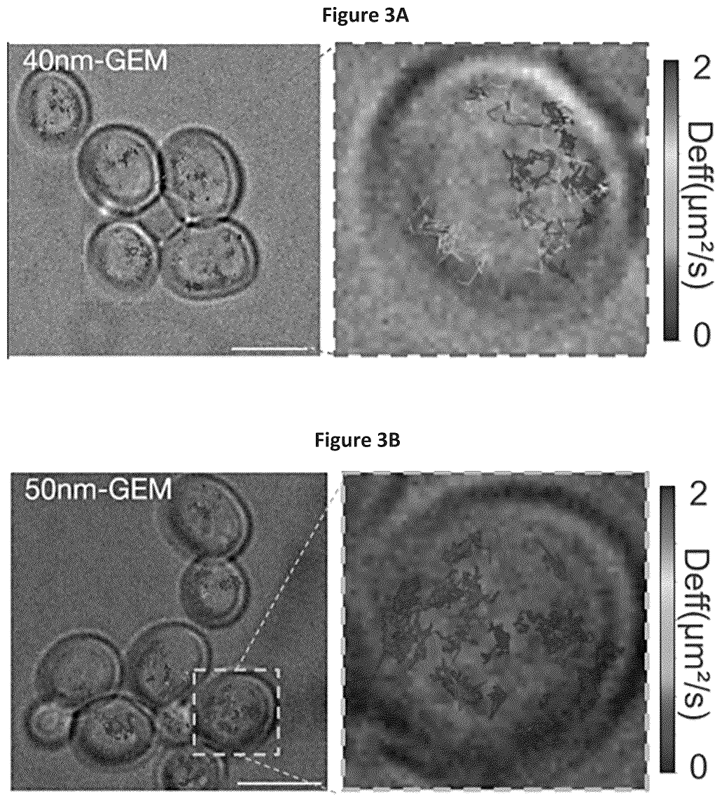

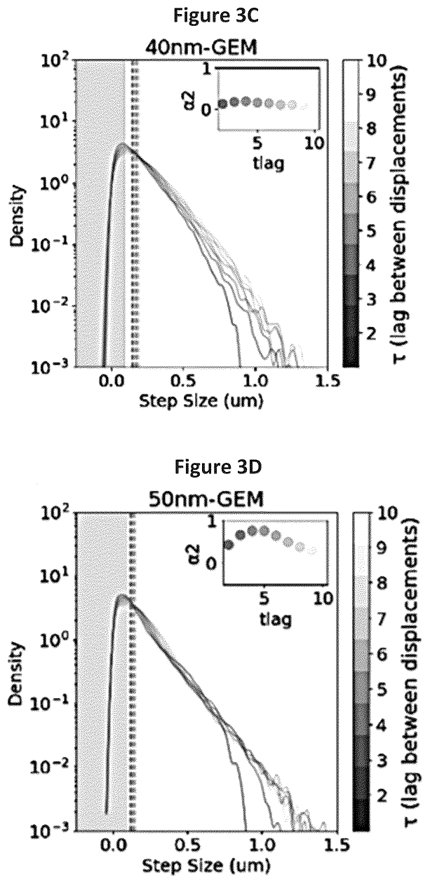

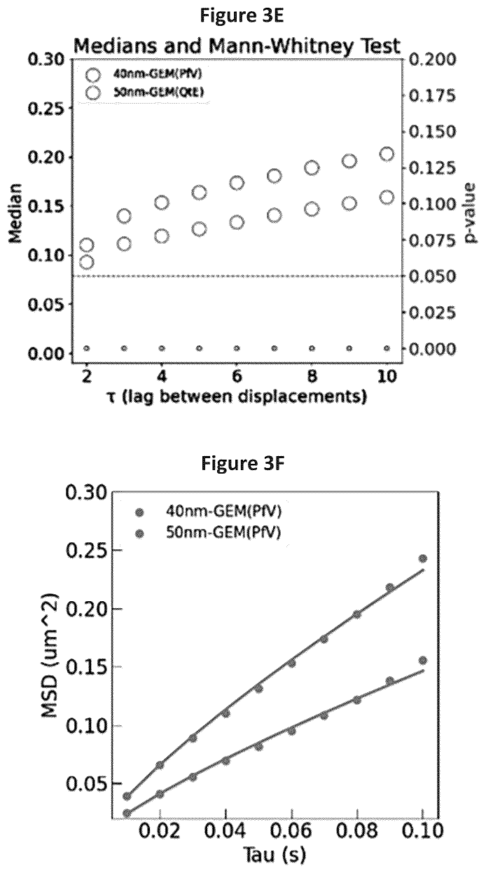

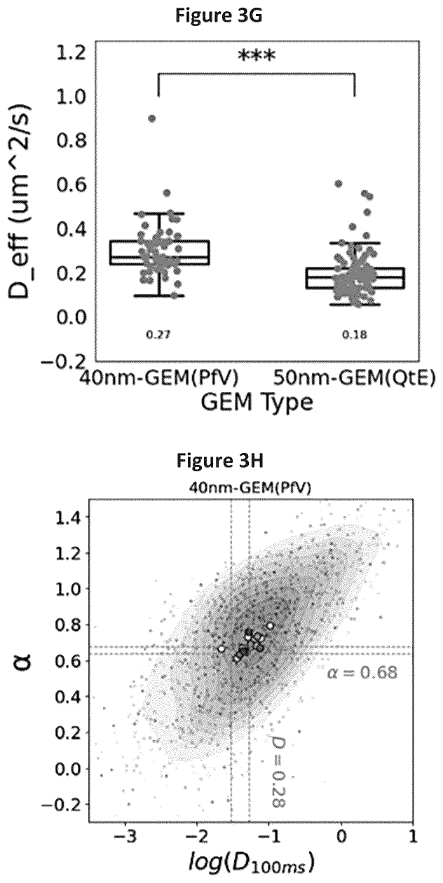

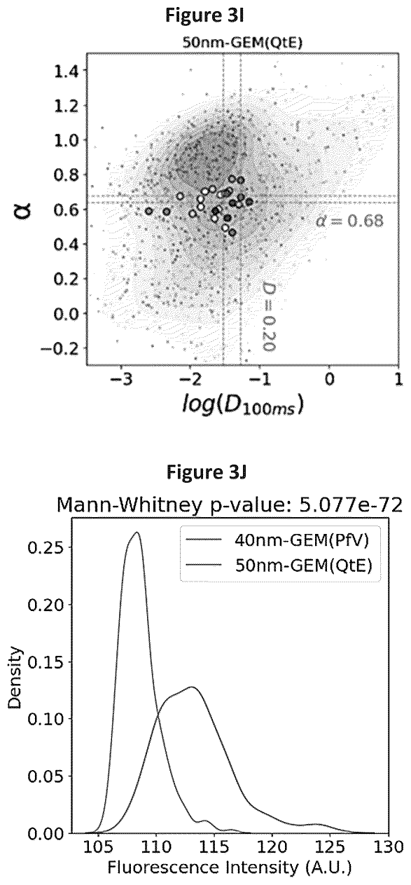

In FIG. 3G, the median effective diffusion coefficient for each cell type was illustrated. A Mann-Whitney (Wilcoxon Rank Test) was conducted to compare the medians (FIG. 3E). The present analysis revealed a statistically significant difference, with the Effective Diffusion Coefficient (EDC) for 50 nm-GEMs being significantly lower compared to 40 nm-GEMs. This finding was consistent with the 10 nm larger diameter observed in 50 nm-GEMs.

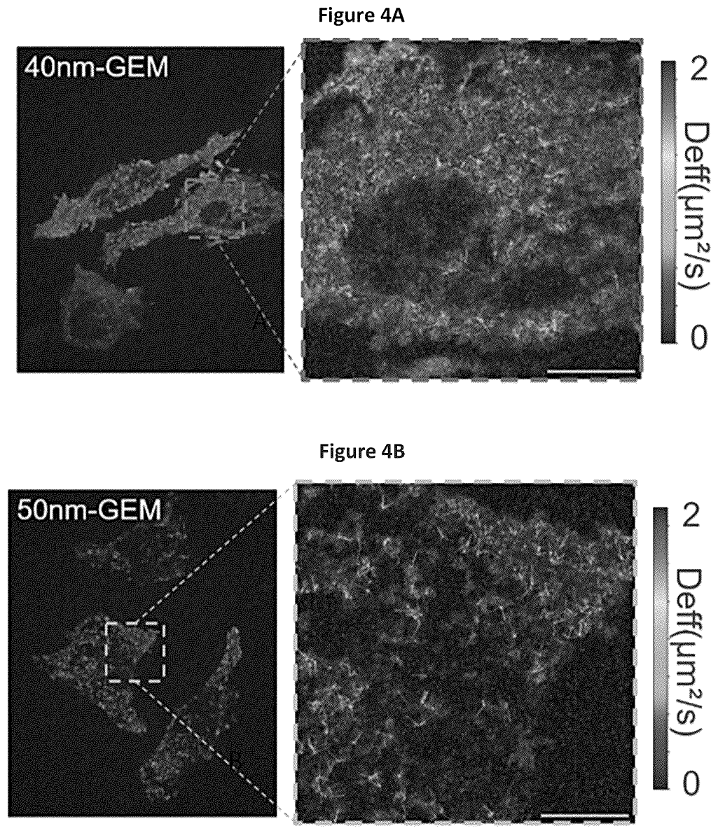

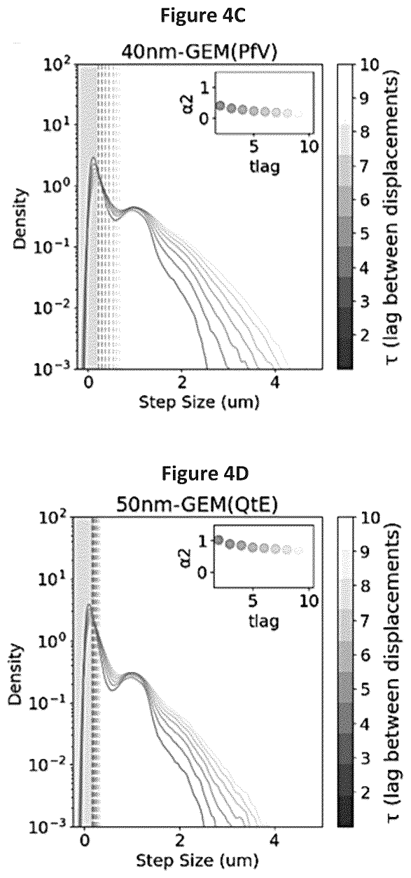

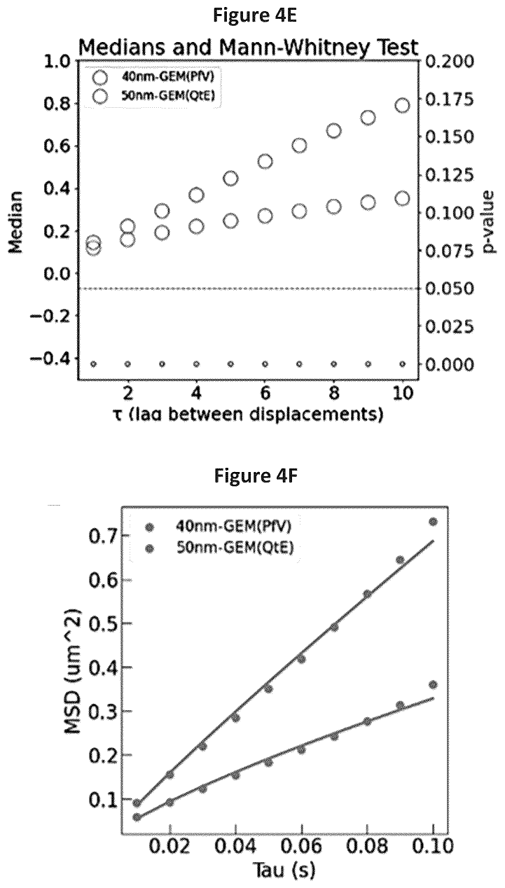

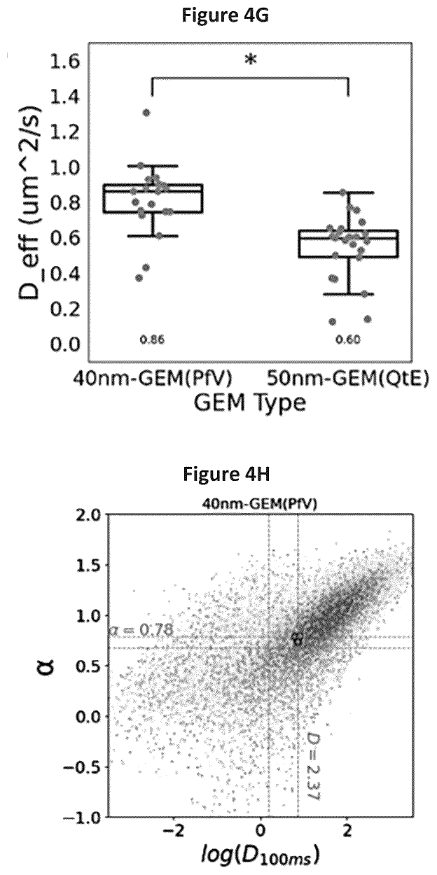

To further investigate the difference in diffusivity reflected by the diffusion coefficients, additional metrics of random motion were examined. Four second GEM movies were taken at a frequency of 100 Hz (period=10 ms), and 400 frames. Single-particle trajectories among the 50 nm-GEMs and 40 nm-GEMs (FIG. 3, FIG. 4) were compared. The particle trajectories were captured using Mosaic in FIJI. For each particle the x,y positions over time were recorded. By measuring the projections of trajectories over time, the amount of area covered by the particles during their diffusion was observed. The representative trajectories plot indicated that typical 50 nm-GEM trajectories explored less space than 40 nm-GEMs (FIGS. 3A-3B, FIGS. 4A-4B).

The step size distribution is the probability distribution of all of the trajectory displacements. Each trajectory has a set of displacements calculated as the difference between consecutive positions. The ensemble of all trajectory displacements is then binned, and the step size distribution calculated. The step size distribution revealed lower medians for 50 nm-GEMs compared to 40 nm-GEMs. Moreover, the alpha2 parameter consistently approached zero across all t_lags, indicating Gaussian distributions. These distributions demonstrated consistent behavior across different time lags (tau), with 50 nm-GEMs displaying more uniform distributions than 40 nm-GEMs. Neither type of GEM exhibited heavy-tailed distributions, as evidenced by the lack of sharp decreases in step size distributions. Additionally, the median step size distribution of 50 nm-GEMs were consistently lower than those of 40 nm-GEMs. Notably, there was no indication of barriers impeding GEM motion, which would manifest as sharp decreases in all step size distributions.

The parameters \(\alpha \) and \(D\) were fit using the anomalous diffusion equation, focusing specifically on the mean squared displacement (MSD). MSD analysis provides critical insights into the dynamics of particle motion by examining trajectories over various time intervals (\(\tau \)). The MSD for each trajectory was calculated to determine the average displacement over time, allowing characterization of different types of particle motion. Different motion types can be identified by distinct patterns in the MSD versus \(\tau \) plots (James A. Dix and A. S. Verkman. Crowding effects on diffusion in solutions and cells. Annual Review of Biophysics, 37 (0): 247-263, 2008). Pure Brownian motion is indicated by a linear relationship between MSD and \(\tau \), while subdiffusive motion is characterized by an \(\alpha \) value less than one. Variations in \(\alpha \) often indicate confined motion or spatial heterogeneity. Most trajectories occur within a duration of 10 to 100 milliseconds. Consequently, the present analysis was focused on \(\tau \) values ranging from \(10\hat{ }{-2} \) to \(10\hat{ }{-1} \) seconds to adequately capture the most common trajectories. By fitting the MSD versus \(\tau \) data to Einstein's Diffusion Equation (A. Einstein. Annalen der physik. 17:549-560, 1905), the Effective Diffusion Coefficient (\(D_{\text {eff}} \)) was derived. This coefficient provided a quantitative measure of the GEM nanoparticle dynamics in vivo.

The analysis was begun by linearizing the anomalous diffusion equation using a logarithmic transformation. This step was followed by fitting a linear regression to the transformed data, which yielded a variety of solutions. These solutions were then utilized to generate trajectories, with each trajectory subsequently fitted with a linear regression line. Solving for \(\alpha \) and \(\log D \), the \(\alpha \) vs. \(\log D \) space was populated with data points corresponding to each trajectory.

The next phase involved using Kernel Density Estimation (KDE) to group these data points. The technical details of equation solving and KDE implementation were disclosed in the Materials and Methods section herein. Rather than using centroids, the resulting medians of the distributions were plotted. This approach was particularly useful during the single-cell analysis, as it provided a clearer depiction of changes in diffusivity.

The diffusion form was estimated from these plots. For Brownian diffusion, a distribution centered around \(\alpha=1 \) was expected, with the KDE plot revealing the spread around this central value. This analysis set the stage for characterizing various motion types, such as macromolecular crowding, which deviates from Brownian motion and displays distinctive patterns. For example, confined motion is often associated with a lower diffusion coefficient.

The motion of each trajectory was analyzed by fitting the MSD vs. tau plot to a line. The parameters of this line correspond to the alpha value (slope) and diffusion coefficient (intercept).

The one dimensional Einstein equation was: [symbol] δx² [symbol] =2D_{eff}t, which for two dimensions was:

$$〈 δ {r ( x , y )}^{2} 〉 = 4 {D}_{ff} t .$$

Subdiffusive behavior within the cell environment was anticipated due to the presence of diverse cytoplasmic components. A slope lower than one in this context is indicative of subdiffusion (James A. Dix and A. S. Verkman. Crowding effects on diffusion in solutions and cells. Annual Review of Biophysics, 37 (0): 247-263, 2008). Moving forward, the effective diffusion coefficient, Deff, was examined which offers insights into particle movement speed. Deff was derived from solving the anomalous diffusion equation [symbol] Δr\hat{ }2 [symbol] =4Dtα, with an assumed a value of 1, simplifying to [symbol] Δr\hat{ }2 [symbol] =4Deff. The present analysis revealed a reduction in Deff for 50 nm-GEMs compared to 40 nm-GEMs. This decline was likely influenced by the size discrepancy between the two types of GEMs, as larger particles tend to experience slower diffusion rates due to increased friction and hindered movement within the medium.

The alpha versus logD plot was examined in order to delve deeper into this phenomenon. This analysis allowed exploration of both parameters of the anomalous diffusion equation without assuming a specific type of motion. Each trajectory's anomalous diffusion parameters were fitted and then plotted in a two-dimensional space as small dots, while the median values per cell were represented by larger dots.

The diffusion coefficient and alpha value for both types of GEMs were calculated and visualized in this parameter space. Trajectories tended to cluster around alpha values close to one, with a median of 0.72. However, the confidence interval for the alpha value included one, as revealed by bootstrapping (n=1000). For 50 nm-GEMs, particles predominantly concentrated around an alpha value of one, whereas 40 nm-GEMs exhibited more concentration around a specific value with less dispersion along the x-axis.

Distinct group formations were observed in 40 nm-GEMs, indicating deviations from the typical Brownian diffusion pattern. A downward shift within a group implied anomalous diffusion with equivalent diffusion coefficients but lower alphas, while a leftward shift suggested reduced diffusion coefficients while maintaining an alpha value of one. Notably, the distributions around the kernels in 40 nm-GEMs remained sufficiently close to one without forming a bimodal distribution.

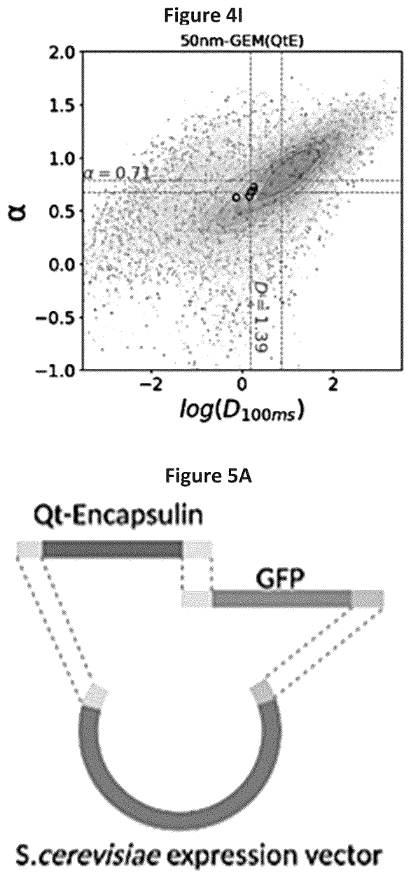

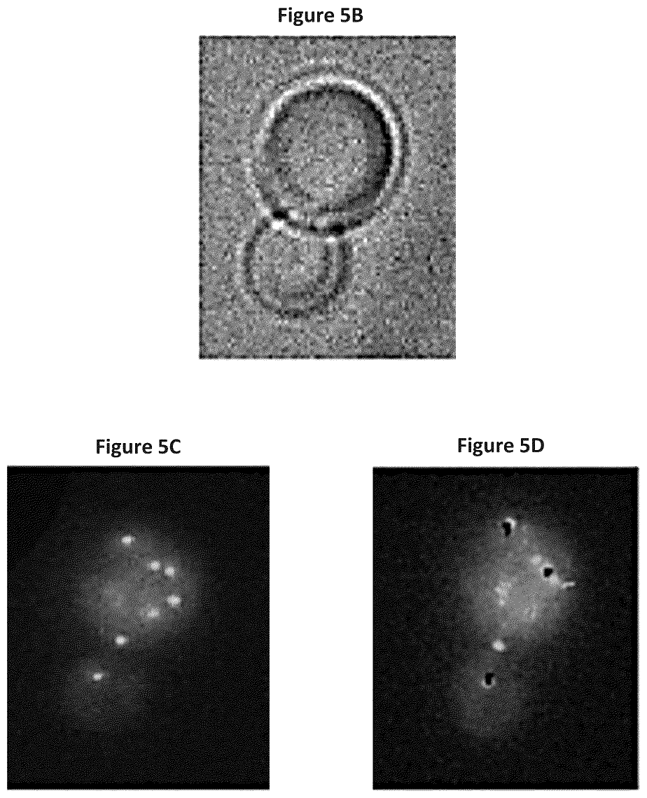

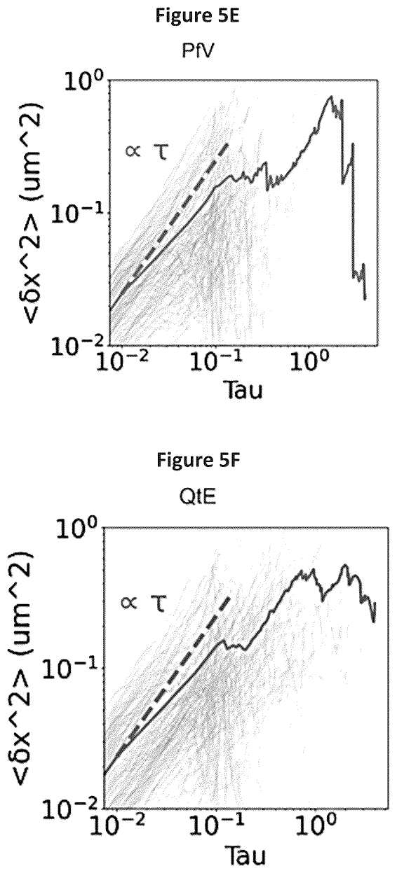

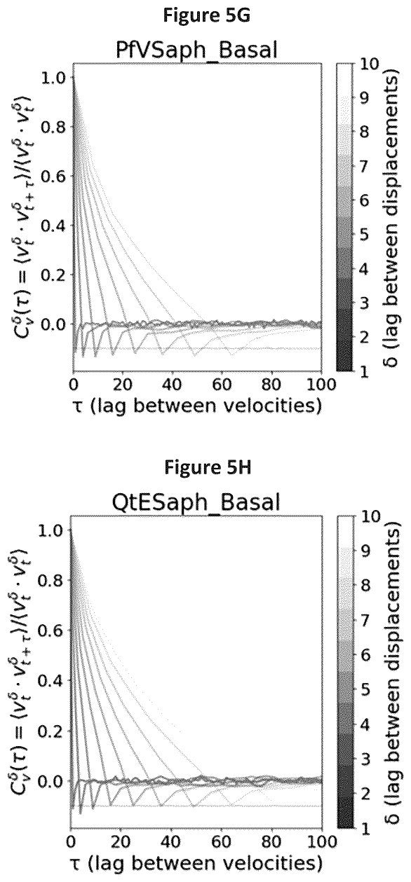

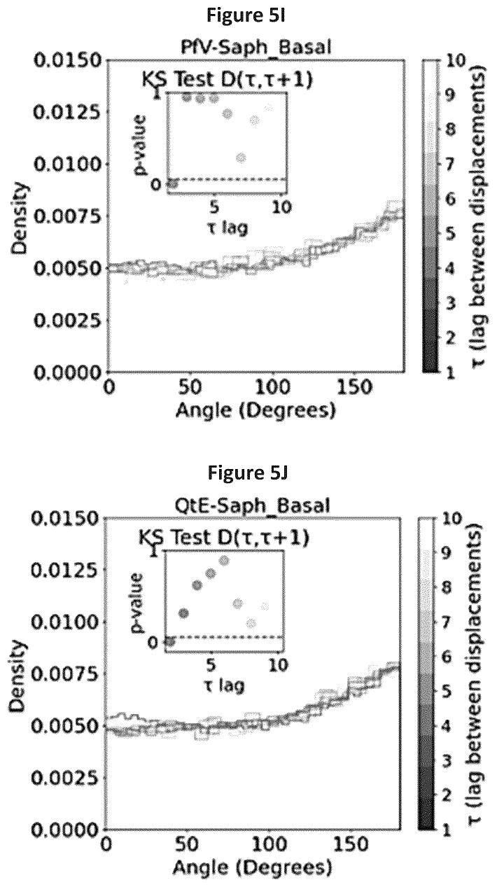

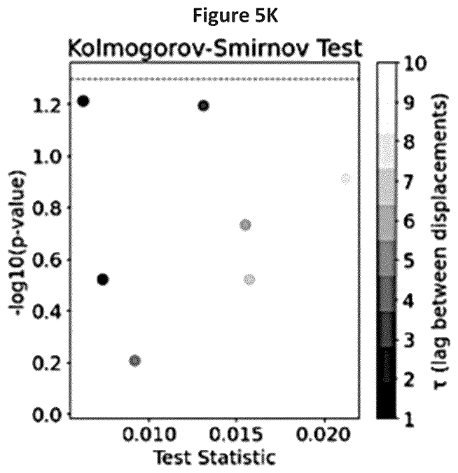

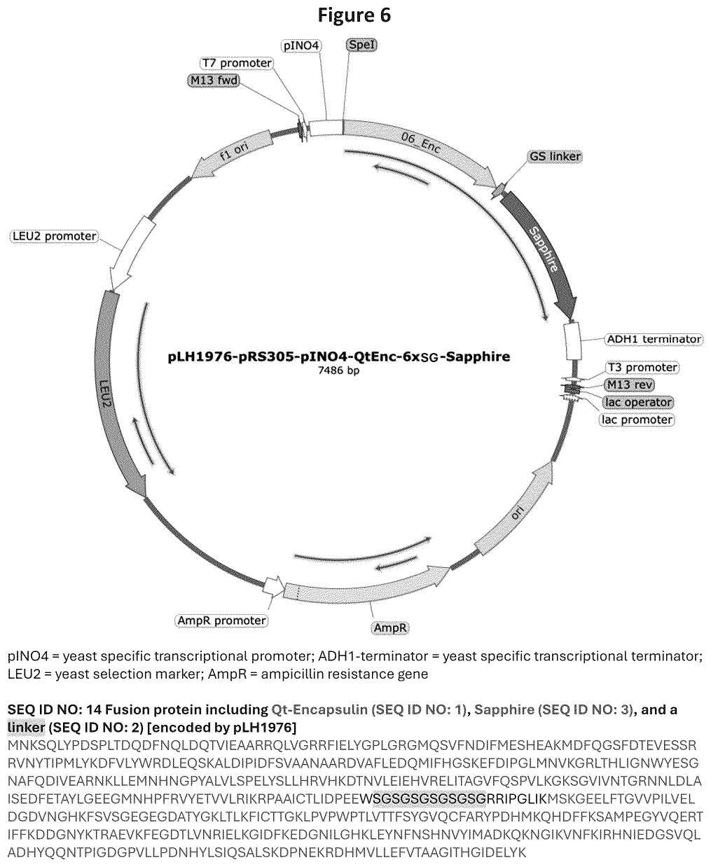

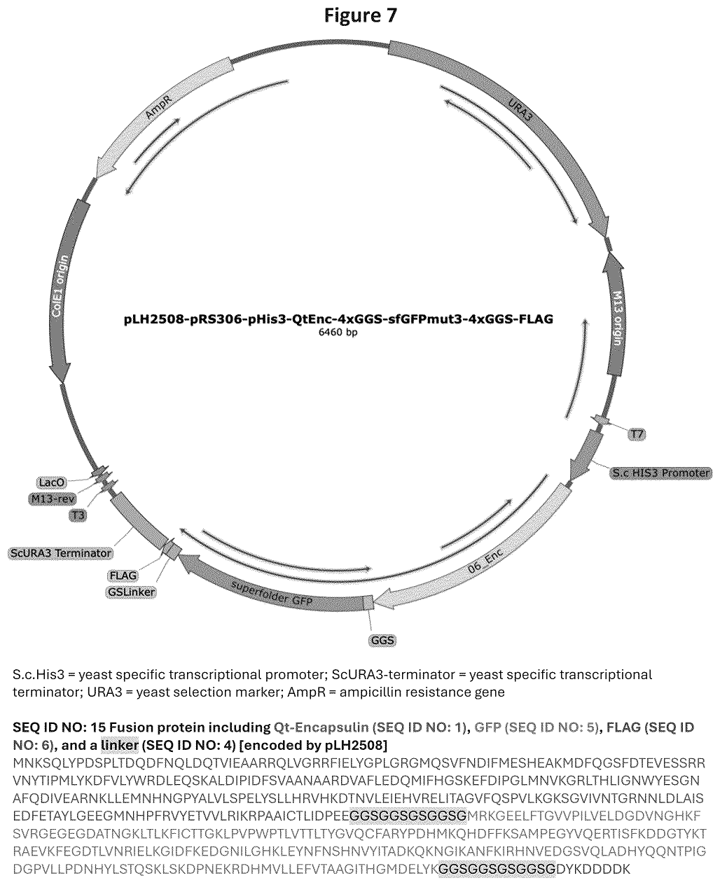

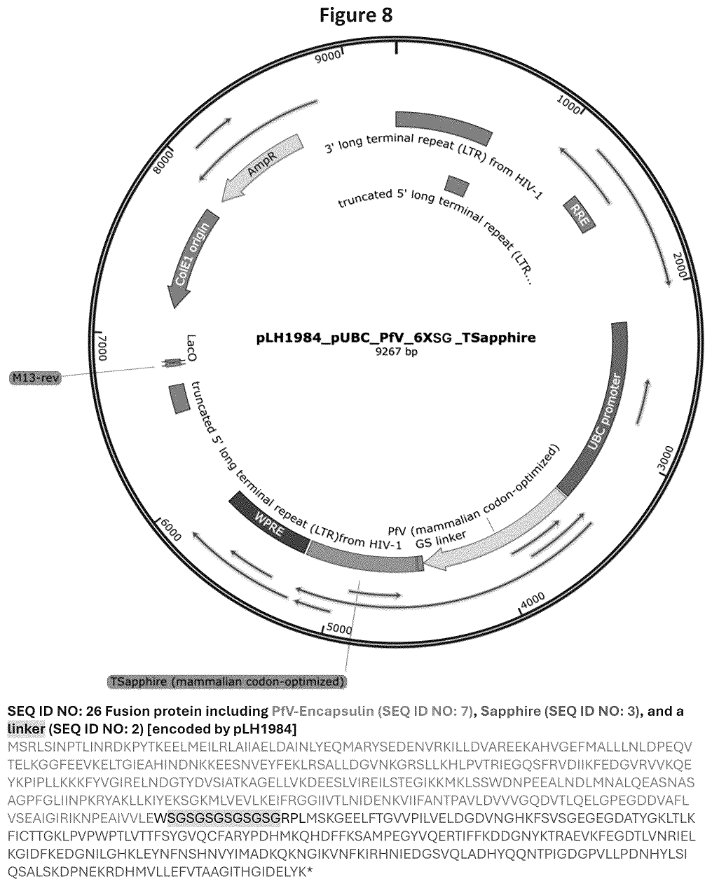

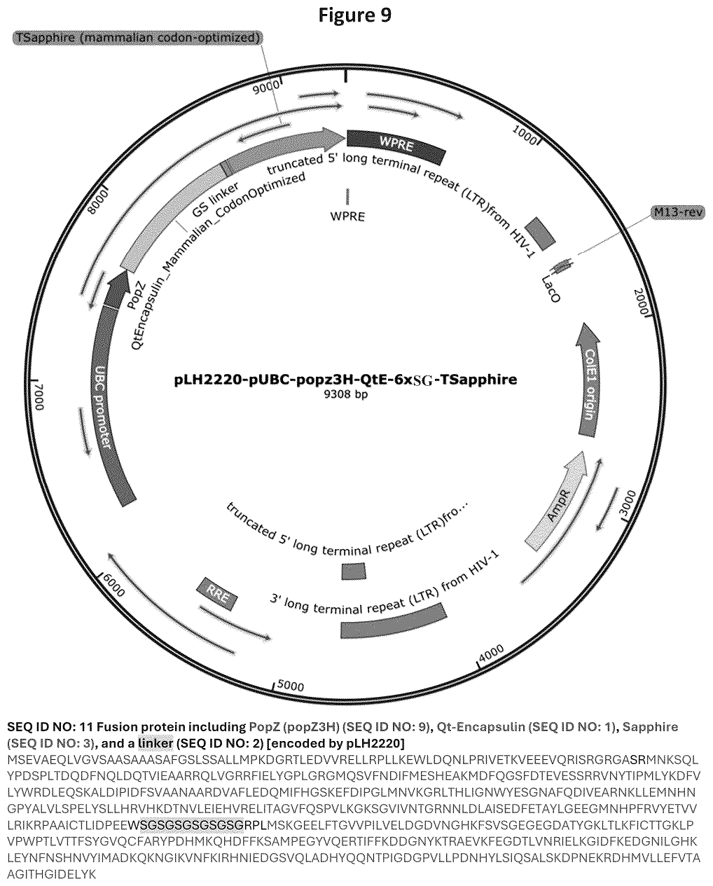

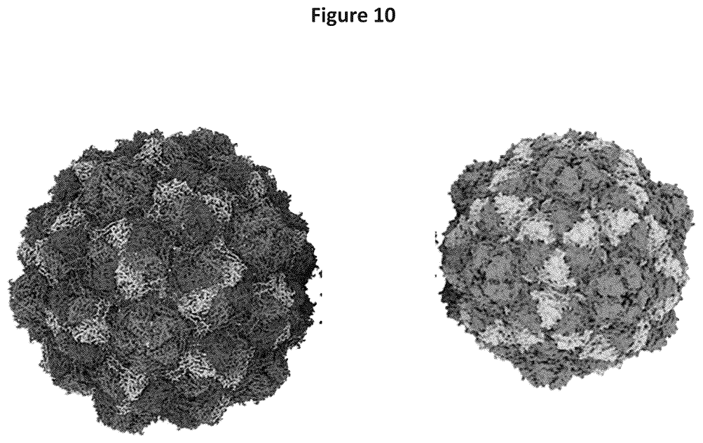

The velocity of a correlation function was then calculated. The Velocity Auto Correlation Function showed the case to predict the next velocity step based on the previous step. A VACF=1 means that it is 100% likely to predict the next velocity step. A VACF=0 means that the next step cannot be predicted. A VACF=−1 means that the next velocity step, although predictable, goes in the complete opposite direction. This means that if there are more values on the negative side, there is more likely to be confinement of the particles. If the VACF goes from 1 directly to zero, that means the trajectory is Diffusion with Brownian Motion. The Velocity Auto Correlation Function showed an almost constant valley with delta. A sub diffusive signature was observed. As shown in FIG. 5 the peak valleys of the distribution of the velocity of a correlation function were close to constant for each delta.

Regarding the angle distribution observed in both 40 nm- and 50 nm-GEMs, in PfV GEMs, there was a small peak near 180 degrees, indicative of some degree of confinement, though not total. Interestingly, this peak was also observed in 50 nm-GEMs, suggesting a similar level of confinement. However, it was important to note that this confinement did not result in any observable restrictions, as evidenced by the MSD tau and alpha D values. Furthermore, the sensor sensitivity also played a crucial role in accurately capturing these distributions.

### Example 4. Mesoscale Diffusivity Transiently Increases Upon Diverse Types of Stresses

Microrheology is the inference of the physical properties of a material from the observation of the motion of passive probes embedded within the environment (35). Passive rheological probes have proven to be effective tools for inferring the biophysical properties of different cellular compartments (1, 3, 21, 36-39). Previous reports have described decreased motion of mesoscale particles upon acute glucose starvation (2). However, these observations monitored diffusivity of mRNP probes, which were later found to strongly interact with P-bodies, preventing the inference of general cytoplasmic properties from changes in their diffusivity (40). Furthermore, using passive rheological probes, increased mesoscale diffusivity upon acute glucose starvation was identified (41). To follow up on this observation, it was sought to characterize the dynamics and mechanisms underlying this change. Therefore, the present analysis was expanded to include additional time points. In addition, physical properties of the cytoplasm at two length-scales were investigated by using both u NS particles (50-150 nm diameter) (1) and 40 nm-GEMs (40 nm diameter) (21).

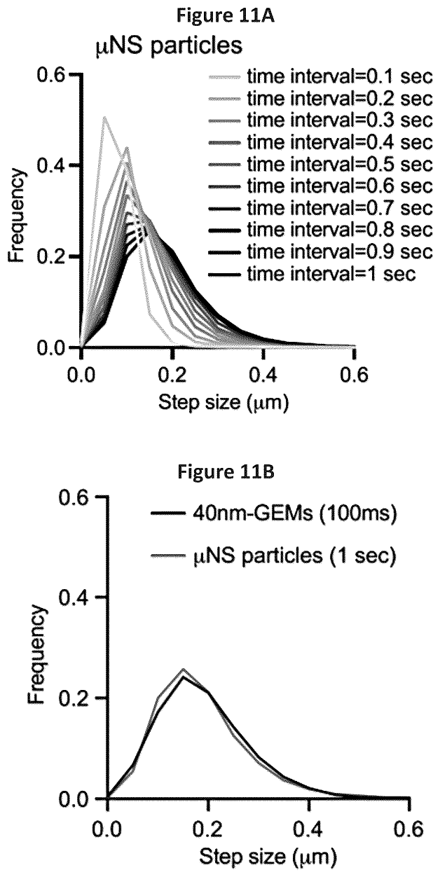

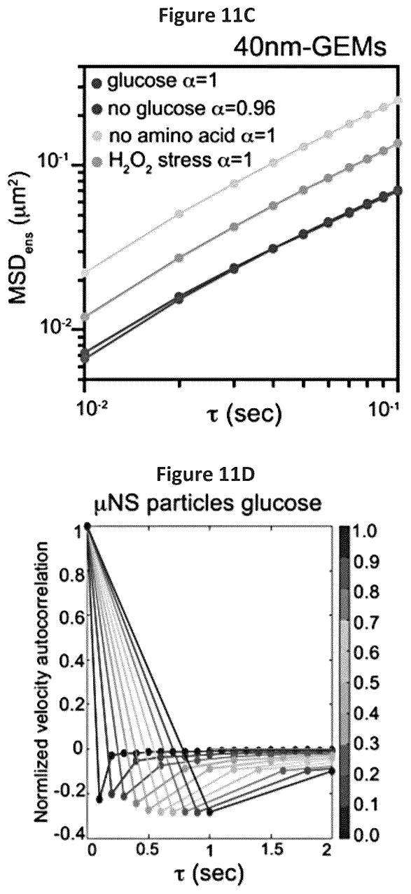

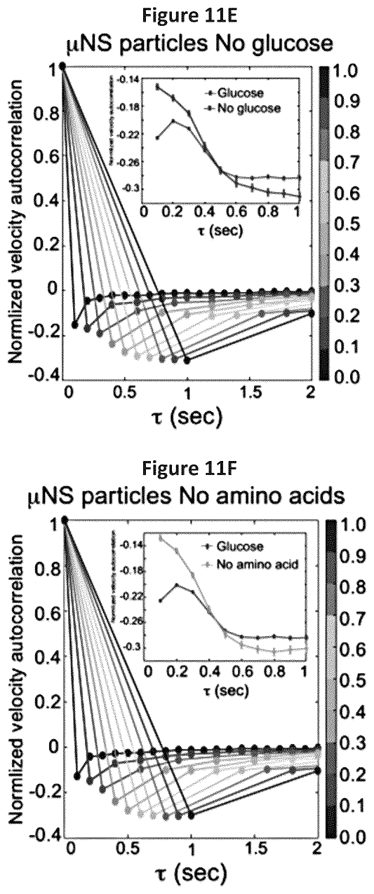

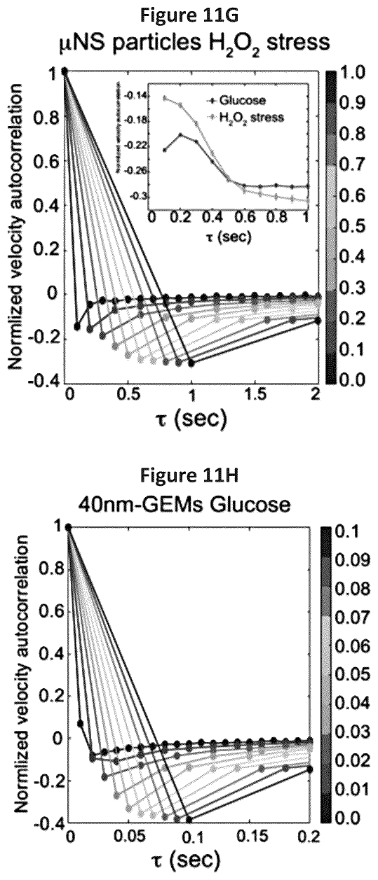

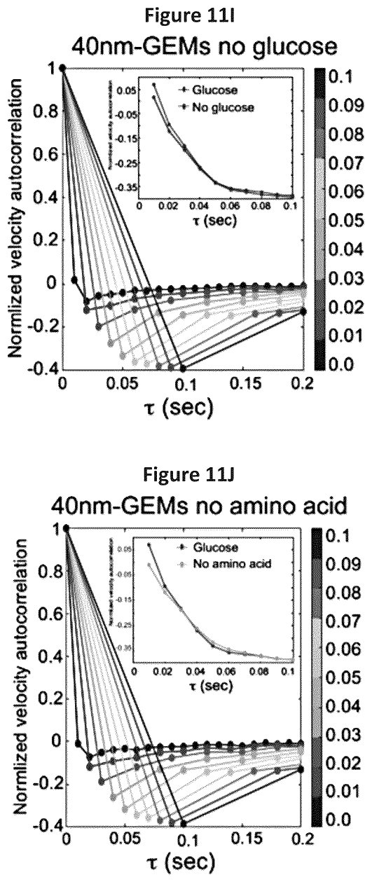

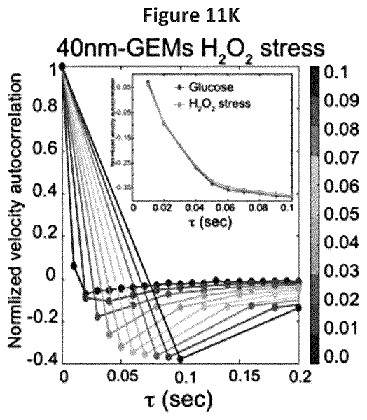

As the cytoplasm does not satisfy the simplifying assumptions of the Stokes-Einstein equation (19,42,43), it was not possible to assign a single diffusion constant for a particle. Rather, the effective diffusivity of particles must be assigned at a specific time-scale. A timescale of 100 ms (D_{eff_100ms}) for 40 nm-GEMs and 1 second (D_{eff_1s}) was chosen for μNS particles because the step-size distribution of these particles was similar to each other at these time intervals (FIGS. 11A-11B).

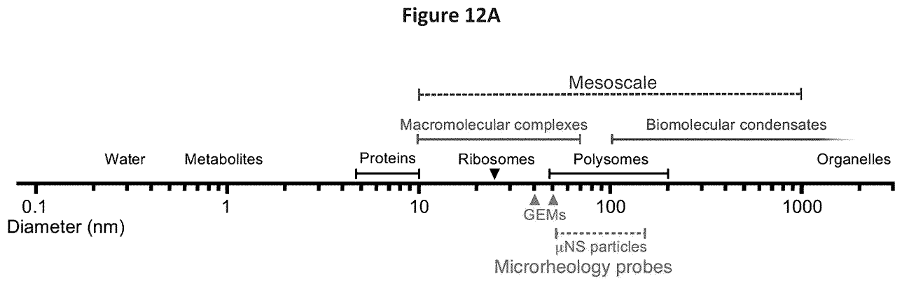

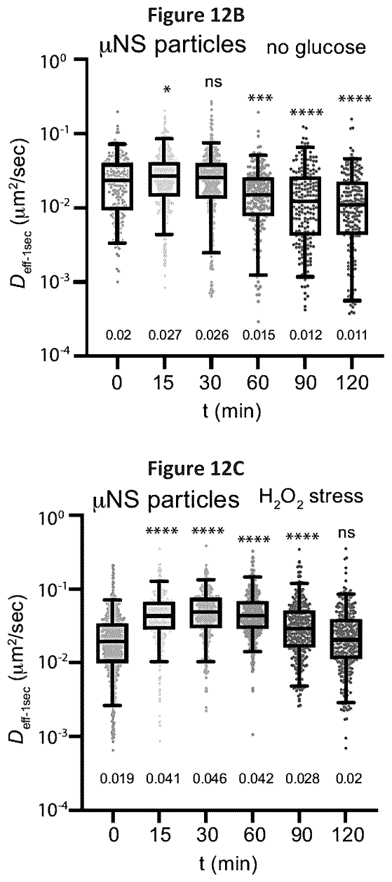

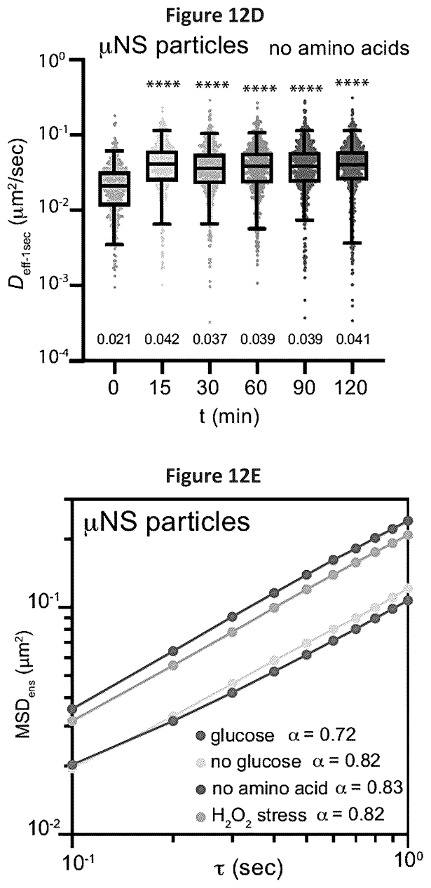

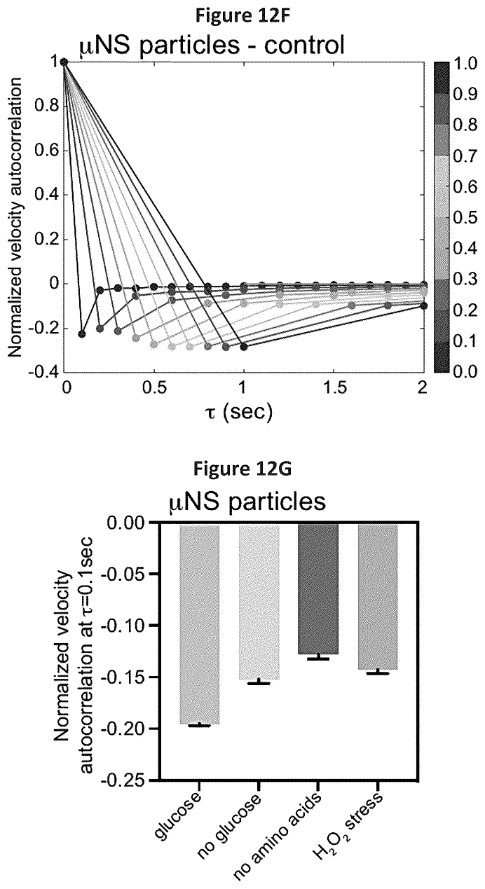

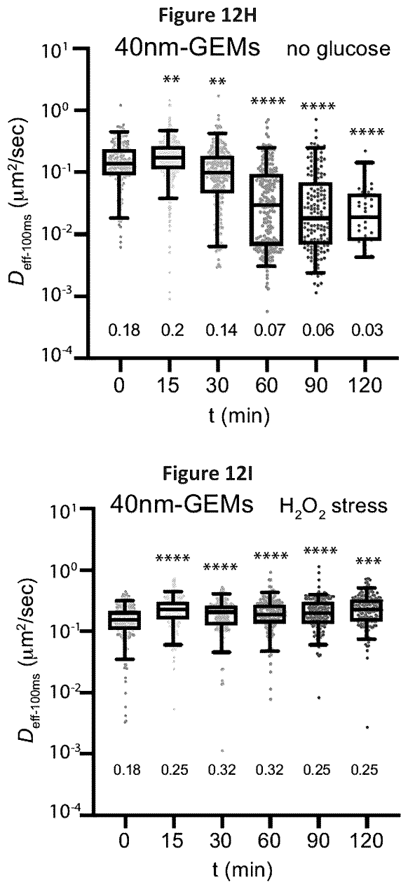

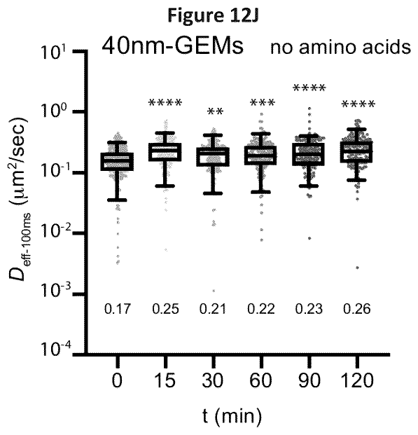

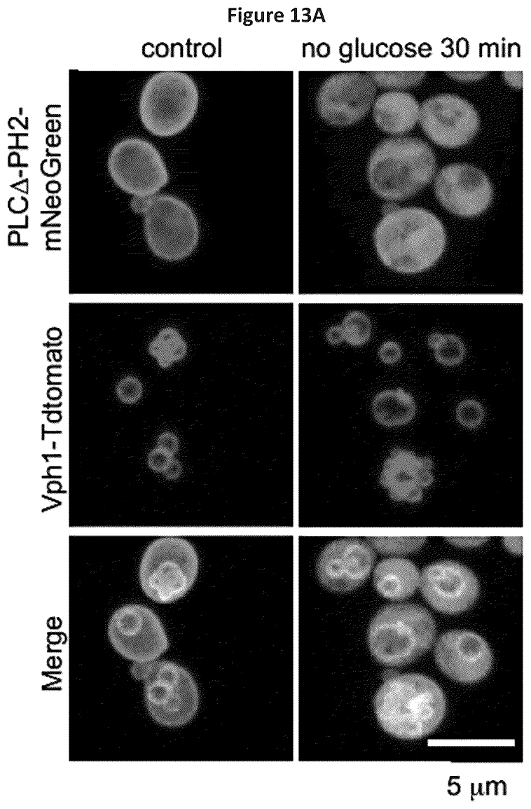

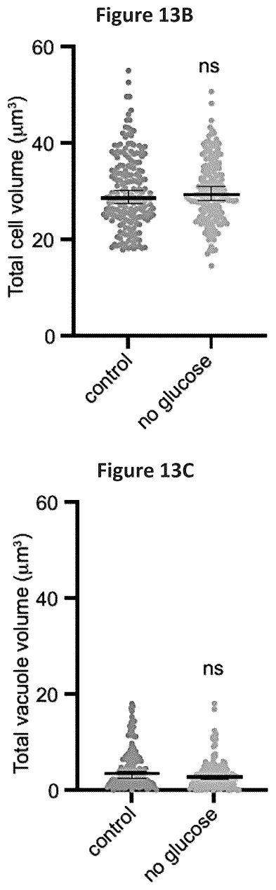

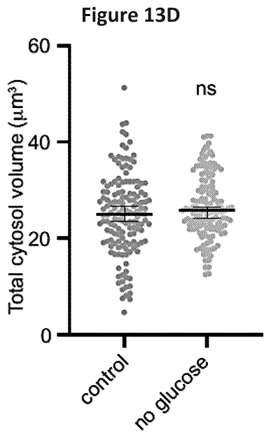

In agreement with (41), a transient increase in the D_{eff_1s} of μNS particles was found immediately after glucose starvation that was most significant at the earliest time point assayed (15 min) and returned to initial values after 1 h (FIG. 12B). Note, all media were carefully osmotically balanced, and it was confirmed that there was no significant change of cytosolic volume upon acute glucose starvation (FIG. 13). Two additional stress conditions-oxidative stress with H₂O₂ and amino acid starvation-were examined, and it was found that both stresses caused increased diffusivity of μNS within 30 min (FIGS. 12C-12D). Interestingly, the subsequent dynamics of mesoscale diffusivity varied between stresses: in glucose starvation, diffusivity ultimately decreased below initial values; after oxidative stress, diffusivity ultimately returned to initial values; after amino acid starvation, diffusivity remained high even after 2 hours (FIGS. 12B-12D).

The anomalous exponent α value of μNS particles was consistent with sub-diffusive motion (α<1, FIG. 12E). Anomalous subdiffusion can arise for multiple reasons. It was found that mean-square-displacement did not reach a plateau over the time scale of the experiment, suggesting the μNS particles were unlikely to be under rigid confinement. Other possible reasons for subdiffusion included interactions with the environment, and elastic confinement. Elastic confinement gives rise to specific forms of time-correlated motion (44). μNS particle trajectories had a negative velocity autocorrelation on short time-scales, consistent with elastic memory (FIG. 12F, FIGS. 11D-11G). The value of this autocorrelation was variable depending on time-scale, again consistent with elastic behavior rather than rigid confinement (FIG. 12F, FIGS. 11D-11G) (44). To simplify visualization, a single tracking time-scale was used to compare multiple conditions (τ=0.1 seconds) (FIG. 12G). In WT cells, it was observed that the negative velocity autocorrelation value was diminished in all three stress conditions, suggesting that clastic confinement was decreased (FIG. 12G).

40 nm-GEMs also displayed a transient increase in D_{eff_100ms} after 15 minutes of glucose starvation, and maintained high mesoscale diffusivity in both amino acid starvation and oxidative stress with H₂O₂ (FIGS. 12H-12J). However, the anomalous exponent α value of 40 nm-GEMs remained around 1 in all tested stresses, suggesting that these smaller particles experienced less clastic confinement (FIG. 11C). Again, the elastic confinement varied depending on time-scale, but obvious differences between control and stress conditions were not found (FIGS. 11H-11K).

### Example 5. ATP Levels and pH Decrease Upon Glucose Starvation

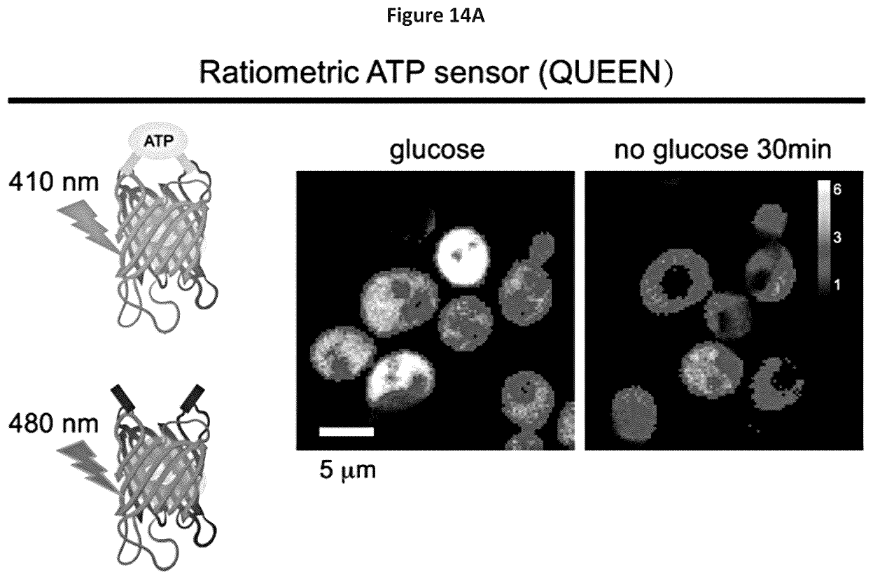

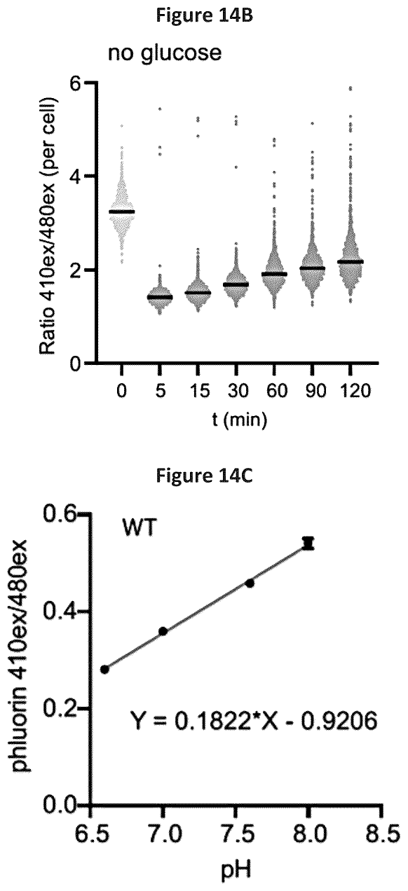

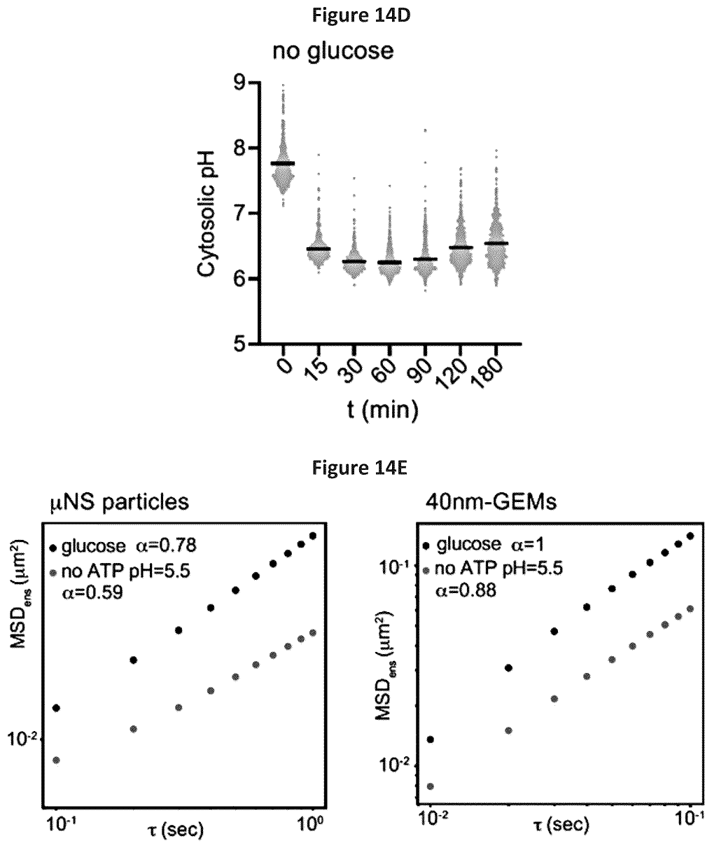

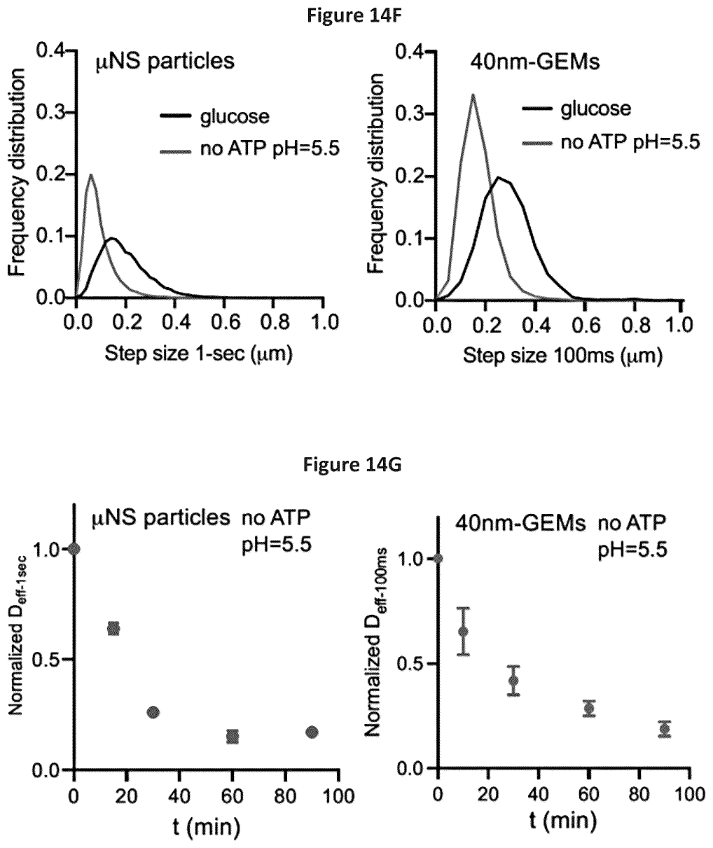

The physical properties of the cytoplasm undergo complex changes over time following glucose withdrawal: first mesoscale diffusivity increases, but later it decreases. Budding yeast are adapted to acidic environments, and the plasma membrane proton transporter PMA1 uses large amounts of ATP to maintain a near neutral intracellular pH (2,45); this pump inactivates during starvation to conserve energy. Consequently, both ATP levels and pH decrease upon glucose withdrawal (2,46). The ratiometric fluorescent ATP sensor, QUEEN (47,48) and the ratiometric pH sensor, pHluorin (49) were used to quantify ATP levels and intracellular pH. It was found that ATP levels plummeted within 10 minutes of acute glucose starvation (FIGS. 14A-14B), while intracellular pH reduced to 6.6 from the log-phase value of 7.7 after 10 minutes of glucose starvation and fell to 6.3 after 30 minutes of glucose starvation, consistent with earlier reports (FIGS. 14C-14D). Previous studies have shown that ATP depletion experiments in media without glucose and supplemented with 2-deoxyglucose (2-DG, an inhibitor of glycolysis) and antimycin A (an inhibitor of mitochondrial respiration) in an acidic environment drastically reduced movement of endogenous particles as well as μNS particles after 2 hours treatment (3). The same drug treatments were performed for ATP depletion experiments in media buffered at pH 5.5 without glucose with additional early timepoints. It was found that both μNS particles and 40 nm-GEMs displayed a reduction in effective diffusivity (a values) and a shift to smaller step sizes after 30 minutes (FIGS. 14E-14G). These results suggested that decreases in pH and ATP drive the ultimate decrease in diffusivity after an hour of glucose starvation. However, the initial increase of mesoscale diffusivity was likely to be due to other intracellular changes. It was sought to identify those factors.

### Example 6. Disassembly of Polysomes is Required for Increased Cytoplasmic Fluidity

In the cytoplasm, ribosome concentration is a major determinant of mesoscale particle diffusivity. Inhibition of TORC1 signaling reduces ribosome concentration and leads to increased diffusivity of mesoscale particles (21). However, this change relies on autophagy and takes more than one hour. Increased diffusivity and decreased elastic confinement were observed after less than 15 minutes of acute stress. Therefore, it was hypothesized that other properties of ribosome physiology could explain the rapid fluidization of the cytoplasm upon acute glucose starvation and other stresses.

In rapidly proliferating yeast cells, more than 80% of ribosomes are engaged in protein translation (50,51) often with multiple ribosomes simultaneously translating one mRNA, resulting in polysome complexes (polysomes) (52-54). Upon acute glucose starvation, various mechanisms rapidly inhibit translation initiation, leading to disruption of polysomes (24). Ribosomes dissociate from mRNA, disassembling into 40S and 60S ribosomal subunits (25) and then rapidly reassemble into 80S monosomes in an STM1-dependent process (55,56). It was hypothesized that collapse of polysomes might transiently fluidize the cytoplasm in stress conditions.

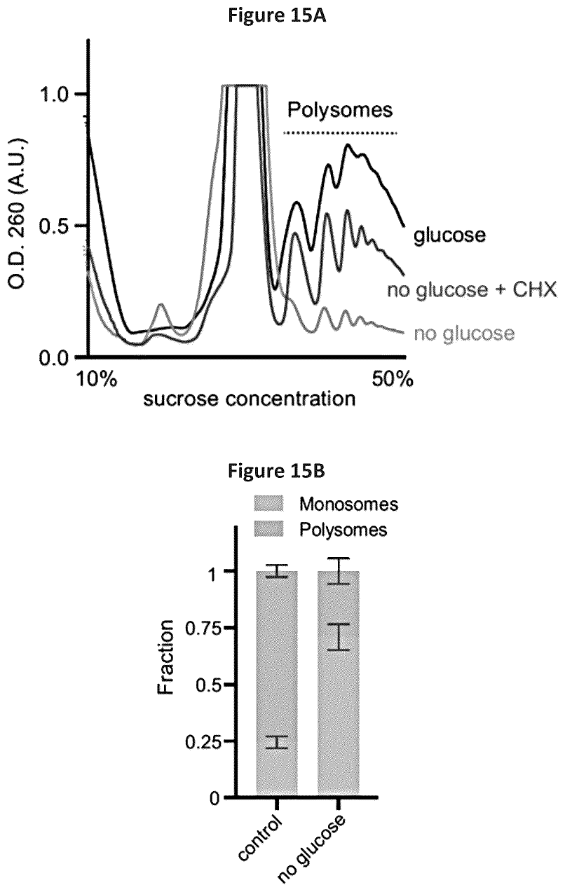

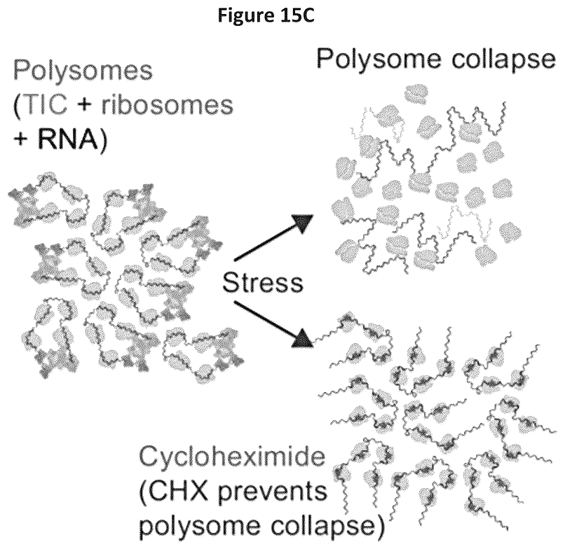

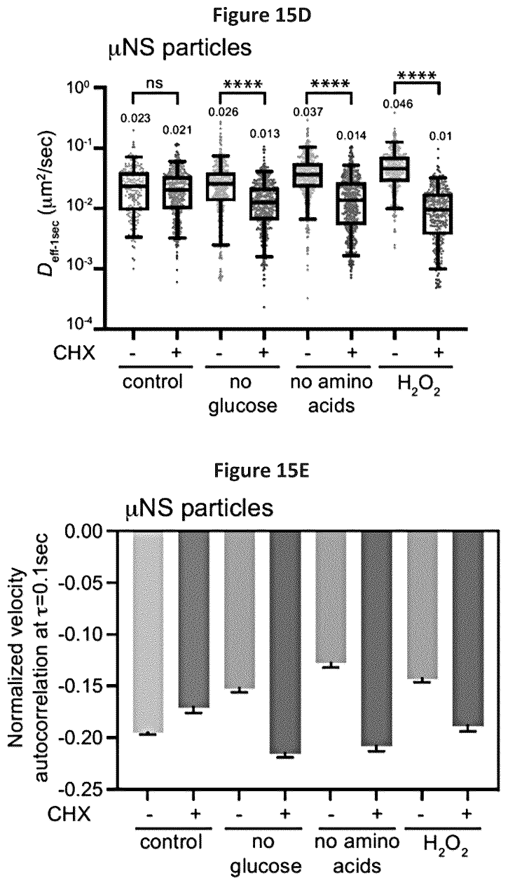

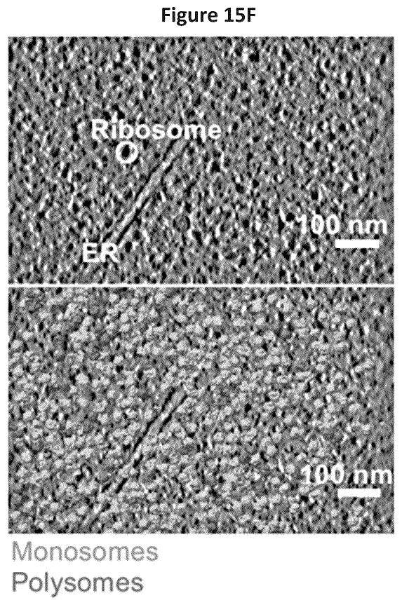

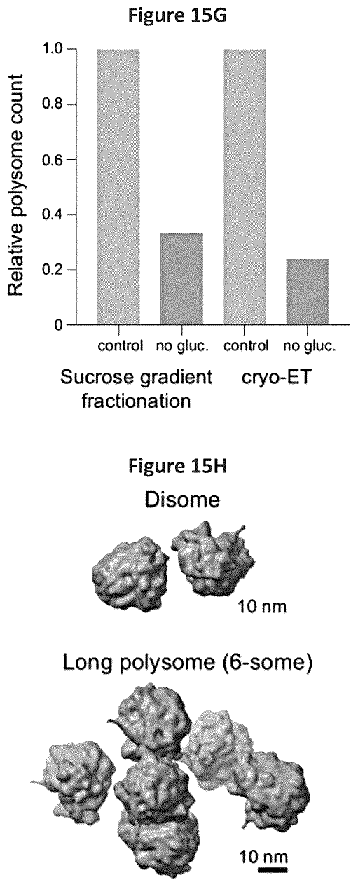

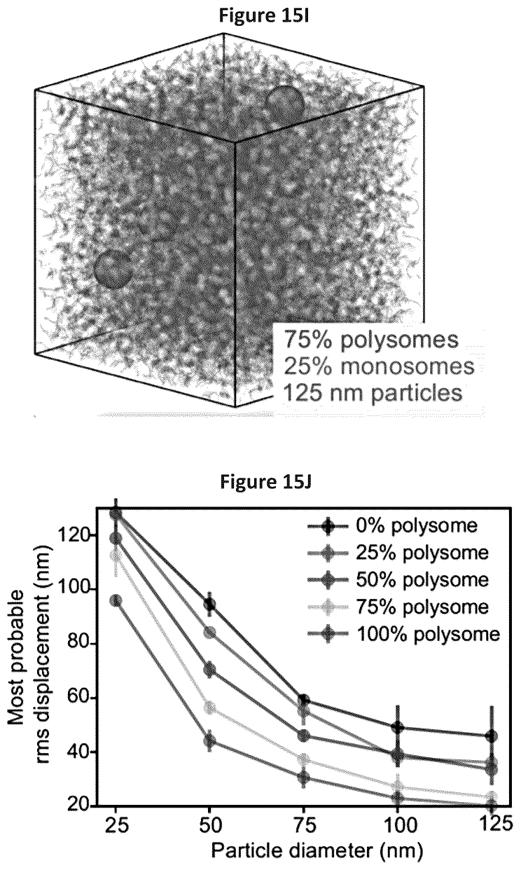

Consistent with previous studies (25), polysome profiling by fractionation of cell extracts on a sucrose gradient indicated a dramatic reduction in the fraction of ribosomes in polysomes within minutes of acute glucose starvation (FIGS. 15A-15B). To test the prediction that preventing polysome disassembly should prevent cytoplasmic fluidization, cells were treated with cycloheximide, which freezes translation elongation, prevents ribosome dissociation from mRNAs, and therefore prevents polysome collapse (57) (FIG. 15A, FIG. 15C). Consistent with the prediction, simultaneous stress and cycloheximide treatment completely abolished the increase in diffusivity of μNS particles (FIG. 15D). By contrast, cycloheximide treatment in glucose rich medium did not change the diffusivity of μNS particles (FIG. 15D), suggesting that inhibition of translation elongation per se did not impact cytoplasmic fluidity, but rather changes in polysome abundance were crucial. Furthermore, preventing polysome disassembly also prevented the reduction of elastic confinement in all tested stresses (FIG. 15E). Therefore, polysome disassembly was crucial for the initial transient increase of mesoscale particle diffusivity and reduction of clastic confinement (FIGS. 15D-15E).

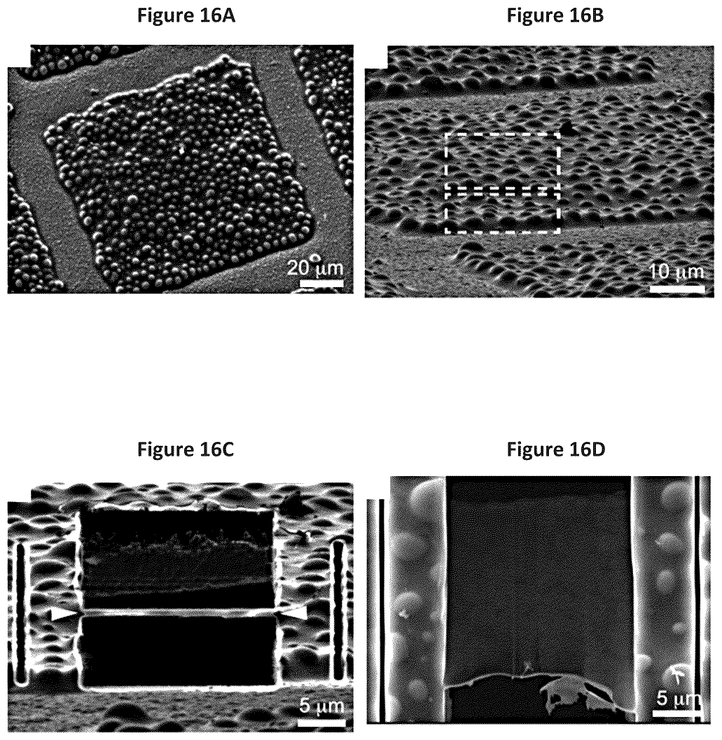

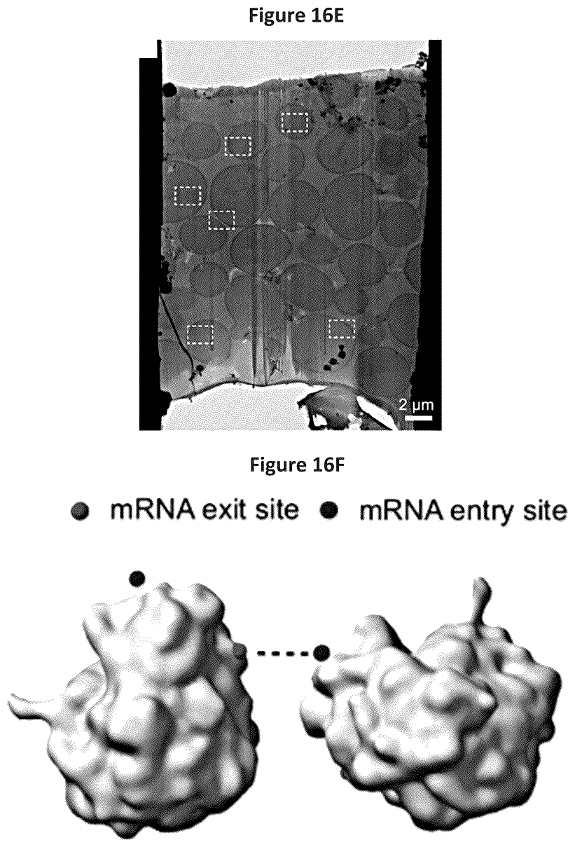

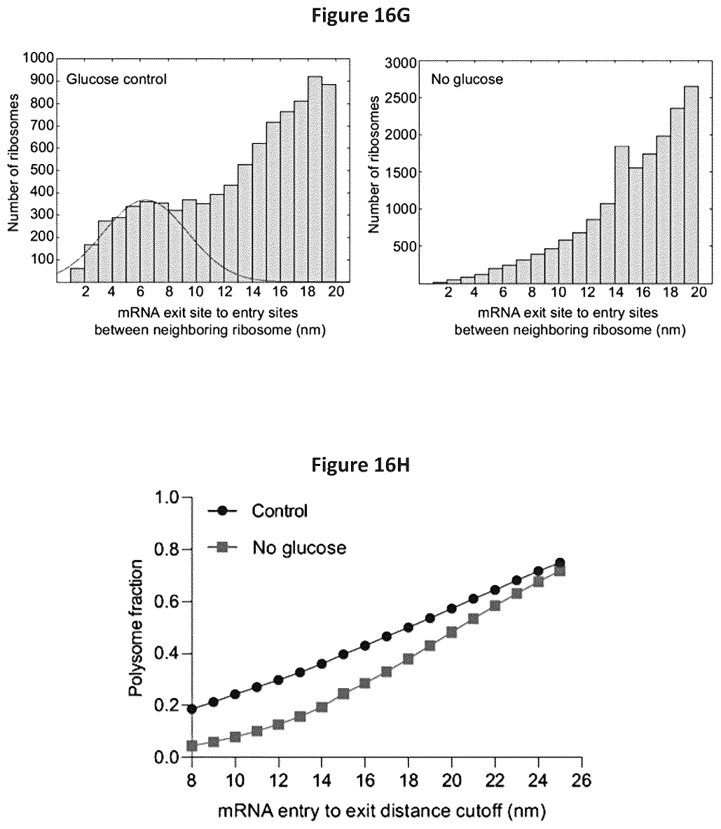

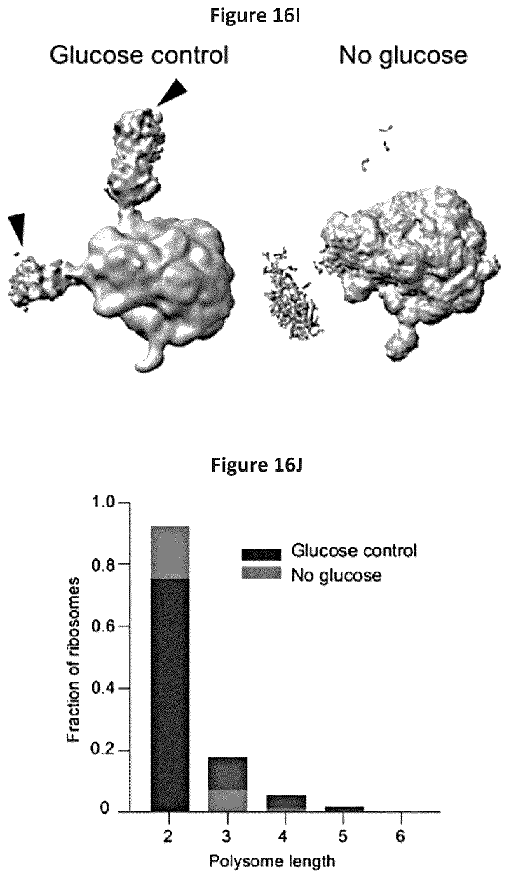

To better understand the spatial constraints imposed by polysomes, their distribution within the cellular context was analyzed based on cryo-electron tomograms (cryo-ET) of intact S. cerevisiae (FIG. 15F, FIGS. 16A-16E) (58,59). To define polysomes, ribosomes were detected in the cryo-ET data of cells in glucose rich and acute glucose starvation conditions and the distance was measured between the mRNA exit and entry sites between adjacent ribosomes, taking their relative orientations into account (FIG. 16F). In glucose rich conditions, the distances exhibited a bimodal distribution that could be fit by two Gaussians, while the peak around smaller distances was largely absent upon glucose starvation. A conservative distance cutoff of 8 nm was used to assign a ribosome to a closely assembled ribosome (FIGS. 16G-16H). This analysis assigned 18.6% of ribosomes to polysomes in glucose rich conditions. After 30 minutes of acute glucose starvation, the polysome fraction was reduced to 4.5%. These values were smaller than those estimated from biochemical fractionation (FIG. 15B) as polysomes with loosely packed arrangements were not included. Therefore, to compare the change in polysome fraction upon glucose starvation determined by sucrose gradient fractionation and the cryo-ET data, the number of polysomes in the starvation conditions were normalized to the respective glucose controls (FIG. 15G). Both experiments showed a similar decrease in polysome fractions with a reduction of 67% based on sucrose gradient fractionation and 76% based on the cryo-ET data. Consistently, maps generated by subtomogram averaging of the ribosomes exhibited clear densities at the mRNA exit and entry sites under the glucose rich conditions that were indicative of polysomal arrangement, and which were not present in the starvation condition (FIG. 16I). Furthermore, the size of polysomes decreased under glucose starvation (FIG. 15H, FIG. 16J). The overall reduction in polymeric crowders and shift to smaller polysome sizes under glucose starvation was consistent with the present hypothesis that a disassembly of polysomes due to short-term glucose starvation promotes mesoscale fluidization. Cryo-EM data collection, refinement and validation statistics are shown in Table 3.

TABLE 3
Cryo-EM data collection, refinement, and validation statistics.

| | | Subtomogram average |
|---|---|---|
| | Subtomogram average | of 80S ribosomes in |
| | of 80S ribosomes in | S. cerevisiae under |
| | native S. cerevisiae | acute glucose starvation |
| Magnification | 26,000 | 26,000 |
| Voltage (kV) | 300 | 300 |
| Electron exposure | 114.7 | 133.4 |
| (e−/Å²) | | |
| Defocus range (μm) | −2.0 to −4.0 | −2.0 to −4.0 |
| Pixel size (Å) | 6.85 (2x binning) | 3.425 |
| Symmetry imposed | C1 | C1 |
| Initial particle images | 22,498 | 53,596 |
| (no.) | | |
| Final particle images | 22,433 | 51,821 |
| (no.) | | |
| Map resolution (Å) | 16.2 | 10.2 |
| FSC threshold | 0.143 | 0.143 |

A coarse-grained molecular dynamics (CGMD) simulation was then developed to test whether the presence of polysomes could be sufficient to explain the changes in mesoscale particle dynamics that were observed experimentally (FIG. 15I). The simulation was parameterized based on the fraction of polysomes and monosomes determined by sucrose gradient fractionation (FIG. 15B) and the 30% excluded volume fraction in the cytoplasm measured in (21). Polysomes were represented as a chain of six connected ribosomes, to match the median polysome length measured in sucrose gradient ribosome profiles (FIG. 15A). The effect of changes in polysome fraction on the effective diffusivity of particles of various sizes was then modeled, and it was found that increasing the fraction of polysomes reduced mesoscale particle diffusivity in a size-dependent manner (FIG. 15J), consistent with the present experimental findings.

### Example 7. Condensation of RNA into Stress Granule or Processing Bodies is Required for Cytoplasmic Fluidization

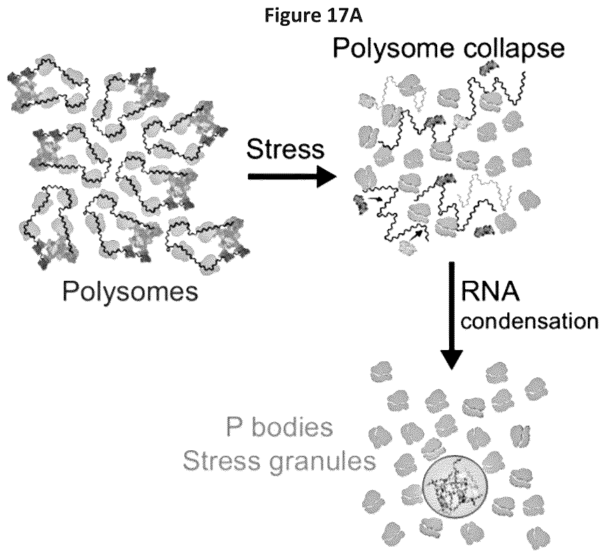

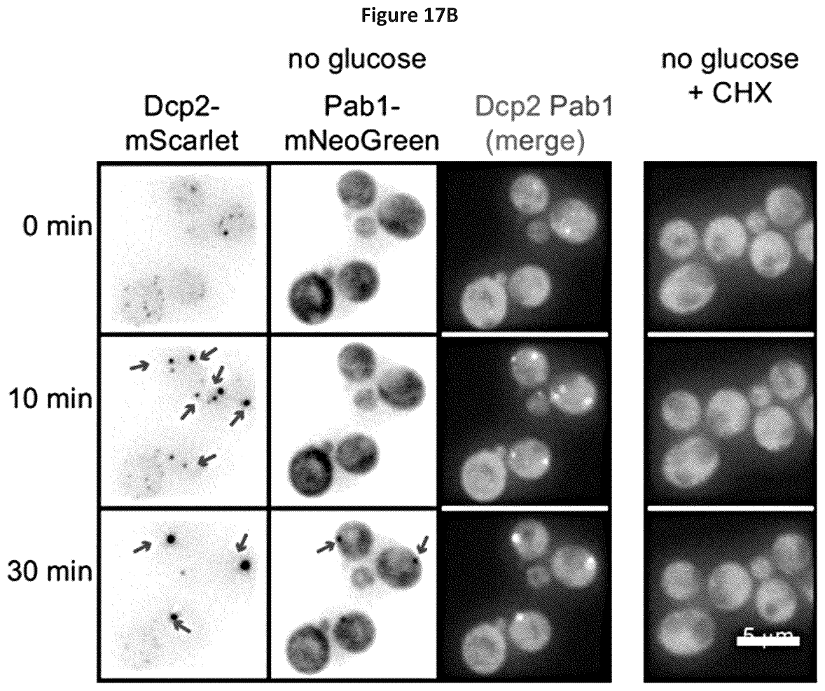

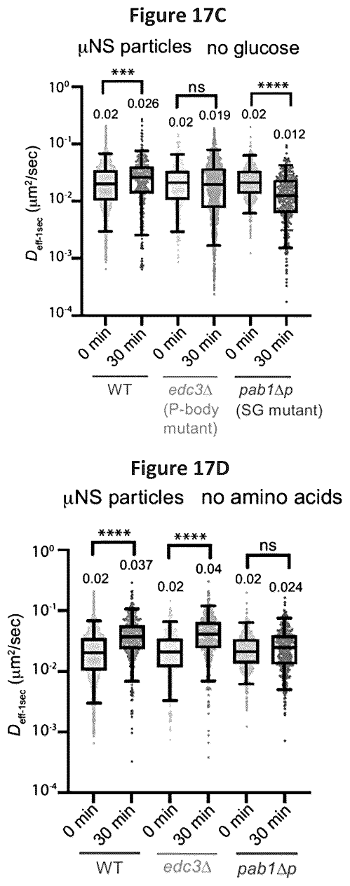

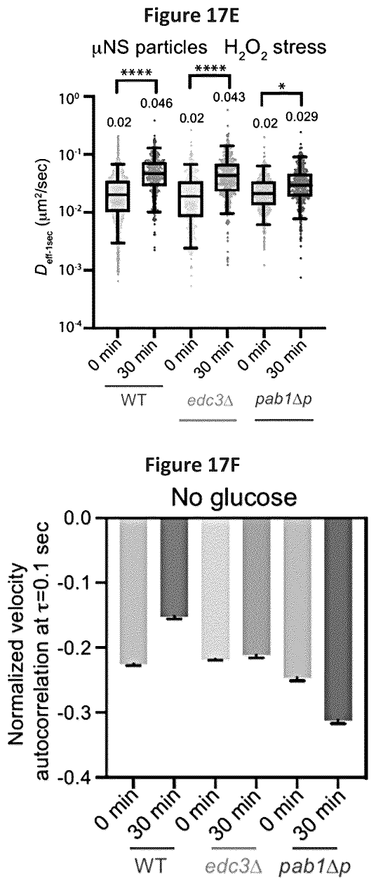

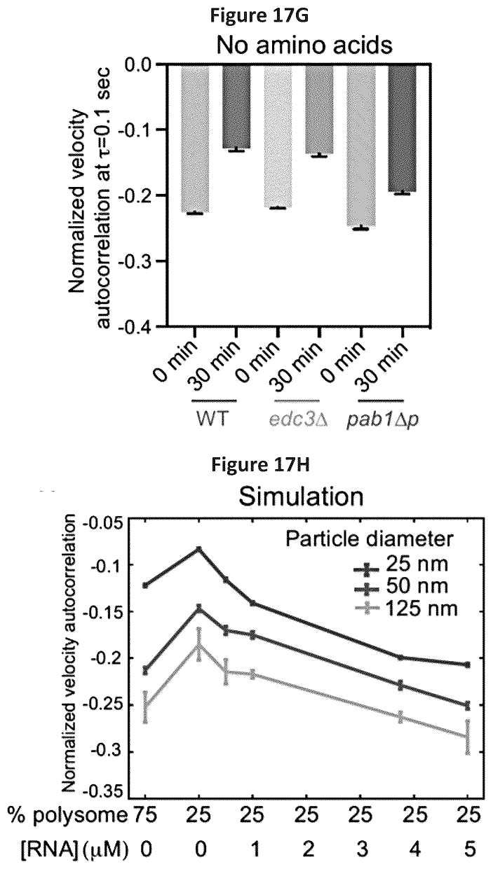

Upon polysome disassembly, mRNAs are released. These mRNA molecules can associate with RNA binding proteins that drive the formation of stress induced condensates, including processing bodies (P-bodies) and stress granules (60-62) (FIG. 17A). The release of mRNA and formation of these condensates is blocked by cycloheximide (63). It was hypothesized that, upon release from polysomes, free mRNA molecules could contribute to interactions that increase the elasticity of the cytoplasm, and condensation of mRNAs might reduce these interactions and thereby reduce elastic confinement.

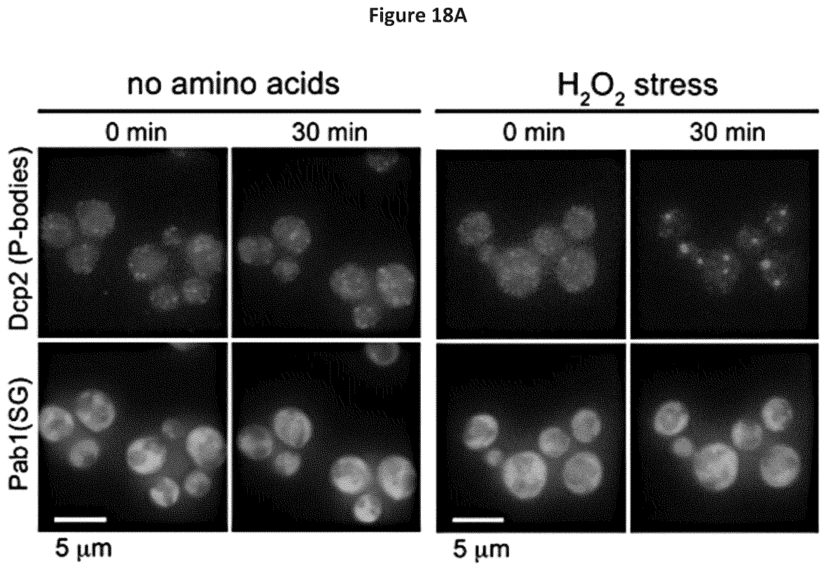

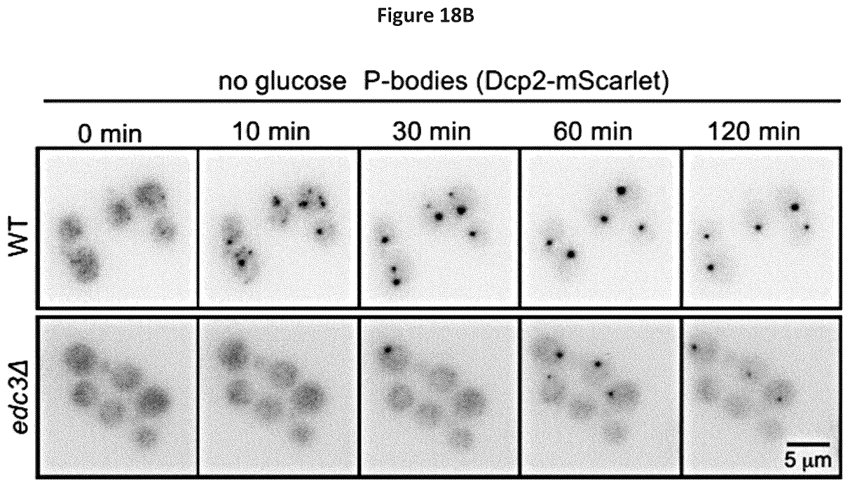

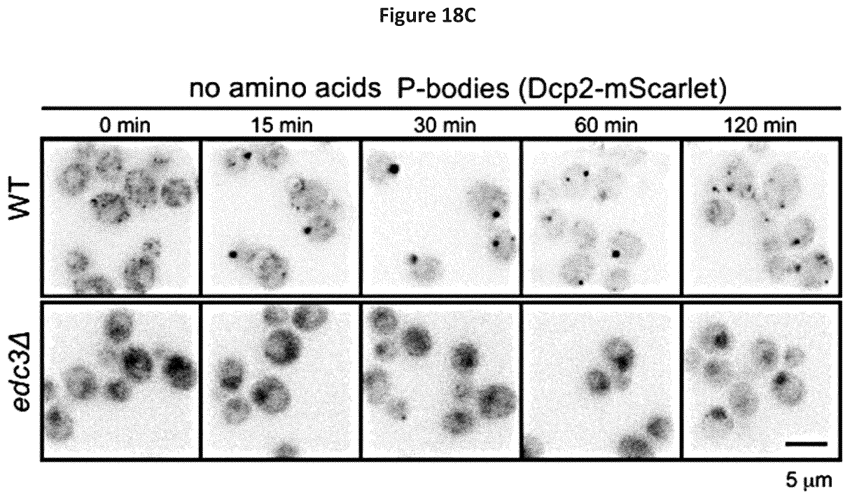

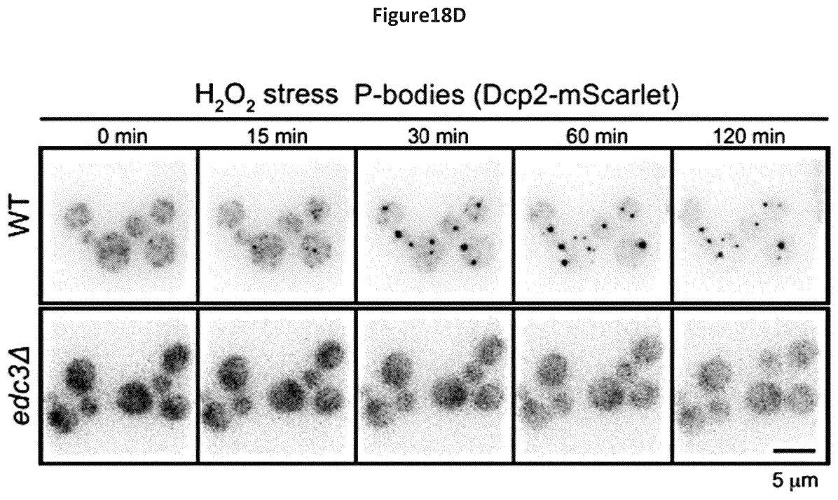

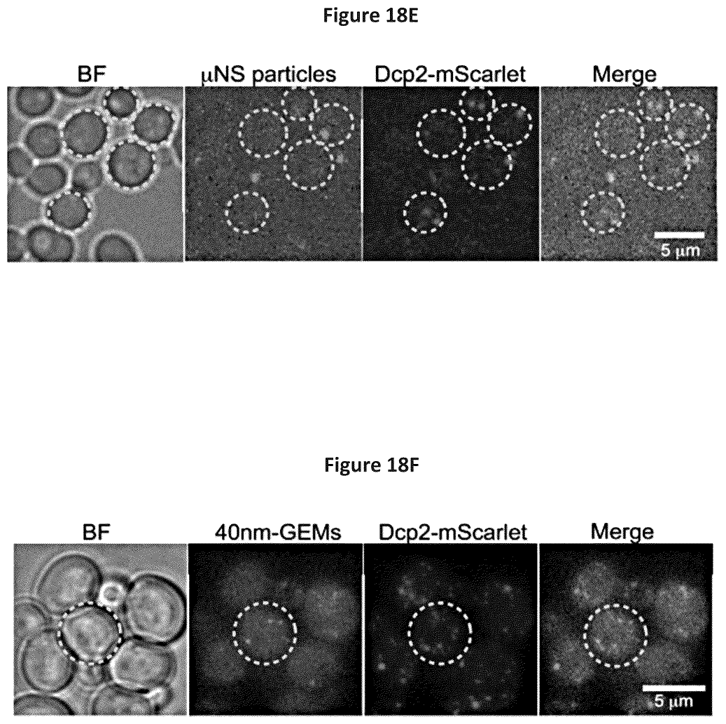

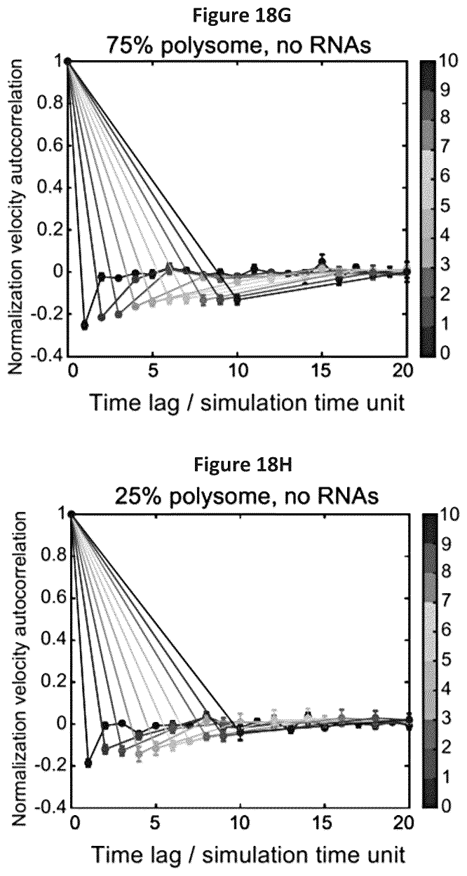

To test this hypothesis, the formation of RNP granules in cells challenged by stresses was monitored through live-cell imaging of the Dcp2 and Pab1 proteins, which are markers for P-bodies and stress granules respectively (FIG. 17B). Consistent with reports (60), P-bodies formed rapidly, becoming clearly visible 10 minutes after acute glucose starvation, while visible stress granules appeared more slowly and were less prominent (FIG. 17B). Treatment with cycloheximide completely inhibited the formation of both P-bodies and stress granules (FIG. 17B). On the other hand, Dcp2 foci but not Pab1 foci were observed in both amino acid starvation and oxidative stress (FIG. 18A), suggesting that P-bodies were formed in these conditions, and that if stress granules were formed, they were diffraction-limited and not readily detected by conventional light microscopy (31,34).

Since the formation of RNP granules correlated with cytoplasmic fluidization, it was tested if these structures were required for this physical change. The Edc3 protein is a key P-body scaffold (64-66). It was found that an EDC3 gene deletion mutant (ede3Δ) failed to efficiently form P-bodies in all tested stresses, as indicated by a marked reduction in the appearance of Dcp2 foci (FIGS. 18B-18D). To inhibit stress granule formation, pab1ΔP mutant cells were used (67). The low complexity P domain has been demonstrated to facilitate Pab1 protein phase separation in vitro, and pab1ΔP mutant cells have reduced cellular fitness in heat stress and energy depletion stress (67). First, it was confirmed that neither rheological probe was sequestered into P-bodies upon acute glucose starvation (FIGS. 18E-18F), indicating that they were reliable reagents for defining the cytosolic environment outside of P-bodies. Almost no increase of μNS particle diffusivity in edc3Δ mutant cells and a decrease of μNS particle diffusivity in pab1ΔP was observed at the initial 30 min of glucose starvation (FIG. 17C). Analysis of the normalized velocity autocorrelation function also showed less change in elastic confinement in these mutants (FIG. 17F). Furthermore, pab1ΔP abrogated or strongly attenuated mesoscale fluidization in both amino acid starvation and H₂O₂ treatment (FIGS. 17D-17G), highlighting a crucial role for stress granules assembly in driving mesoscale fluidization of the cytoplasm in these stresses. Thus, P-bodies were required for cytoplasmic fluidization in carbon starvation, and stress granules were required for cytoplasmic fluidization in all stresses that were tested.

The experimental data herein strongly supported a model in which both polysomes and mRNAs in the cytosol can contribute to clastic confinement that limits mesoscale diffusivity. To independently test the biophysical role of polysome concentration and free mRNA, the present simulation framework was built upon. As a minimal change to the model herein, free RNA were included as spherical particles that have a weak nonspecific interaction with ribosomes. To parameterize the simulation, polysome and RNA concentrations were chosen based on present measurements and those reported in the literature (FIG. 15B). Specifically, it has been reported that there are 15,000-60,000 mRNAs per yeast cell (68,69), and the yeast cytoplasmic volume is around 25 μm³ (FIG. 13D) which corresponds to a cytoplasmic mRNA concentration of 1-4 μM. The normalized velocity autocorrelation function was measured to infer elastic confinement in the present simulations. 125 nm particles displayed negative velocity autocorrelations that were most apparent at short time-scales (FIGS. 18G-18I). Consistent with the experimental results herein, it was found that both high polysome fractions and the presence of free RNA increased clastic confinement of these mesoscale particles (FIG. 17H). Comparison of the brightness of the 40 nm-GEMs and 50 nm-GEMs showed that the 50 nm-GEMs were significantly brighter and this made them more widely useful with less specialized microscopes (FIG. 3J).

### Example 8. Increased Mesoscale Diffusivity Facilitates Q-Body Formation Upon Acute Glucose Starvation

Multiple types of membraneless organelle form in stress conditions (13,29,30,70,71), often by an initial nucleation of multiple small assemblies that then fuse together to form the mature organelle. This growth by coalescence can be inhibited by crowded or elastic confined environments because subassemblies reach a size at which they no longer diffuse efficiently to encounter one another and fuse. For example, the growth of synthetic condensates is mechanically inhibited by chromatin (72), and the cytoplasm has been shown to have elastic properties that frustrate the growth of synthetic mesoscale condensates (18). It was therefore hypothesized that the fluidization of the cytoplasm by polysome disassembly and RNP granule formation could facilitate the formation of other mesoscale membraneless organelles.

Q-bodies are membraneless organelles that form upon glucose starvation or heat shock. These acute stresses abruptly terminate protein translation, likely leading to accumulation of nascent peptides that may misfold (73). At the same time, a sudden drop of ATP levels can cause formation of protein aggregates in cells (14,74). The chaperone Hsp42 has been demonstrated to associate with misfolded proteins and promote Q-body formation. Deletion of the HSP42 gene reduces Q-body assembly and reduces cellular fitness when yeast cells are challenged by heat stress or glucose starvation (11,14), indicating that Q-bodies help cells adapt to stress.

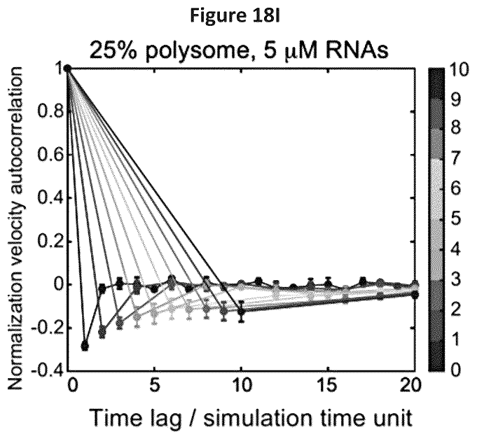

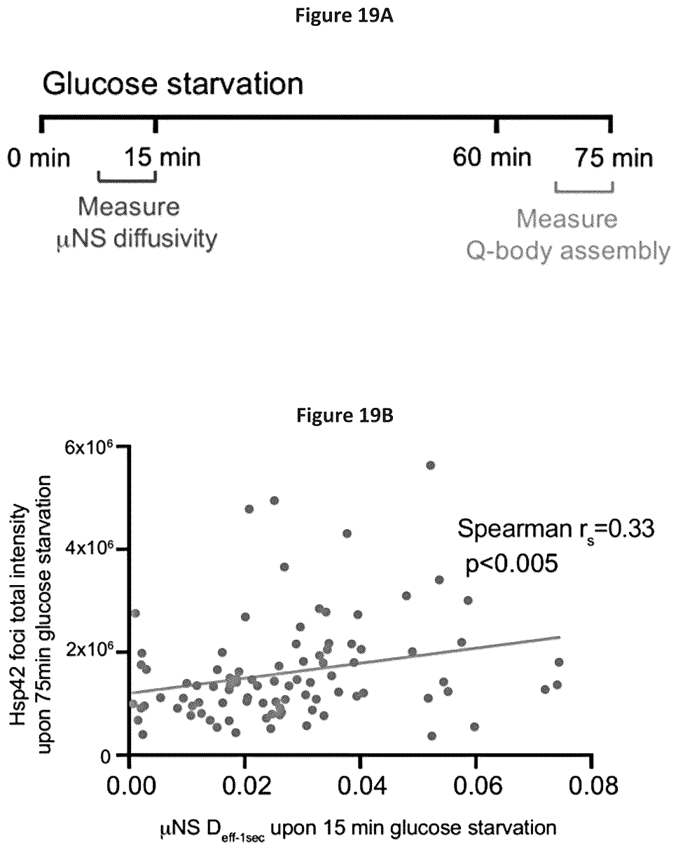

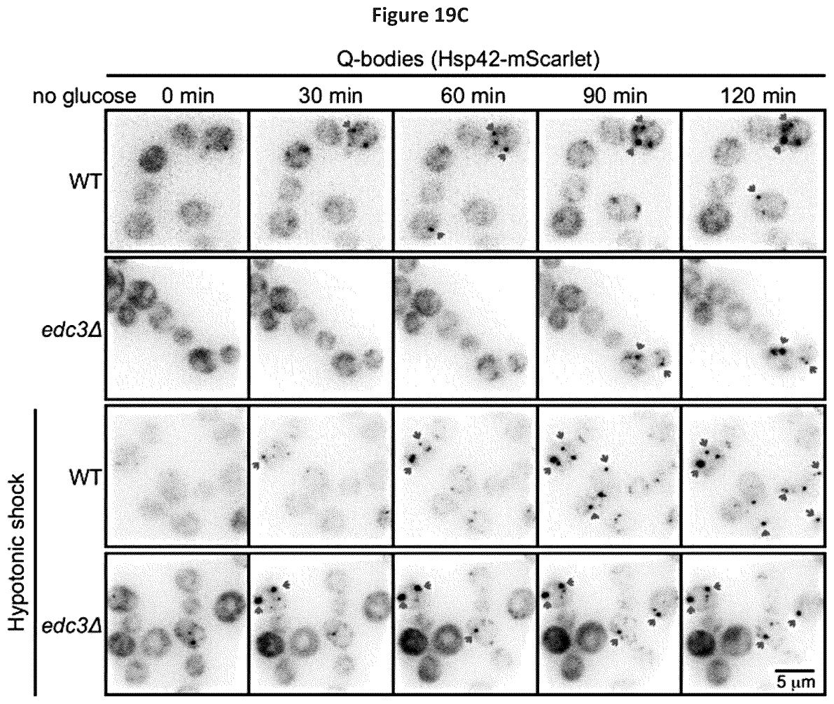

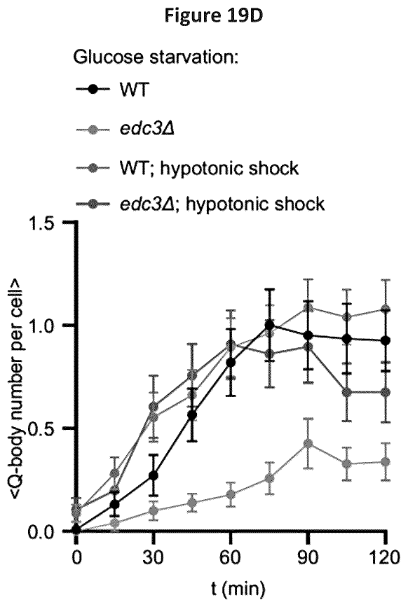

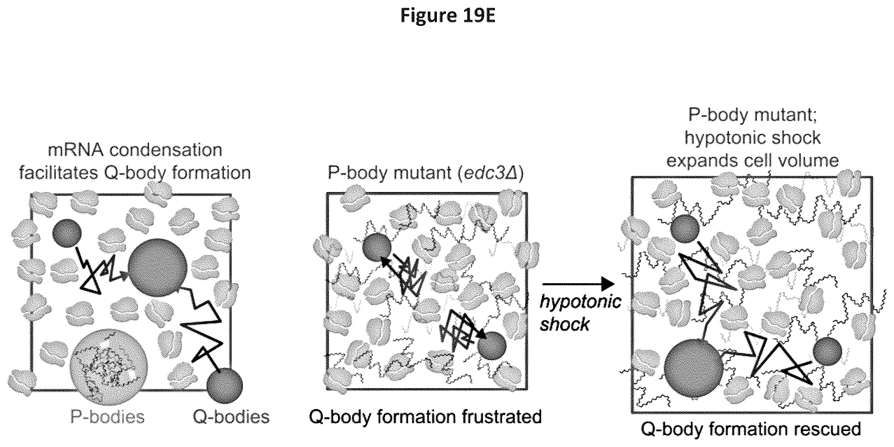

It was tested if transiently increased mesoscale diffusivity upon glucose starvation was important for efficient Q-body assembly. Endogenously tagged Hsp42-mScarlet was used to indicate Q-body assembly, and μNS diffusivity 15 min after glucose starvation and the subsequent intensity of Hsp42 foci 75 min after glucose starvation were measured in the same cells. Significant positive correlation was found between early cytoplasmic fluidization and the intensity of Hsp42 foci (FIGS. 19A-19B). These results suggest that cytoplasmic fluidization may enable Q-body assembly.

Q-body formation was then compared upon acute glucose starvation between wild type and edc3Δ (P-body deficient) mutants (FIGS. 19C-19D). In wild type cells, it was found that the average number of Hsp42-mScarlet foci increased over the first hour of glucose starvation until, on average, all cells had one Q-body (FIG. 19D, black line) Thus, Q-body formation occurred during the period of transient fluidization of the cytoplasm after glucose starvation. Strikingly, Q-body formation was significantly attenuated in the edc3Δ mutants that do not effectively fluidize their cytoplasm (FIGS. 19C-19D, orange line), consistent with cytoplasmic fluidization enabling formation of mesoscale Q-bodies.

An alternative hypothesis is that Edc3 or P-bodies were required for Q-body formation through a mechanism independent of cytoplasmic fluidization. If physical fluidization of the cytoplasm is the key defect in edc3Δ cells, orthogonal methods to fluidize the cytoplasm should rescue Q-body formation. It was possible to decrowd the cytoplasm by increasing cell volume through acute hypotonic shock (21). To achieve hypotonic shock, wild type and edc3Δ mutant yeast cells were cultured in hypertonic media (media supplemented with 500 mM KCl as an osmolyte) overnight. In these conditions, cells activated the high-osmolarity glycerol (HOG) pathway to accumulate osmolytes (primarily glycerol), thereby osmotically balancing the cell interior enabling growth (75). Suddenly shifting these cells to media without excess osmolyte (i.e., without KCl) created a hypotonic shock because the osmolytes that have accumulated in the cell take time to be degraded and exported (76). This hypotonic shock caused water to enter the cell, leading to a sudden increase in volume and a corresponding decrease in macromolecular crowding, thereby increasing mesoscale diffusivity (21).

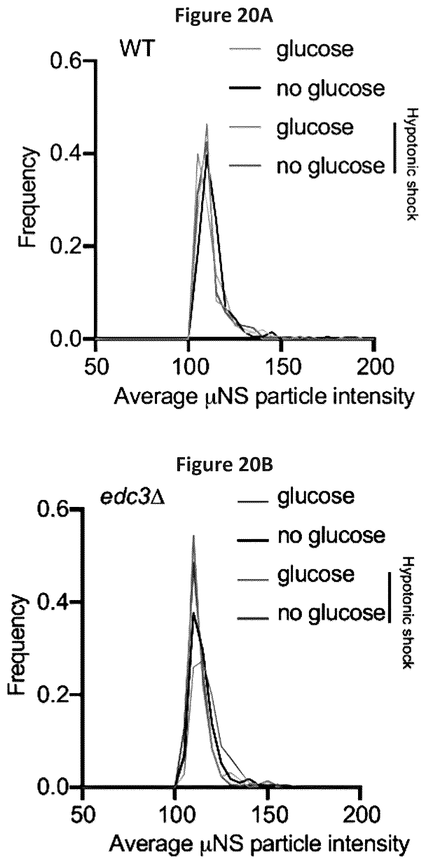

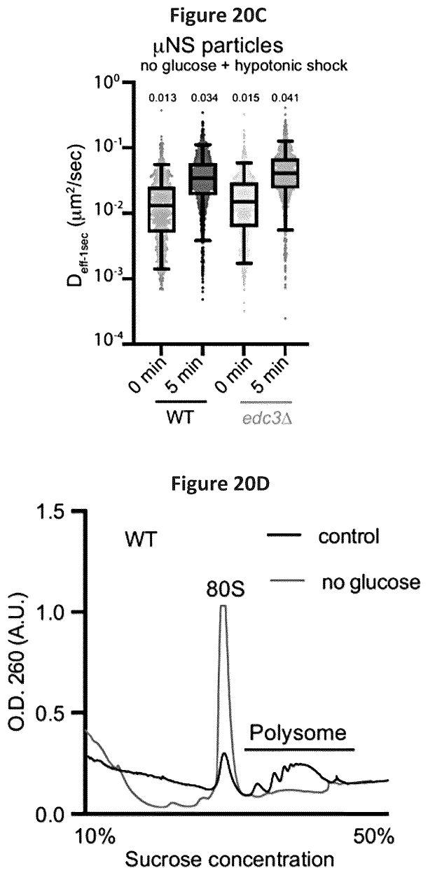

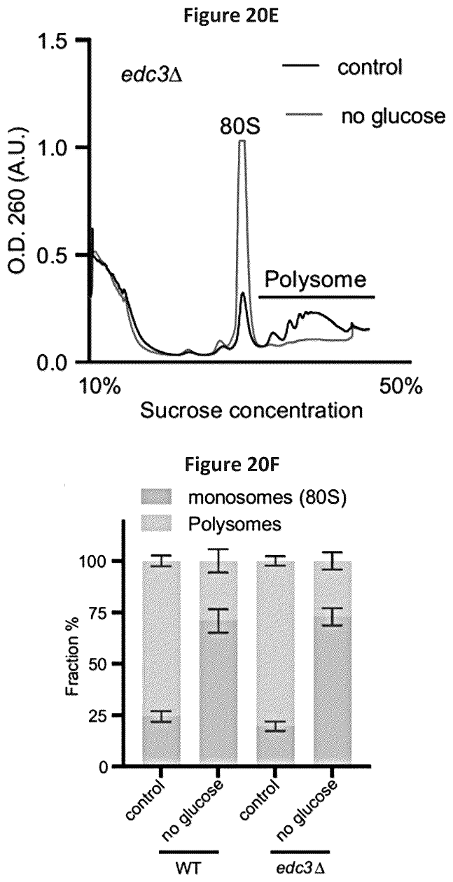

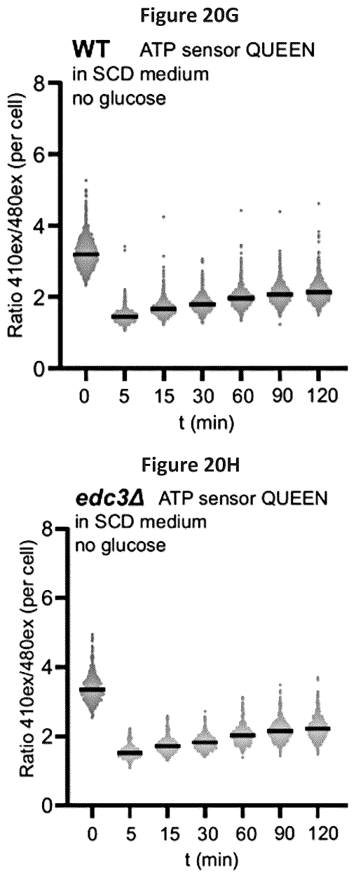

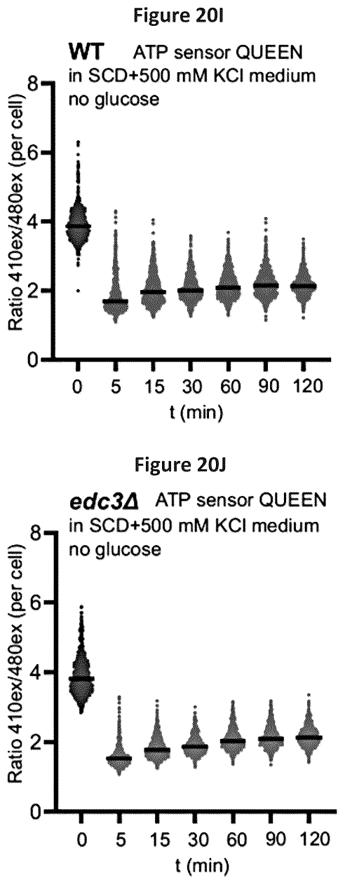

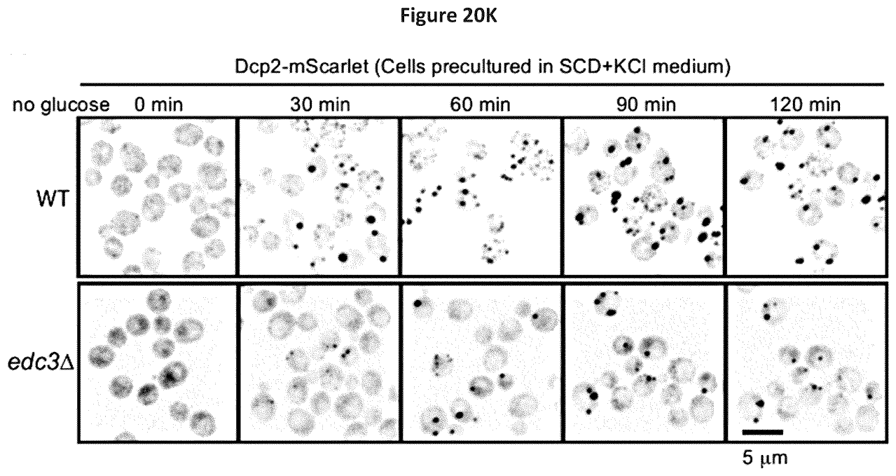

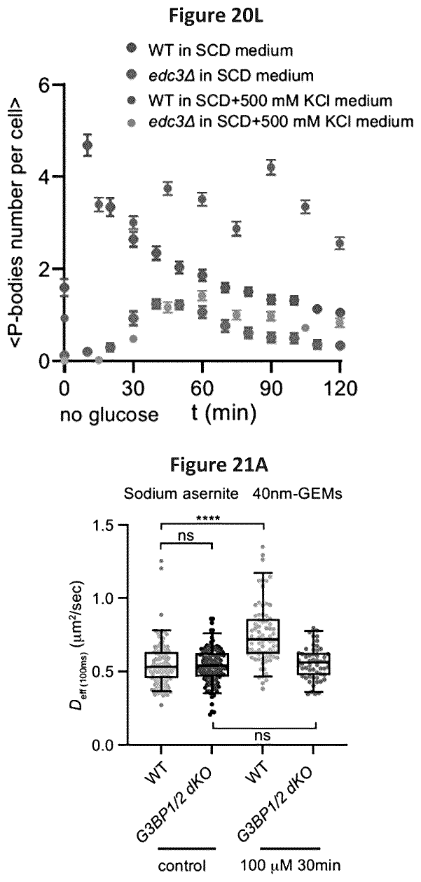

Upon hypotonic shock, the diffusivity of UNS particles increased drastically at 5 minutes in both wild type and edc3Δ mutant cells, without changing the size of μNS particles (FIGS. 20A-20C). Consistent with the hypothesis, experimentally-induced decrowding of the cytoplasm was sufficient to completely rescue Q-body assembly in edc3Δ cells (FIGS. 19C-19D, red line).

It was confirmed that there was no translation defect in edc3Δ cells in normal growth conditions, and a similar reduction of polysomes was observed in edc3Δ cells compared to wild type yeast in glucose starvation (FIGS. 20D-20F), consistent with reports (64). Based on these observations, the reduction of microscopically visible P-bodies in an edc3Δ mutant was not due to defects in translational repression, but rather due to the failure to condense untranslated mRNAs into P-bodies. Furthermore, neither hypotonic shock nor deletion of EDC3 affected ATP levels over the course of these experiments; the same degree and kinetics of ATP reduction was observed during starvation compared to WT cells (FIGS. 20G-20J). Finally, it was confirmed that the P-body assembly defect was still present in ede3Δ cells upon glucose starvation in the hypotonic shock condition (FIGS. 20K-20L). These results supported the model in which P-body formation increased mesoscale particle diffusivity, facilitating the formation of mesoscale Q-body condensates during the initial response to glucose starvation (FIG. 19E).

### Example 9. Stress Granule Formation Increases Mesoscale Diffusivity in Mammalian Cells

Experiments in yeast showed that the formation of stress granules was important for mesoscale cytoplasmic fluidization in response to multiple stresses. It was tested if similar phenotypes could be observed in U2OS mammalian cells. Stress granules were induced in response to many stresses and were widely implicated in physiological and pathological conditions in mammalian cells (29,77-80). G3BP1 and G3BP2 proteins are important scaffolds for stress granule formation that interact with both RNA and RNA binding proteins (79,80). Cells with a double knock out of G3BPI and G3BP2 (G3BP1/2 dKO) were previously demonstrated to prevent stress granule formation in some types of stress such as sodium arsenite but not others, such as heat stress (79). Therefore, it was analyzed how mesoscale diffusivity was impacted by sodium arsenite treatment and 45° C. heat stress. Note that the μNS particles that were used as rheology probes in yeast cells were originally derived from a vertebrate viral protein (81), and so it was not certain that these could be used as passive orthogonal probes in mammalian cells; therefore, the mammalian experiments were restricted to GEM nanoparticles. There was an increased variance of the median diffusivity of 40 nm-GEMs between control cells after heat shock that was not apparent in G3BP1/2 dKO cells (FIGS. 21A-21B). These results suggested that stress granules impact the physical properties of cells in complex ways in the context of heat shock that would require further study to understand. On the other hand, it was found that the diffusivity of 40 nm-GEMs increased after 30 min of oxidative stress induced by 100 μM sodium arsenite in control cells but not G3BP1/2 dKO cells (FIGS. 21A-21B), consistent with the hypothesis that formation of stress granules leads to cytoplasmic fluidization upon oxidative stress.

In general, external stresses lead to translational inhibition and therefore induce stress granule formation. However, stresses such as heat shock may have pleiotropic effects. Therefore, to more specifically investigate the effects of stress granules on the physical properties of the cytoplasm, translation inhibitors were used (FIG. 21C). Translation inhibitors that cause collapse of polysomes and release of mRNA (i.e., puromycin causes premature chain termination, and DMDA-patcamine A (DMDA PatA) inhibits translation initiation factor eIF4A) (82-85), induced widespread formation of stress granules as indicated by condensation of the stress granule protein G3BP1 (FIG. 22A). In contrast, treatment with the translation inhibitor cycloheximide, which prevents polysome disassembly (57), did not lead to stress granule formation (FIG. 22A). Moreover, pretreatment with cycloheximide before applying puromycin or DMDA PatA suppressed stress granule formation (FIG. 22A), supporting the idea that release of mRNAs from polysomes seeds the formation of stress granules.

The spatial distribution of mRNAs after triggering stress granule formation with DMDA-PatA was analyzed using poly-dT FISH. In U2OS control cells, clear poly-dT foci in the cytoplasm were observed after DMDA-PatA treatment, consistent with mRNA condensation into stress granules (FIG. 21D). These poly-dT clusters were absent in G3BP1/2 dKO cells (FIG. 21D, FIGS. 22B-22C). Together, these results supported the model that mRNA released from polysomes upon translation inhibition condenses into stress granules in mammalian cells.

Upon puromycin and DMDA-PatA treatment, 40 nm-GEMs showed an increase of diffusivity in control U2OS cells but not in G3BP1/2 dKO cells (FIGS. 21E-21F, FIG. 22D). Cycloheximide pretreatment of cells suppressed this effect (FIGS. 21E-21F, FIG. 22D). Therefore, conditions that lead to formation of stress granules cause increased mesoscale particle diffusivity, while preventing stress granules formation abolishes this physical change.

The investigation was expanded to explore the effects of stress granule formation on mesoscale diffusivity. Stress granule formation in mammalian cells can be triggered either through eIF2 phosphorylation (e.g., after sodium arsenite treatment) or inhibition of eIF4A helicase (e.g., DMDA PatA is an inhibitor of eIF4A). A small molecule named ISRIB, has been developed as an inhibitor of the integrated stress response. ISRIB inhibited stress granule formation by modifying the effects of eIF2 phosphorylation (86) (FIG. 21G). However, ISRIB failed to prevent stress granule formation upon inhibition of eIF4A helicase activity (87) (FIG. 21G). As predicted by the model that stress granule formation leads to fluidization of the cytoplasm, addition of ISRIB suppressed the increase in diffusivity upon sodium arsenite treatment, but not upon DMDA PatA treatment in control cells (FIG. 21H). By contrast, the physical changes were not observed in G3BP1/2 dKO cells (FIG. 211). These results further strengthened the present model that stress granule formation functions to fluidize the cytoplasm, and highlighted a new potential function for ISRIB in the modulation of the physical properties of the cytoplasm.

### Example 10. Degradation of RNA by Innate Immune Pathways Increases Mesoscale Diffusivity in the Cytoplasm

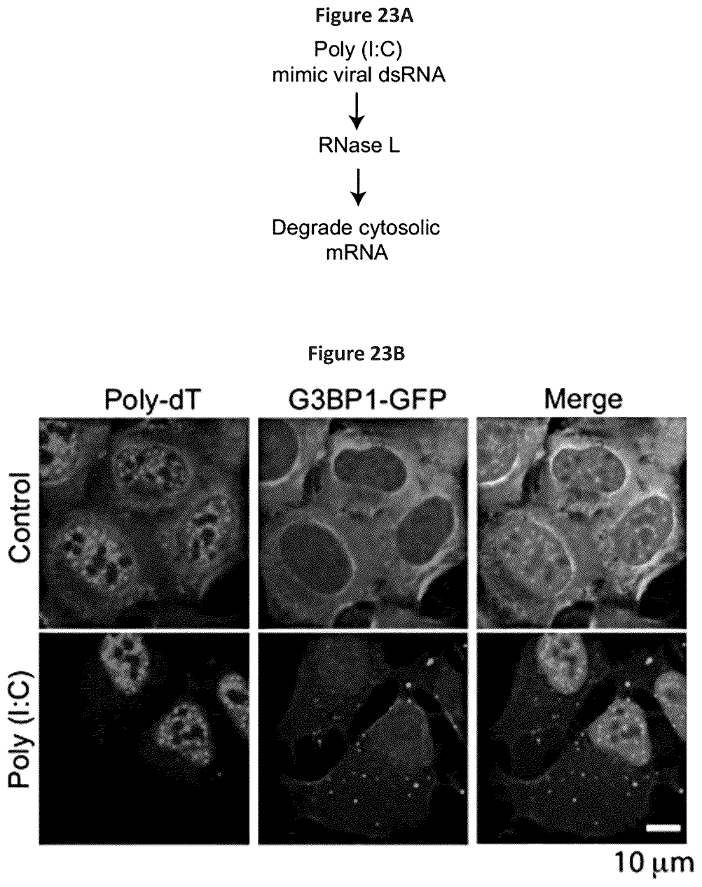

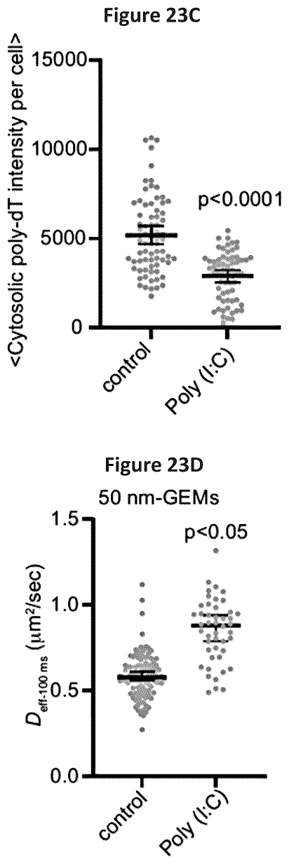

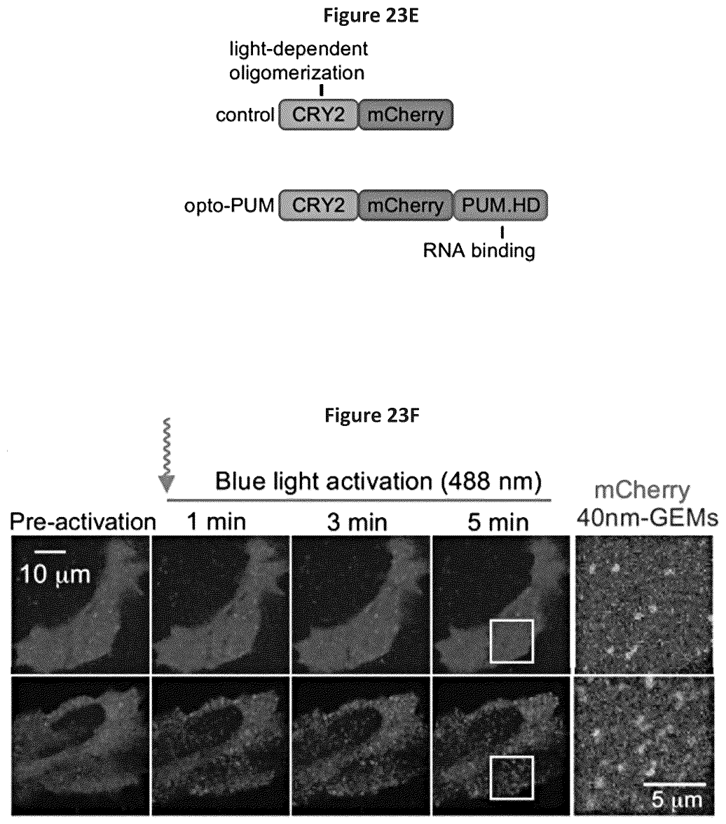

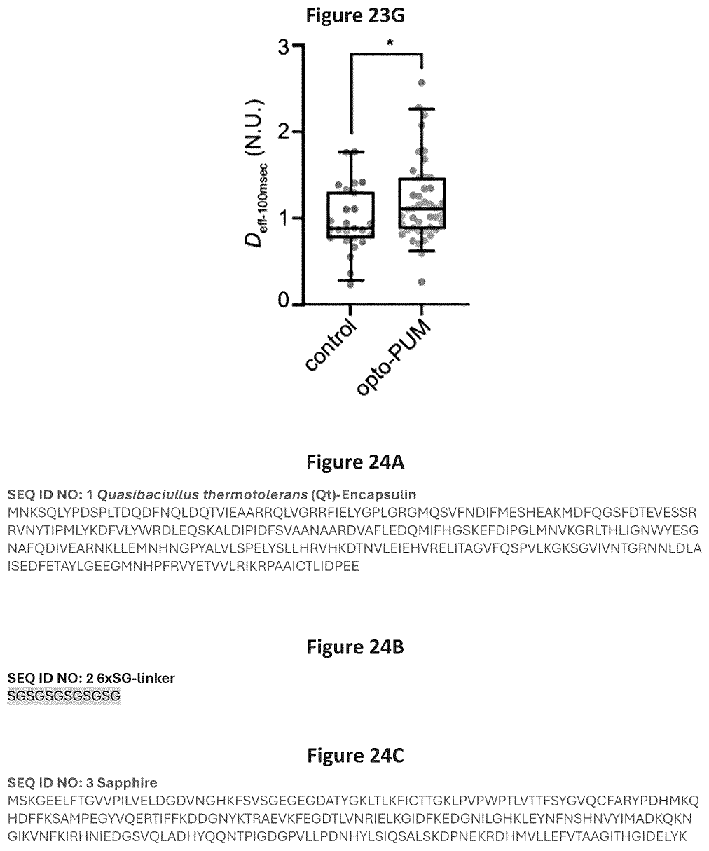

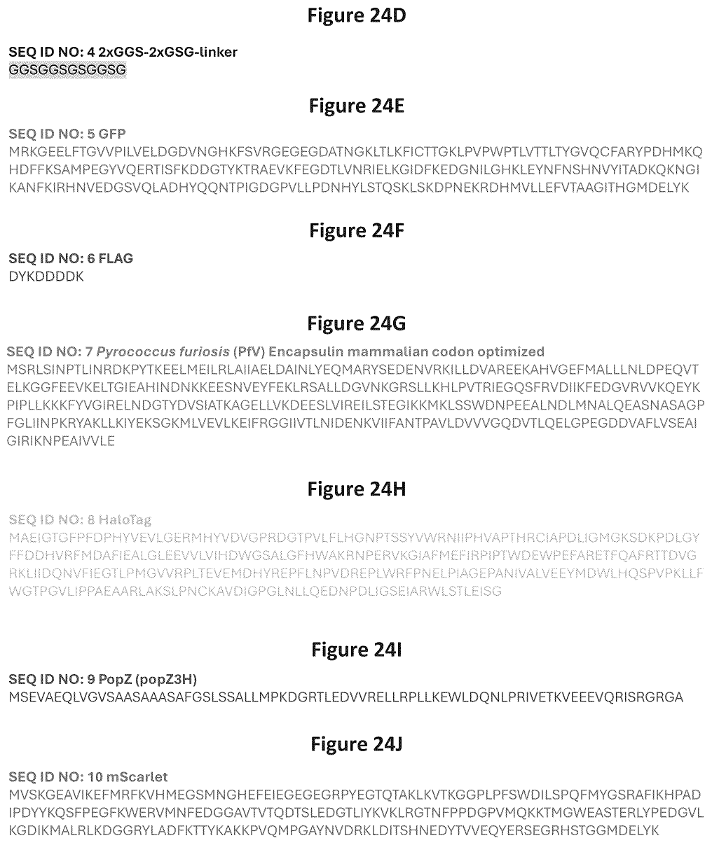

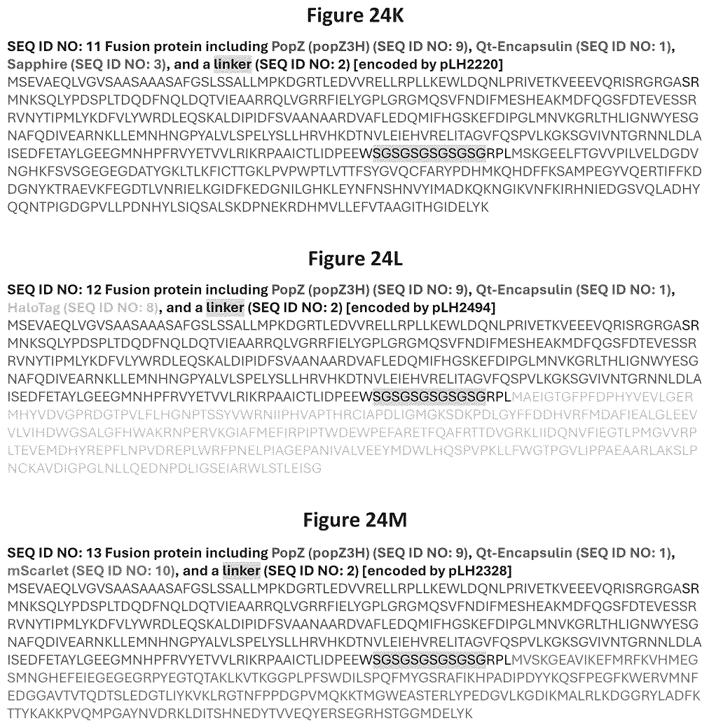

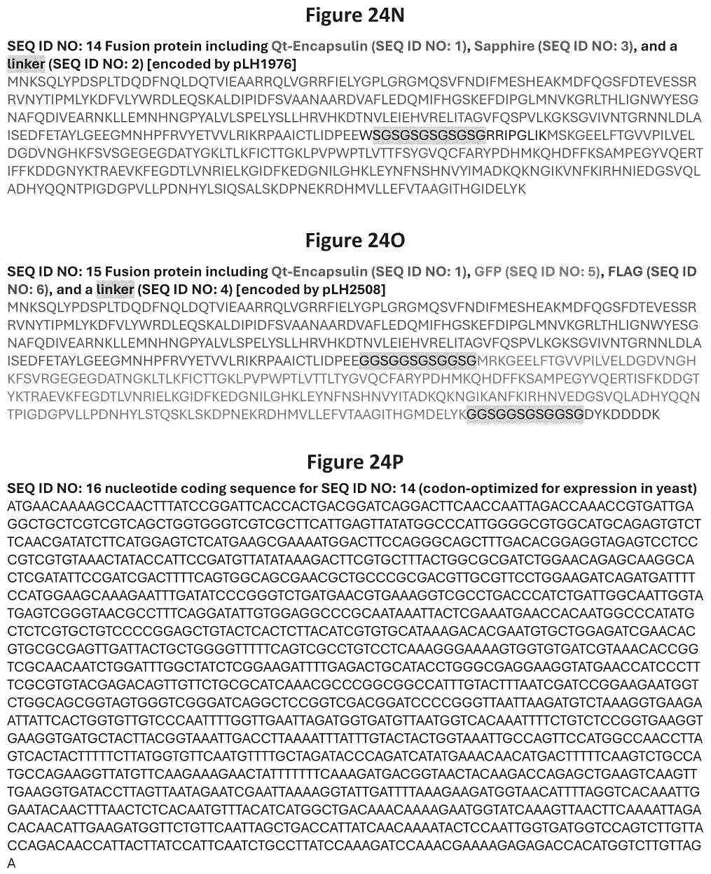

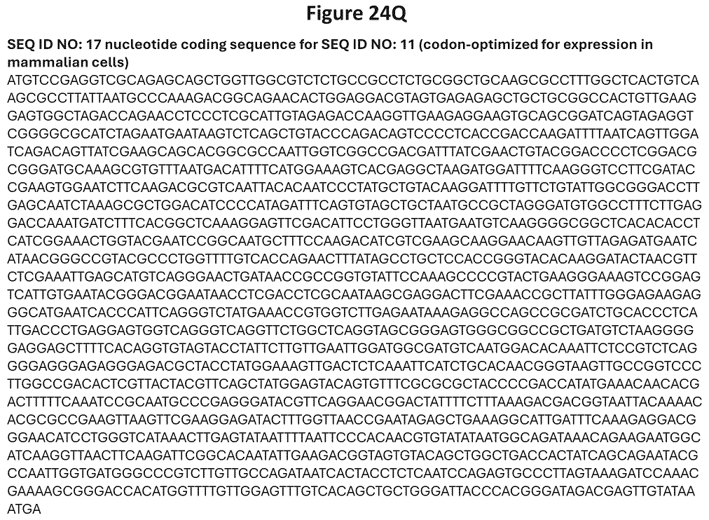

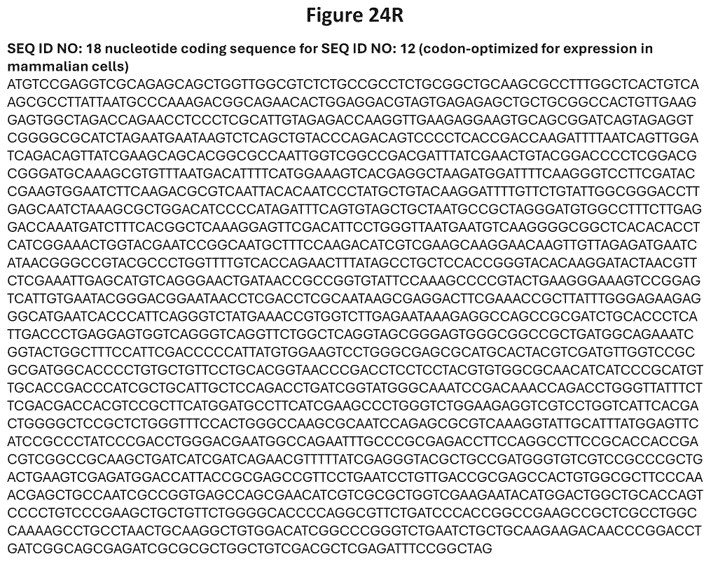

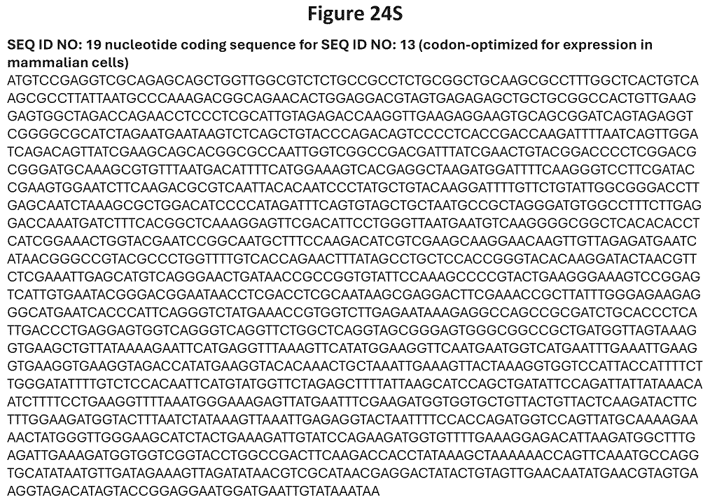

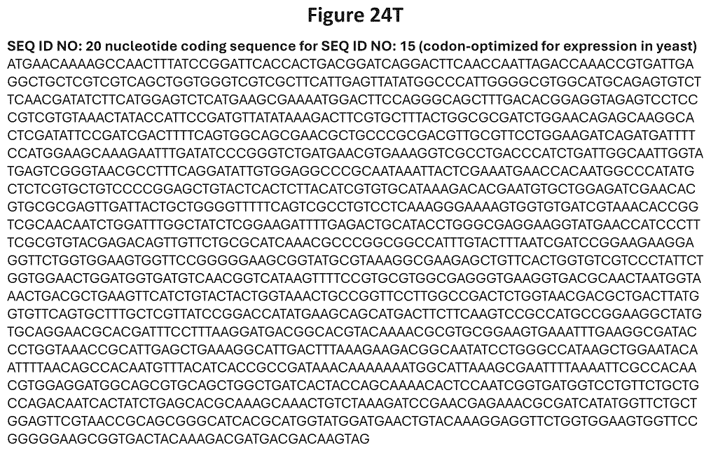

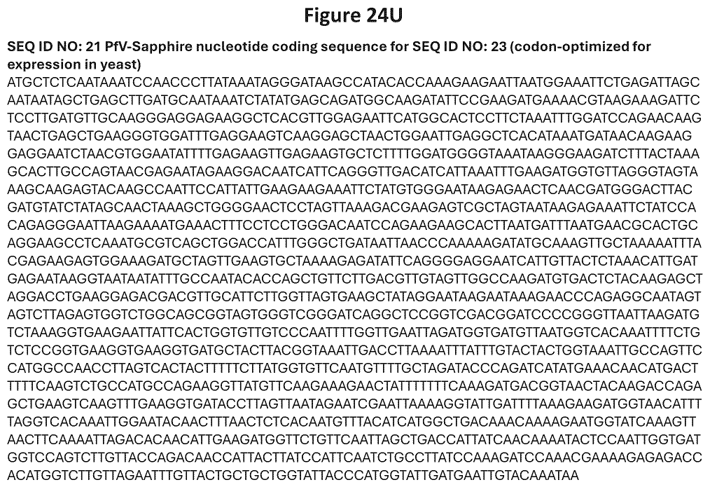

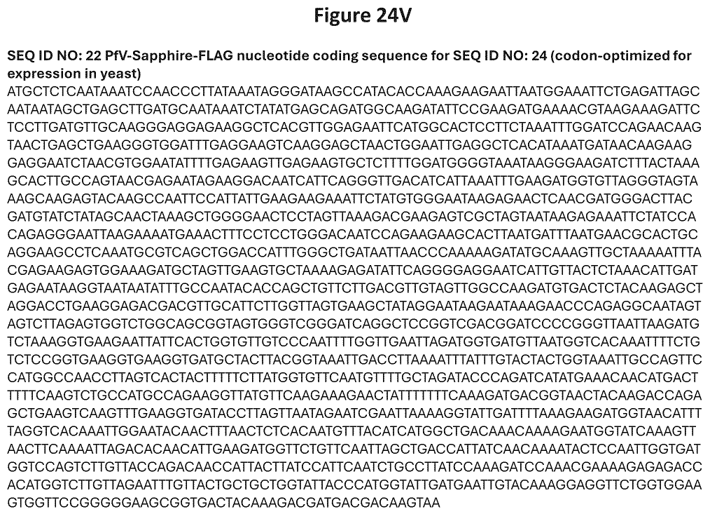

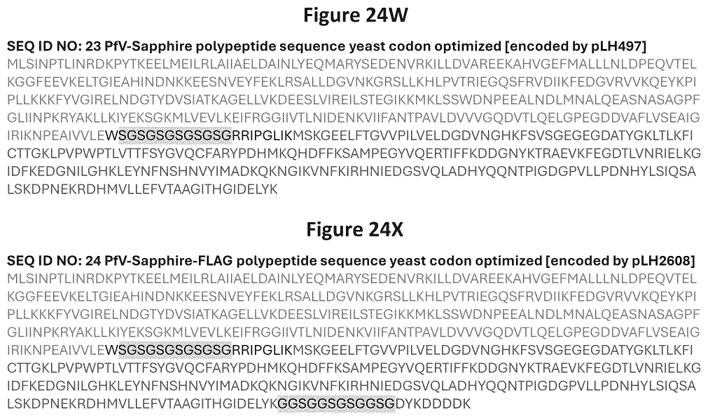

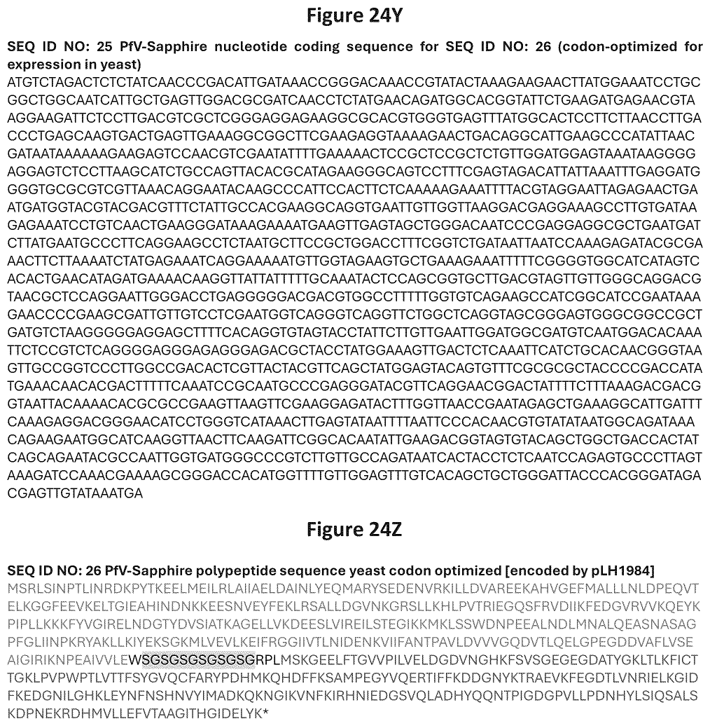

The results herein were consistent with a model in which cytoplasmic RNAs restrict mesoscale particle motion and increase elastic confinement at the 100 nm length-scale. This model predicted that decreasing the concentration of cytoplasmic RNA should increase mesoscale diffusivity. In mammalian cells, the antiviral RNase L (88,89) was induced by transfecting cells with poly (I:C), leading to widespread cytosolic RNA degradation and formation of G3BP1 protein foci (FIG. 23A) (90). Widespread reduction in cytosolic mRNA upon poly (I:C) treatment using poly (dT) FISH was confirmed, and mesoscale scale diffusivity in cells with depleted mRNA was examined (as indicated in live cells by formation of G3BP1 foci) (FIGS. 23B-23C). These cells had G3BP1 tagged with GFP, necessitating use of GEMs in a different fluorescent channel. 40 nm-GEMs were not bright enough when tagged with red-fluorescent proteins, therefore a brighter 50 nm diameter GEM nanoparticle (50 nm-GEMs) was developed based on a Quasibacillus thermotolerans encapsulin scaffold (91) that enabled use of the red fluorescent protein mScarlet. It was confirmed that 50 nm-GEMs were not sequestered within G3BP1-GFP foci (FIG. 22E). Consistent with the hypothesis herein, it was found that poly (I:C) treatment resulted in increased the diffusivity of 50 nm-GEMs (FIG. 23D, FIG. 22F). These highlighted the importance of RNA concentration as a determinant of the mesoscale physical properties of the cytoplasm in mammalian cells, and showed that an innate immune signaling pathway can change these properties.

### Example 11. Synthetic Optogenetic Condensation of RNA Increases Mesoscale Diffusivity in the Cytoplasm

To test whether RNA condensation can fluidize the mammalian cell cytoplasm independent of translation inhibition and polysome collapse, a strategy was developed to induce synthetic RNA condensates without cellular stress. Artificially assembled RNP granules containing the pumilio homology domain (PUM.HD) from the human Pumilio 1 protein were previously demonstrated to condense RNA (92,93). The artificial PUM.HD-RNP system was modified by adding the homo-oligomerization cryptochrome 2 (CRY2) domain from Arabidopsis thaliana (94,95), so that blue light could be used to control its dynamic assembly. This light-inducible condensate was named “opto-PUM” (FIG. 23E). A CRY2-mCherry construct was also made with no RNA binding domain, and it was named “opto-control” (FIG. 23E). Within minutes of blue light activation, the formation of numerous opto-PUM foci was observed, while no foci were formed in Opto-control cells (FIG. 23F). Addition of cycloheximide did not prevent Opto-PUM foci formation upon blue light activation, suggesting that induction of Opto-PUM foci did not require polysome collapse (FIG. 22G). 40 nm-GEMs were then coexpressed to quantify mesoscale diffusivity. 40 nm-GEMs did not partition into opto-PUM foci, indicating that they are reliable probes of diffusivity (FIG. 23F). A significant increase in the diffusivity of 40 nm-GEMs upon formation of Opto-PUM foci was observed (FIG. 23G). These results were consistent with the model that RNA condensation can fluidize the cytoplasm.

Ribosome density was previously shown to be a key determinant of cytoplasmic macromolecular crowding (21). However, the role of higher-order ribosome organization had not previously been addressed. Herein, it was found that assembly of ribosomes and mRNAs into polysomes strongly impacted the motion of mesoscale particles in the cytoplasm. The present cycloheximide experiments demonstrated that polysome disassembly was required for cytoplasmic fluidization in stress conditions. Upon disassembly, polysomes were converted to translationally inactive monosomes and ribosome-free mRNA. Several possible mechanisms exist to sequester and regulate this mRNA. However, it was difficult to experimentally isolate the effects of changes in polysome fractions on mesoscale physical properties. Therefore, a minimal simulation model was developed and it was found that decreasing polysome concentration was sufficient to increase mesoscale particle diffusivity. The importance of higher order ribosome organization for the physical properties of the cytoplasm can provide a general mechanism for rapid regulation as almost all stresses led to translational arrest and disassembly of polysomes.

Polysome disassembly was necessary but not sufficient for cytoplasmic fluidization; rather, it was found that mRNA condensation was also required to reduce elastic confinement and increase mesoscale diffusivity. Yeast mutants that failed to partition mRNAs into stress granules showed defects on mesoscale fluidization in several stresses, while mRNA condensation into P-bodies was most important in acute glucose starvation stress. Without wishing to be bound by theory, it is possible that these different mechanisms of mRNA condensation may be required because these stresses have distinct effects on other cytoplasmic physicochemical parameters, for example, cytosolic pH is most strongly decreased during glucose starvation (2,3,46).

It has been estimated that only 10% of cytoplasmic mRNAs partition into P-bodies and stress granules during stress (96-99). Thus, it may seem surprising that disruption of these condensates has such a pronounced effect on mesoscale movement in the cell. Without wishing to be bound by theory, it is possible that the concentration of mRNA and other polymers in the cytoplasm was close to a percolation transition, such that a small change in interaction propensity or concentration could lead to a significant change in physical properties (100,101); an analogous percolation transition is the solidification that happens over a narrow temperature change when heating an egg.

Stresses that caused polysome collapse and stress granule formation also fluidized the cytoplasm in mammalian cells, and deletion of the key stress granule scaffolds G3BP1 and G3BP2 was sufficient to prevent this physical change. Furthermore, either degrading mRNA, or partitioning mRNAs into synthetic condensates in the absence of stress both led to cytoplasmic fluidization. Thus, the phenomenon of mesoscale cytoplasmic fluidization driven by the unifying fundamental processes of polysome collapse and RNA condensation into RNP granules has been conserved over more than a billion years of eukaryotic evolution and is likely of broad relevance.

It may be valuable to further characterize the biophysical effects of P-bodies in mammalian cells, or other types of cytosolic RNP granules such as the recently described TIS granules, a large reticular meshwork that is intertwined with the endoplasmic reticulum driven by the RNA binding protein TIS11B, which has been shown to compartmentalize some mRNAs near the endoplasmic reticulum (102,103).

The dynamic biophysical changes that occur during stress are likely to have widespread impacts on biochemistry. It was proposed herein that the initial fluidization of the cytoplasm allows for rapid reorganization of the cell, enabling adaptation to the stress condition. In all stresses, proteins tend to misfold, and the creation of mesoscale membraneless organelles is an important mechanism to allow for refolding or degradation (11). Phase separation of membraneless organelles requires mesoscale subassemblies to fuse together, a process that can be inhibited by mesoscale viscoelastic networks (18,72). It was found herein that the initial fluidization of the cytoplasm was important for the formation of Q-bodies. It is possible that other mesoscale assemblies that are known to assemble during ATP depletion, such as metabolic enzyme assemblies (3,37,104,105) also require cytoplasmic fluidization for their formation.

In addition to the formation of protein aggregates, changes in membrane structure have been observed during glucose starvation in yeast. Endocytosis and exocytosis change the complement of carbon transporters (106), a process that requires efficient trafficking of vesicles of approximately 100 nm diameter. There are also large scale changes in organelle structure, including mitochondrial morphology changes (107), changes in inter-organelle connectivity, including mitochondria-ER contacts, and the nuclear-vacuole junction (10). These findings indicated that membrane reorganization within the cytoplasm is critical to ensure cell survival during long-term stresses. Therefore, fluidization of the cytoplasm may also facilitate reorganization of membrane-bound organelles.

It was demonstrated herein that the formation of P-bodies in yeast cells enabled the efficient formation of Q-bodies during glucose starvation stress. The possible biophysical and functional roles of stress granule formation can be further explored. There is growing evidence for the involvement of stress granules in human diseases. For example, cancer cells with oncogenic KRAS mutations upregulate stress granules through the production of bioactive lipid prostaglandins, leading to the inhibition of translation initiation factor eIF4A. Enhanced stress granule formation in this context has been shown to increase tumor fitness (108). These observations imply that stress granule formation confers a selective advantage to cancer cells. Without wishing to be bound by theory, it is possible that the biophysical effects of stress granule assembly could contribute to this adaptation.

On the other hand, aberrant stress granule formation appears to be cytotoxic in neurodegenerative diseases (109). The cytoplasmic pathology observed in end-stage neurodegeneration often involves the accumulation of unconventional stress granules containing mislocalized RNA-binding proteins, such as aggregated FUS (110,111) or mutated TDP43 with abnormal phosphorylation (112). Misregulation of RNA binding proteins is likely to alter the assembly kinetics of stress granules, potentially impacting the biophysical properties of the cytoplasm. The present disclosure motivated the detailed study of these properties in diseased neurons: aberrant crowding and fluidity could contribute to the proteostatic collapse that underpins all neurodegeneration. It is interesting to note that neurons typically have low levels of polysomes, and many mRNAs in neurites are translated as monosomes (113). Furthermore, mRNA is typically transported in condensates or granules in neurons (114,115). Without wishing to be bound by theory, it is possible that these adaptations could help maintain fluidity and facilitate mesoscale motion in these complex cells.

Stress granules and P bodies were first identified more than two decades ago but their functional significance has remained opaque. The present findings provided key functional insights into their essential role. Furthermore, a unifying biophysical consequence of RNA condensation into stress granules was revealed herein, establishing a new framework to investigate the role of RNA condensates in both normal physiology and disease.

### REFERENCES

1. Parry, B. R., Surovtsev, I. V., Cabeen, M. T., O'Hern, C. S., Dufresne, E. R., and Jacobs-Wagner, C. (2014). The bacterial cytoplasm has glass-like properties and is fluidized by metabolic activity. Cell 156, 183-194. PMID: 24361104.
2. Joyner, R. P., Tang, J. H., Helenius, J., Dultz, E., Brune, C., Holt, L. J., Huet, S., Müller, D. J., and Weis, K. (2016). A glucose-starvation response regulates the diffusion of macromolecules. Elife 5. 10.7554/eLife.09376.
3. Munder, M. C., Midtvedt, D., Franzmann, T., Nüske, E., Otto, O., Herbig, M., Ulbricht, E., Müller, P., Taubenberger, A., Maharana, S., et al. (2016). A pH-driven transition of the cytoplasm from a fluid-to a solid-like state promotes entry into dormancy. Preprint, 10.7554/elife.09347 10.7554/elife.09347.
4. Görner, W., Durchschlag, E., Wolf, J., Brown, E. L., Ammerer, G., Ruis, H., and Schüller, C. (2002). Acute glucose starvation activates the nuclear localization signal of a stress-specific yeast transcription factor. EMBO J. 21, 135-144.
5. Muzzey, D., Gómez-Uribe, C. A., Mettetal, J. T., and van Oudenaarden, A. (2009). A systems-level analysis of perfect adaptation in yeast osmoregulation. Cell 138, 160-171.
6. Delaunay, A., Isnard, A. D., and Toledano, M. B. (2000). H₂O₂ sensing through oxidation of the Yap1 transcription factor. EMBO J. 19, 5157-5166.
7. Triandafillou, C. G., Katanski, C. D., Dinner, A. R., and Drummond, D. A. (2020). Transient intracellular acidification regulates the core transcriptional heat shock response. Elife 9. 10.7554/eLife.54880.
8. Laidlaw, K. M. E., Bisinski, D. D., Shashkova, S., Paine, K. M., Veillon, M. A., Leake, M. C., and MacDonald, C. (2021). A glucose-starvation response governs endocytic trafficking and eisosomal retention of surface cargoes in budding yeast. J. Cell Sci. 134. 10.1242/jcs.257733.
9. Seo, A. Y., Lau, P.-W., Feliciano, D., Sengupta, P., Gros, M. A. L., Cinquin, B., Larabell, C. A., and Lippincott-Schwartz, J. (2017). AMPK and vacuole-associated Atg14p orchestrate u-lipophagy for energy production and long-term survival under glucose starvation. Elife 6. 10.7554/eLife.21690.
10. Wood, N. E., Kositangool, P., Hariri, H., Marchand, A. J., and Henne, W. M. (2020). Nutrient Signaling, Stress Response, and Inter-organelle Communication Are Non-canonical Determinants of Cell Fate. Cell Rep. 33, 108446.
11. Escusa-Toret, S., Vonk, W. I. M., and Frydman, J. (2013). Spatial sequestration of misfolded proteins by a dynamic chaperone pathway enhances cellular fitness during stress. Nat. Cell Biol. 15, 1231-1243.
12. Kaganovich, D., Kopito, R., and Frydman, J. (2008). Misfolded proteins partition between two distinct quality control compartments. Nature 454, 1088-1095.
13. Sontag, E. M., Morales-Polanco, F., Chen, J.-H., McDermott, G., Dolan, P. T., Gestaut, D., Le Gros, M. A., Larabell, C., and Frydman, J. (2022). An ESCRT-dependent pathway coordinates Nuclear and Cytoplasmic Spatial Protein Quality Control at Nuclear Vacuolar Junctions. bioRxiv, 2022.12.01.518779. 10.1101/2022.12.01.518779.
14. Sathyanarayanan, U., Musa, M., Bou Dib, P., Raimundo, N., Milosevic, I., and Krisko, A. (2020). ATP hydrolysis by yeast Hsp104 determines protein aggregate dissolution and size in vivo. Nat. Commun. 11, 5226.
15. Specht, S., Miller, S. B. M., Mogk, A., and Bukau, B. (2011). Hsp42 is required for sequestration of protein aggregates into deposition sites in Saccharomyces cerevisiae. J. Cell Biol. 195, 617-629.
16. Ekman, A. A., Chen, J.-H., Guo, J., McDermott, G., Le Gros, M. A., and Larabell, C. A. (2017). Mesoscale imaging with cryo-light and X-rays: Larger than molecular machines, smaller than a cell. Biol. Cell 109, 24-38.
17. Holt, L. J., and Delarue, M. (2023). Macromolecular crowding: Sensing without a sensor. Curr. Opin. Cell Biol. 85, 102269.
18. Shu, T., Mitra, G., Alberts, J., Viana, M. P., Levy, E. D., Hocky, G. M., and Holt, L. J. (2023). Mesoscale molecular assembly is favored by the active, crowded cytoplasm. bioRxiv. 10.1101/2023.09.19.558334.
19. Bonucci, M., Shu, T., and Holt, L. J. (2023). How it feels in a cell. Trends Cell Biol. 33, 924-938.
20. Carlini, L., Brittingham, G. P., Holt, L. J., and Kapoor, T. M. (2020). Microtubules Enhance Mesoscale Effective Diffusivity in the Crowded Metaphase Cytoplasm. Dev. Cell 54, 574-582.e4.
21. Delarue, M., Brittingham, G. P., Pfeffer, S., Surovtsev, I. V., Pinglay, S., Kennedy, K. J., Schaffer, M., Gutierrez, J. I., Sang, D., Poterewicz, G., et al. (2018). mTORC1 Controls Phase Separation and the Biophysical Properties of the Cytoplasm by Tuning Crowding. Cell 174, 338-349.e20. PMID: 29937223.
22. Advani, V. M., and Ivanov, P. (2019). Translational Control under Stress: Reshaping the Translatome. Bioessays 41, e1900009.
23. Crawford, R. A., and Pavitt, G. D. (2019). Translational regulation in response to stress in Saccharomyces cerevisiae. Yeast 36, 5-21.
24. Janapala, Y., Preiss, T., and Shirokikh, N. E. (2019). Control of Translation at the Initiation Phase During Glucose Starvation in Yeast. Int. J. Mol. Sci. 20. 10.3390/ijms20164043.
25. Ashe, M. P., De Long, S. K., and Sachs, A. B. (2000). Glucose depletion rapidly inhibits translation initiation in yeast. Mol. Biol. Cell 11, 833-848.
26. Kuhn, K. M., DeRisi, J. L., Brown, P. O., and Sarnow, P. (2001). Global and specific translational regulation in the genomic response of Saccharomyces cerevisiae to a rapid transfer from a fermentable to a nonfermentable carbon source. Mol. Cell. Biol. 21, 916-927.
27. Bresson, S., Shchepachev, V., Spanos, C., Turowski, T. W., Rappsilber, J., and Tollervey, D. (2020). Stress-Induced Translation Inhibition through Rapid Displacement of Scanning Initiation Factors. Mol. Cell 80, 470-484.e8.
28. Decker, C. J., and Parker, R. (2012). P-bodies and stress granules: possible roles in the control of translation and mRNA degradation. Cold Spring Harb. Perspect. Biol. 4, a012286.
29. Riggs, C. L., Kedersha, N., Ivanov, P., and Anderson, P. (2020). Mammalian stress granules and P bodies at a glance. J. Cell Sci. 133. 10.1242/jcs.242487.
30. Rao, B. S., and Parker, R. (2017). Numerous interactions act redundantly to assemble a tunable size of P bodies in Saccharomyces cerevisiae. Proc. Natl. Acad. Sci. U.S.A 114, E9569-E9578.
31. Jain, S., Wheeler, J. R., Walters, R. W., Agrawal, A., Barsic, A., and Parker, R. (2016). ATPase-Modulated Stress Granules Contain a Diverse Proteome and Substructure. Cell 164, 487-498.
32. Glauninger, H., Bard, J. A. M., Wong Hickernell, C. J., Airoldi, E. M., Li, W., Singer, R. H., Paul, S., Fei, J., Sosnick, T. R., Wallace, E. W. J., et al. (2024). Transcriptome-wide mRNA condensation precedes stress granule formation and excludes stress-induced transcripts. bioRxiv. 10.1101/2024.04.15.589678.
33. Desroches Altamirano, C., Kang, M.-K., Jordan, M. A., Borianne, T., Dilmen, I., Gnädig, M., von Appen, A., Honigmann, A., Franzmann, T. M., and Alberti, S. (2024). eIF4F is a thermo-sensing regulatory node in the translational heat shock response. Mol. Cell. 10.1016/j.molcel.2024.02.038.
34. Glauninger, H., Wong Hickernell, C. J., Bard, J. A. M., and Drummond, D. A. (2022). Stressful steps: Progress and challenges in understanding stress-induced mRNA condensation and accumulation in stress granules. Mol. Cell 82, 2544-2556.
35. Mason, T. G., and Weitz, D. A. (1995). Optical measurements of frequency-dependent linear viscoelastic moduli of complex fluids. Phys. Rev. Lett. 74, 1250-1253.
36. Xiang, Y., Surovtsev, I. V., Chang, Y., Govers, S. K., Parry, B. R., Liu, J., and Jacobs-Wagner, C. (2021). Interconnecting solvent quality, transcription, and chromosome folding in Escherichia coli. Cell 184, 3626-3642.e14.
37. Plante, S., Moon, K.-M., Lemieux, P., Foster, L. J., and Landry, C. R. (2023). Breaking spore dormancy in budding yeast transforms the cytoplasm and the solubility of the proteome. PLOS Biol. 21, e3002042.
38. Chambers, J. E., Zubkov, N., Kubánková, M., Nixon-Abell, J., Mela, I., Abreu, S., Schwiening, M., Lavarda, G., López-Duarte, I., Dickens, J. A., et al. (2022). Z-a1-antitrypsin polymers impose molecular filtration in the endoplasmic reticulum after undergoing phase transition to a solid state. Sci Adv 8, eabm2094.
39. Shu, T., Szórádi, T., Kidiyoor, G. R., Xie, Y., Herzog, N. L., Bazley, A., Bonucci, M., Keegan, S., Saxena, S., Ettefa, F., et al. (2022). nucGEMs probe the biophysical properties of the nucleoplasm. biorxiv. 10.1101/2021.11.18.469159.
40. Heinrich, S., Sidler, C. L., Azzalin, C. M., and Weis, K. (2017). Stem-loop RNA labeling can affect nuclear and cytoplasmic mRNA processing. RNA 23, 134-141.
41. Xie, Y., Gresham, D., and Holt, L. (2023). Increased mesoscale diffusivity in response to acute glucose starvation. bioRxiv. 10.1101/2023.01.10.523352.
42. Einstein, A. (1905). Über die von der molekularkinetischen Theorie der Wärme geforderte Bewegung von in ruhenden Flüssigkeiten suspendierten Teilchen. Annalen der Physik 322, 549-560.
43. Stokes, G. G. (1901). On the Effect of the Internal Friction of Fluids on the Motion of Pendulums. Mathematical and Physical Papers, 1-10.
44. Weber, S. C., Thompson, M. A., Moerner, W. E., Spakowitz, A. J., and Theriot, J. A. (2012). Analytical tools to distinguish the effects of localization error, confinement, and medium elasticity on the velocity autocorrelation function. Biophys. J. 102, 2443-2450.
45. Martínez-Muñoz, G. A., and Kane, P. (2017). Vacuolar and plasma membrane proton pumps collaborate to achieve cytosolic pH homeostasis in yeast. J. Biol. Chem. 292, 7743.
46. Gutierrez, J. I., Brittingham, G. P., Karadeniz, Y., Tran, K. D., Dutta, A., Holehouse, A. S., Peterson, C. L., and Holt, L. J. (2022). SWI/SNF senses carbon starvation with a pH-sensitive low-complexity sequence. Elife 11. 10.7554/eLife.70344.
47. Yaginuma, H., Kawai, S., Tabata, K. V., Tomiyama, K., Kakizuka, A., Komatsuzaki, T., Noji, H., and Imamura, H. (2014). Diversity in ATP concentrations in a single bacterial cell population revealed by quantitative single-cell imaging. Sci. Rep. 4, 6522.
48. Takaine, M., Ueno, M., Kitamura, K., Imamura, H., and Yoshida, S. (2019). Reliable imaging of ATP in living budding and fission yeast. J. Cell Sci. 132. 10.1242/jcs.230649.
49. Miesenböck, G., De Angelis, D. A., and Rothman, J. E. (1998). Visualizing secretion and synaptic transmission with pH-sensitive green fluorescent proteins. Nature 394, 192-195.
50. Boehlke, K. W., and Friesen, J. D. (1975). Cellular content of ribonucleic acid and protein in Saccharomyces cerevisiae as a function of exponential growth rate: calculation of the apparent peptide chain elongation rate. J. Bacteriol. 121, 429-433.
51. Waldron, C., Jund, R., and Lacroute, F. (1977). Evidence for a high proportion of inactive ribosomes in slow-growing yeast cells. Biochem. J 168, 409-415.
52. Miller, O. L., Jr, Hamkalo, B. A., and Thomas, C. A., Jr (1970). Visualization of bacterial genes in action. Science 169, 392-395.
53. Arava, Y., Wang, Y., Storey, J. D., Liu, C. L., Brown, P. O., and Herschlag, D. (2003). Genome-wide analysis of mRNA translation profiles in Saccharomyces cerevisiae. Proc. Natl. Acad. Sci. U.S.A 100, 3889-3894.
54. Ingolia, N. T., Ghaemmaghami, S., Newman, J. R. S., and Weissman, J. S. (2009). Genome-wide analysis in vivo of translation with nucleotide resolution using ribosome profiling. Science 324, 218-223.
55. Ben-Shem, A., Garreau de Loubresse, N., Melnikov, S., Jenner, L., Yusupova, G., and Yusupov, M. (2011). The structure of the eukaryotic ribosome at 3.0 A resolution. Science 334, 1524-1529.
56. Balagopal, V., and Parker, R. (2011). Stml modulates translation after 80S formation in Saccharomyces cerevisiae. RNA 17, 835-842.
57. Schneider-Poetsch, T., Ju, J., Eyler, D. E., Dang, Y., Bhat, S., Merrick, W. C., Green, R., Shen, B., and Liu, J. O. (2010). Inhibition of eukaryotic translation elongation by cycloheximide and lactimidomycin. Nat. Chem. Biol. 6, 209-217.
58. Xue, L., Lenz, S., Zimmermann-Kogadeeva, M., Tegunov, D., Cramer, P., Bork, P., Rappsilber, J., and Mahamid, J. (2022). Publisher Correction: Visualizing translation dynamics at atomic detail inside a bacterial cell. Nature 611, E13.
59. de Teresa-Trueba, I., Goetz, S. K., Mattausch, A., Stojanovska, F., Zimmerli, C. E., Toro-Nahuelpan, M., Cheng, D. W. C., Tollervey, F., Pape, C., Beck, M., et al. (2023). Convolutional networks for supervised mining of molecular patterns within cellular context. Nat. Methods 20, 284-294.
60. Buchan, J. R., Muhlrad, D., and Parker, R. (2008). P bodies promote stress granule assembly in Saccharomyces cerevisiae. J. Cell Biol. 183, 441-455.
61. Lui, J., Campbell, S. G., and Ashe, M. P. (2010). Inhibition of translation initiation following glucose depletion in yeast facilitates a rationalization of mRNA content. Biochem. Soc. Trans. 38, 1131-1136.
62. Stoecklin, G., and Kedersha, N. (2013). Relationship of GW/P-bodies with stress granules. Adv. Exp. Med. Biol. 768, 197-211.
63. Teixeira, D., Sheth, U., Valencia-Sanchez, M. A., Brengues, M., and Parker, R. (2005). Processing bodies require RNA for assembly and contain nontranslating mRNAs. RNA 11, 371-382.
64. Decker, C. J., Teixeira, D., and Parker, R. (2007). Edc3p and a glutamine/asparagine-rich domain of Lsm4p function in processing body assembly in Saccharomyces cerevisiae. J. Cell Biol. 179, 437-449.
65. Xing, W., Muhlrad, D., Parker, R., and Rosen, M. K. (2020). A quantitative inventory of yeast P body proteins reveals principles of composition and specificity. Elife 9. 10.7554/eLife.56525.
66. Currie, S. L., Xing, W., Muhlrad, D., Decker, C. J., Parker, R., and Rosen, M. K. (2023). Quantitative reconstitution of yeast RNA processing bodies. Proc. Natl. Acad. Sci. U.S.A. 120, e2214064120.
67. Riback, J. A., Katanski, C. A., Kear-Scott, J. L., Pilipenko, E. V., Sosnick, T. R., and Drummond, D. A. (2017). How evolution tunes stress-triggered protein phase separation to promote cell fitness during stress. Biophys. J. 112, 5a.
68. Holstege, F. C., Jennings, E. G., Wyrick, J. J., Lee, T. I., Hengartner, C. J., Green, M. R., Golub, T. R., Lander, E. S., and Young, R. A. (1998). Dissecting the regulatory circuitry of a eukaryotic genome. Cell 95, 717-728.
69. Zenklusen, D., Larson, D. R., and Singer, R. H. (2008). Single-RNA counting reveals alternative modes of gene expression in yeast. Nat. Struct. Mol. Biol. 15, 1263-1271.
70. Iserman, C., Desroches Altamirano, C., Jegers, C., Friedrich, U., Zarin, T., Fritsch, A. W., Mittasch, M., Domingues, A., Hersemann, L., Jahnel, M., et al. (2020). Condensation of Dedlp Promotes a Translational Switch from Housekeeping to Stress Protein Production. Cell 181, 818-831.e19.
71. Kroschwald, S., Munder, M. C., Maharana, S., Franzmann, T. M., Richter, D., Ruer, M., Hyman, A. A., and Alberti, S. (2018). Different Material States of Publ Condensates Define Distinct Modes of Stress Adaptation and Recovery. Cell Rep. 23, 3327-3339.
72. Lee, D. S. W., Wingreen, N. S., and Brangwynne, C. P. (2021). Chromatin mechanics dictates subdiffusion and coarsening dynamics of embedded condensates. Nat. Phys. 17, 531-538.
73. Stein, K. C., and Frydman, J. (2019). The stop-and-go traffic regulating protein biogenesis: How translation kinetics controls proteostasis. J. Biol. Chem. 294, 2076-2084.
74. Takaine, M., Imamura, H., and Yoshida, S. (2022). High and stable ATP levels prevent aberrant intracellular protein aggregation in yeast. Elife 11. 10.7554/eLife.67659.
75. Brewster, J. L., de Valoir, T., Dwyer, N. D., Winter, E., and Gustin, M. C. (1993). An osmosensing signal transduction pathway in yeast. Science 259, 1760-1763.
76. Hohmann, S., Krantz, M., and Nordlander, B. (2007). Yeast osmoregulation. Methods Enzymol. 428, 29-45.
77. Lavalée, M., Curdy, N., Laurent, C., Fournié, J.-J., and Franchini, D.-M. (2021). Cancer cell adaptability: turning ribonucleoprotein granules into targets. Trends Cancer Res. 7, 902-915.
78. Ash, P. E. A., Vanderweyde, T. E., Youmans, K. L., Apicco, D. J., and Wolozin, B. (2014). Pathological stress granules in Alzheimer's disease. Preprint, 10.1016/j.brainres.2014.05.052 10.1016/j.brainres.2014.05.052.
79. Yang, P., Mathieu, C., Kolaitis, R.-M., Zhang, P., Messing, J., Yurtsever, U., Yang, Z., Wu, J., Li, Y., Pan, Q., et al. (2020). G3BP1 Is a Tunable Switch that Triggers Phase Separation to Assemble Stress Granules. Cell 181, 325-345.e28.
80. Guillén-Boixet, J., Kopach, A., Holehouse, A. S., Wittmann, S., Jahnel, M., Schlüβler, R., Kim, K., Trussina, I. R. E. A., Wang, J., Mateju, D., et al. (2020). RNA-Induced Conformational Switching and Clustering of G3BP Drive Stress Granule Assembly by Condensation. Cell 181, 346-361.e17.
81. Broering, T. J. (2002). Initial Characterization of Reovirus Nonstructural Protein NS and Its Activities (University of Wisconsin—Madison).
82. Kudla, M., and Karginov, F. V. (2016). Measuring mRNA Translation by Polysome Profiling. Methods Mol. Biol. 1421, 127-135.
83. Baliga, B. S., Cohen, S. A., and Munro, H. N. (1970). Effect of cycloheximide on the reaction of puromycin with polysome-bound peptidyl-tRNA. FEBS Lett. 8, 249-252.
84. Kommaraju, S. S., Aulicino, J., Gobbooru, S., Li, J., Zhu, M., Romo, D., and Low, W.-K. (2020). Investigation of the mechanism of action of a potent pateamine A analog, des-methyl, des-amino pateamine A (DMDAPatA). Biochem. Cell Biol. 98, 502-510.
85. Dang, Y., Kedersha, N., Low, W.-K., Romo, D., Gorospe, M., Kaufman, R., Anderson, P., and Liu, J. O. (2006). Eukaryotic initiation factor 2alpha-independent pathway of stress granule induction by the natural product pateamine A. J. Biol. Chem. 281, 32870-32878.
86. Anand, A. A., and Walter, P. (2020). Structural insights into ISRIB, a memory-enhancing inhibitor of the integrated stress response. FEBS J. 287, 239-245.
87. Sidrauski, C., McGeachy, A. M., Ingolia, N. T., and Walter, P. (2015). The small molecule ISRIB reverses the effects of eIF2a phosphorylation on translation and stress granule assembly. Elife 4. 10.7554/eLife.05033.
88. Rath, S., Prangley, E., Donovan, J., Demarest, K., Wingreen, N. S., Meir, Y., and Korennykh, A. (2019). Concerted 2-5A-Mediated mRNA Decay and Transcription Reprogram Protein Synthesis in the dsRNA Response. Mol. Cell 75, 1218-1228.e6.
89. Burke, J. M., Moon, S. L., Matheny, T., and Parker, R. (2019). RNase L Reprograms Translation by Widespread mRNA Turnover Escaped by Antiviral mRNAs. Mol. Cell 75, 1203-1217.e5.
90. Decker, C. J., Burke, J. M., Mulvaney, P. K., and Parker, R. (2022). RNA is required for the integrity of multiple nuclear and cytoplasmic membrane-less RNP granules. EMBO J. 41, e110137.
91. Giessen, T. W., Orlando, B. J., Verdegaal, A. A., Chambers, M. G., Gardener, J., Bell, D. C., Birrane, G., Liao, M., and Silver, P. A. (2019). Large protein organelles form a new iron sequestration system with high storage capacity. Elife 8. 10.7554/eLife.46070. PMID: 31282860.
92. Garcia-Jove Navarro, M., Kashida, S., Chouaib, R., Souquere, S., Pierron, G., Weil, D., and Gueroui, Z. (2019). RNA is a critical element for the sizing and the composition of phase-separated RNA-protein condensates. Nat. Commun. 10, 1-13.
93. Cochard, A., Garcia-Jove Navarro, M., Piroska, L., Kashida, S., Kress, M., Weil, D., and Gueroui, Z. (2022). RNA at the surface of phase-separated condensates impacts their size and number. Biophys. J. 121, 1675-1690.
94. Che, D. L., Duan, L., Zhang, K., and Cui, B. (2015). The Dual Characteristics of Light-Induced Cryptochrome 2, Homo-oligomerization and Heterodimerization, for Optogenetic Manipulation in Mammalian Cells. ACS Synth. Biol. 4, 1124-1135.
95. Bugaj, L. J., Choksi, A. T., Mesuda, C. K., Kane, R. S., and Schaffer, D. V. (2013). Optogenetic protein clustering and signaling activation in mammalian cells. Nat. Methods 10, 249-252.
96. Wang, C., Schmich, F., Srivatsa, S., Weidner, J., Beerenwinkel, N., and Spang, A. (2018). Correction: Context-dependent deposition and regulation of mRNAs in P-bodies. Elife 7. 10.7554/eLife.41300.
97. Khong, A., Matheny, T., Jain, S., Mitchell, S. F., Wheeler, J. R., and Parker, R. (2017). The Stress Granule Transcriptome Reveals Principles of mRNA Accumulation in Stress Granules. Mol. Cell 68, 808-820.e5.
98. Namkoong, S., Ho, A., Woo, Y. M., Kwak, H., and Lee, J. H. (2018). Systematic Characterization of Stress-Induced RNA Granulation. Mol. Cell 70, 175-187.e8.
99. Matheny, T., Rao, B. S., and Parker, R. (2019). Transcriptome-Wide Comparison of Stress Granules and P-Bodies Reveals that Translation Plays a Major Role in RNA Partitioning. Mol. Cell. Biol. 39. 10.1128/MCB.00313-19.
100. Kar, M., Dar, F., Welsh, T. J., Vogel, L. T., Kühnemuth, R., Majumdar, A., Krainer, G., Franzmann, T. M., Alberti, S., Seidel, C. A. M., et al. (2022). Phase-separating RNA-binding proteins form heterogeneous distributions of clusters in subsaturated solutions. Proc. Natl. Acad. Sci. U.S.A 119, e2202222119.
101. Mittag, T., and Pappu, R. V. (2022). A conceptual framework for understanding phase separation and addressing open questions and challenges. Mol. Cell 82, 2201-2214.
102. Ma, W., and Mayr, C. (2018). A Membraneless Organelle Associated with the Endoplasmic Reticulum Enables 3′UTR-Mediated Protein-Protein Interactions. Cell 175, 1492-1506.e19.
103. Horste, E. L., Fansler, M. M., Cai, T., Chen, X., Mitschka, S., Zhen, G., Lee, F. C. Y., Ule, J., and Mayr, C. (2023). Subcytoplasmic location of translation controls protein output. Mol. Cell 83, 4509-4523.e11.
104. Marini, G., Nüske, E., Leng, W., Alberti, S., and Pigino, G. (2020). Reorganization of budding yeast cytoplasm upon energy depletion. Mol. Biol. Cell 31, 1232-1245.
105. Petrovska, I., Nüske, E., Munder, M. C., Kulasegaran, G., Malinovska, L., Kroschwald, S., Richter, D., Fahmy, K., Gibson, K., Verbavatz, J.-M., et al. (2014). Filament formation by metabolic enzymes is a specific adaptation to an advanced state of cellular starvation. Elife 3. 10.7554/eLife.02409.
106. Feyder, S., De Craene, J.-O., Bar, S., Bertazzi, D. L., and Friant, S. (2015). Membrane trafficking in the yeast Saccharomyces cerevisiae model. Int. J. Mol. Sci. 16, 1509-1525.
107. Laporte, D., Gouleme, L., Jimenez, L., Khemiri, I., and Sagot, I. (2018). Mitochondria reorganization upon proliferation arrest predicts individual yeast cell fate. Elife 7. 10.7554/eLife.35685.
108. Grabocka, E., and Bar-Sagi, D. (2016). Mutant KRAS Enhances Tumor Cell Fitness by Upregulating Stress Granules. Cell 167, 1803-1813.e12.
109. Zhang, P., Fan, B., Yang, P., Temirov, J., Messing, J., Kim, H. J., and Taylor, J. P. (2019). Chronic optogenetic induction of stress granules is cytotoxic and reveals the evolution of ALS-FTD pathology. Elife 8. 10.7554/eLife.39578.
110. Dormann, D., Rodde, R., Edbauer, D., Bentmann, E., Fischer, I., Hruscha, A., Than, M. E., Mackenzie, I. R. A., Capell, A., Schmid, B., et al. (2010). ALS-associated fused in sarcoma (FUS) mutations disrupt Transportin-mediated nuclear import. EMBO J. 29, 2841-2857.
111. Deng, H., Gao, K., and Jankovic, J. (2014). The role of FUS gene variants in neurodegenerative diseases. Nat. Rev. Neurol. 10, 337-348.
112. Neumann, M., Kwong, L. K., Lee, E. B., Kremmer, E., Flatley, A., Xu, Y., Forman, M. S., Troost, D., Kretzschmar, H. A., Trojanowski, J. Q., et al. (2009). Phosphorylation of S409/410 of TDP-43 is a consistent feature in all sporadic and familial forms of TDP-43 proteinopathies. Acta Neuropathol. 117, 137-149.
113. Biever, A., Glock, C., Tushev, G., Ciirdaeva, E., Dalmay, T., Langer, J. D., and Schuman, E. M. (2020). Monosomes actively translate synaptic mRNAs in neuronal processes. Science 367. 10.1126/science.aay4991.
114. Knowles, R. B., Sabry, J. H., Martone, M. E., Deerinck, T. J., Ellisman, M. H., Bassell, G. J., and Kosik, K. S. (1996). Translocation of RNA granules in living neurons. J. Neurosci. 16, 7812-7820.
115. Liao, Y.-C., Fernandopulle, M. S., Wang, G., Choi, H., Hao, L., Drerup, C. M., Patel, R., Qamar, S., Nixon-Abell, J., Shen, Y., et al. (2019). RNA Granules Hitchhike on Lysosomes for Long-Distance Transport, Using Annexin All as a Molecular Tether. Cell 179, 147-164.e20.
116. Bharat, T. A. M., and Scheres, S. H. W. (2016). Resolving macromolecular structures from electron cryo-tomography data using subtomogram averaging in RELION. Nat. Protoc. 11, 2054-2065.
117. Riback, J. A., Katanski, C. D., Kear-Scott, J. L., Pilipenko, E. V., Rojek, A. E., Sosnick, T. R., and Drummond, D. A. (2017). Stress-Triggered Phase Separation Is an Adaptive, Evolutionarily Tuned Response. Cell 168, 1028-1040.e19.
118. Sbalzarini, I. F., and Koumoutsakos, P. (2005). Feature point tracking and trajectory analysis for video imaging in cell biology. J. Struct. Biol. 151, 182-195. PMID: 16043363.
119. Takaine, M. (2019). QUEEN-based Spatiotemporal ATP Imaging in Budding and Fission Yeast. Bio Protoc 9, e3320.
120. Chan, Y.-H. M., and Marshall, W. F. (2014). Organelle size scaling of the budding yeast vacuole is tuned by membrane trafficking rates. Biophys. J. 106, 1986-1996.
121. Villa, E., Schaffer, M., Plitzko, J. M., and Baumeister, W. (2013). Opening windows into the cell: focused-ion-beam milling for cryo-electron tomography. Curr. Opin. Struct. Biol. 23, 771-777.
122. Schaffer, M., Engel, B. D., Laugks, T., Mahamid, J., Plitzko, J. M., and Baumeister, W. (2015). Cryo-focused Ion Beam Sample Preparation for Imaging Vitreous Cells by Cryo-electron Tomography. Bio Protoc 5. 10.21769/bioprotoc.1575.
123. Schaffer, M., Mahamid, J., Engel, B. D., Laugks, T., Baumeister, W., and Plitzko, J. M. (2017). Optimized cryo-focused ion beam sample preparation aimed at in situ structural studies of membrane proteins. J. Struct. Biol. 197, 73-82.
124. Klumpe, S., Fung, H. K., Goetz, S. K., Zagoriy, I., Hampoelz, B., Zhang, X., Erdmann, P. S., Baumbach, J., Müller, C. W., Beck, M., et al. (2021). A modular platform for automated cryo-FIB workflows. Elife 10. 10.7554/eLife.70506.
125. Mastronarde, D. N. (2005). Automated electron microscope tomography using robust prediction of specimen movements. J. Struct. Biol. 152, 36-51.
126. Hagen, W. J. H., Wan, W., and Briggs, J. A. G. (2017). Implementation of a cryo-electron tomography tilt-scheme optimized for high resolution subtomogram averaging. J. Struct. Biol. 197, 191-198.
127. Tegunov, D., and Cramer, P. (2019). Real-time cryo-electron microscopy data preprocessing with Warp. Nat. Methods 16, 1146-1152.
128. Zheng, S., Wolff, G., Greenan, G., Chen, Z., Faas, F. G. A., Bárcena, M., Koster, A. J., Cheng, Y., and Agard, D. A. (2022). AreTomo: An integrated software package for automated marker-free, motion-corrected cryo-electron tomographic alignment and reconstruction. J Struct Biol X 6, 100068.
129. Tang, G., Peng, L., Baldwin, P. R., Mann, D. S., Jiang, W., Rees, I., and Ludtke, S. J. (2007). EMAN2: an extensible image processing suite for electron microscopy. J. Struct. Biol. 157, 38-46.
130. Kimanius, D., Dong, L., Sharov, G., Nakane, T., and Scheres, S. H. W. (2021). New tools for automated cryo-EM single-particle analysis in RELION-4.0. Biochem. J 478, 4169-4185.
131. Tegunov, D., Xue, L., Dienemann, C., Cramer, P., and Mahamid, J. (2021). Multi-particle cryo-EM refinement with M visualizes ribosome-antibiotic complex at 3.5 Å in cells. Nat. Methods 18, 186-193.
132. Pettersen, E. F., Goddard, T. D., Huang, C. C., Meng, E. C., Couch, G. S., Croll, T. I., Morris, J. H., and Ferrin, T. E. (2021). UCSF ChimeraX: Structure visualization for researchers, educators, and developers. Protein Sci. 30, 70-82.
133. Weeks, J. D., Chandler, D., and Andersen, H. C. (1971). Role of repulsive forces in determining the equilibrium structure of simple liquids. J. Chem. Phys. 54, 5237-5247. 134. Frenkel, D., and Smit, B. (2023). Understanding Molecular Simulation: From Algorithms to Applications (Elsevier).
135. Anderson, J. A., Glaser, J., and Glotzer, S. C. (2020). HOOMD-blue: A Python package for high-performance molecular dynamics and hard particle Monte Carlo simulations. Comput. Mater. Sci. 173, 109363.
136. Stukowski, A. (2009). Visualization and analysis of atomistic simulation data with OVITO—the Open Visualization Tool. Modell. Simul. Mater. Sci. Eng. 18, 015012.
137. Botman, D., de Groot, D. H., Schmidt, P., Goedhart, J., and Teusink, B. (2019). In vivo characterisation of fluorescent proteins in budding yeast. Sci. Rep. 9, 2234.
138. Hentges, P., Van Driessche, B., Tafforeau, L., Vandenhaute, J., and Carr, A. M. (2005). Three novel antibiotic marker cassettes for gene disruption and marker switching in Schizosaccharomyces pombe. Yeast 22, 1013-1019.
139. Arnold J Boersma, Inge S Zuhorn, and Bert Poolman. A sensor for quantification of macromolecular crowding in living cells. Nature Methods, 12:227-229, 2015. PMID: 25643150.
140. Lina Carlini, Gregory P. Brittingham, Liam J. Holt, and Tarun M. Kapoor. Microtubules enhance mesoscale effective diffusivity in the crowded metaphase cytoplasm. Developmental Cell, 54 (5): 574-582.E4, 2020. PMID: 32818469.
141. S. Keegan, L. J. Holt, and D. Fenyö. Gemspa: a napari plugin for analysis of single particle tracking data. bioRxiv, 2023.
142. B. R. Parry, I. V. Surovtsev, M. T. Cabeen, C. S. O'Hern, E. R. Dufresne, and C. Jacobs-Wagner. The bacterial cytoplasm has glass-like properties and is fluidized by metabolic activity. Cell, 156 (1-2): 183-194, 2013.
143. Ivo F Sbalzarini and Petros Koumoutsakos. Feature point tracking and trajectory analysis for video imaging in cell biology. Journal of Structural Biology, 151 (2): 182-195, 2005. 144. C. Stringer, T. Wang, M. Michaelos, and et al. Cellpose: a generalist algorithm for cellular segmentation. Nat Methods, 18 (1): 100-106, 2021. PMID: 33318659.
145. S. C. Weber, M. A. Thompson, W. E. Moerner, A. J. Spakowitz, and J. A. Theriot. Analytical tools to distinguish the effects of localization error, confinement, and medium elasticity on the velocity autocorrelation function. Biophysical Journal, 102 (11): 2443-2450, 2012. PMID: 22713559.
146. James A. Dix and A. S. Verkman. Crowding effects on diffusion in solutions and cells. Annual Review of Biophysics, 37 (0): 247-263, 2008. PMID: 18573081.
147. A. Einstein. Annalen der physik. 17:549-560, 1905.
148. S. Alberti and A. A. Hyman, Biomolecular condensates at the nexus of cellular stress, protein aggregation disease, and ageing. Nat Rev Mol Cell Biol, 22 (3): 196-213, 2021. PMID: 33510441.
149. J. Spitzer and B. Poolman, How crowded is the prokaryotic cytoplasm? FEBS Lett., 587:2094-2098, 2013. PMID: 23735698.
150. J. B. Woodruff et al., The centrosome is a selective condensate that nucleates microtubules by concentrating tubulin. Cell, 169:1066-1077, 2017. PMID: 28575670.
151. S. B. Zimmerman and S. O. Trach, Estimation of macromolecule concentrations and excluded volume effects for the cytoplasm of Escherichia coli. J. Mol. Biol., 222:599-620, 1991. PMID: 1748995.

152. R. John Ellis, A characteristic of the interiors of all cells is the high total concentration of macromolecules they contain. Trends in Biochemical Sciences, 26:597, 2001

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. An isolated polynucleotide encoding a fusion polypeptide comprising an encapsulin domain from Quasibacillus thermotolerans (“QtE”) and a fluorescent tag, wherein the fluorescent tag is located to the C-terminus of the QtE.

2. The polynucleotide of claim 1, wherein QtE comprises an amino acid sequence SEQ ID NO: 1 or a functional fragment thereof or a sequence that has at least 90% amino acid sequence identity thereto.

3. The polynucleotide of claim 2, wherein QtE comprises the amino acid sequence SEQ ID NO: 1.

4. The polynucleotide of claim 3, wherein QtE consists of the amino acid sequence SEQ ID NO: 1.

5. The polynucleotide of any one of claims 1-4, wherein the fluorescent tag is selected from red fluorescent protein mScarlet, green fluorescent protein (GFP), green fluorescent protein variant Sapphire, self-labeling HaloTag, and functional fragments and derivatives thereof.

6. The polynucleotide of claim 5, wherein the fluorescent tag is Sapphire which comprises an amino acid sequence SEQ ID NO: 3.

7. The polynucleotide of claim 6, wherein Sapphire consists of the amino acid sequence SEQ ID NO: 3.

8. The polynucleotide of claim 5, wherein the fluorescent tag is GFP which comprises an amino acid sequence SEQ ID NO: 5.

9. The polynucleotide of claim 8, wherein GFP consists of the amino acid sequence SEQ ID NO: 5.

10. The polynucleotide of claim 5, wherein the fluorescent tag is HaloTag which comprises an amino acid sequence SEQ ID NO: 8.

11. The polynucleotide of claim 10, wherein HaloTag consists of the amino acid sequence SEQ ID NO: 8.

12. The polynucleotide of claim 5, wherein the fluorescent tag is mScarlet which comprises an amino acid sequence SEQ ID NO: 10.

13. The polynucleotide of claim 12, wherein mScarlet consists of the amino acid sequence SEQ ID NO: 10.

14. The polynucleotide of any one of claims 1-13, wherein the fluorescent tag is attached to the C-terminus of the QtE via a linker.

15. The polynucleotide of claim 14, wherein the linker is a GS-containing linker.

16. The polynucleotide of claim 15, wherein the linker comprises an amino acid sequence SGSGSGSGSGSG (SEQ ID NO: 2).

17. The polynucleotide of claim 15, wherein the linker comprises an amino acid sequence GGSGGSGSGGSG (SEQ ID NO: 4).

18. The polynucleotide of any one of claims 1-17, wherein the fusion polypeptide further comprises a purification tag.

19. The polynucleotide of claim 18, wherein the purification tag is located to the C-terminus of the fluorescent tag.

20. The polynucleotide of claim 18 or claim 19, wherein the purification tag is a FLAG tag.

21. The polynucleotide of claim 20, wherein the FLAG tag comprises an amino acid sequence SEQ ID NO: 6.

22. The polynucleotide of any one of claims 18-21, wherein the purification tag is attached to the C-terminus of the fluorescent tag via a linker.

23. The polynucleotide of claim 22, wherein the linker is a GS-containing linker.

24. The polynucleotide of claim 23, wherein the linker comprises an amino acid sequence SGSGSGSGSGSG (SEQ ID NO: 2).

25. The polynucleotide of claim 23, wherein the linker comprises an amino acid sequence GGSGGSGSGGSG (SEQ ID NO: 4).

26. The polynucleotide of any one of claims 1-25, wherein the fusion polypeptide further comprises a sequence from the PopZ protein from Caulobacter crescentus, wherein said PopZ sequence is located to the N terminus of QtE and comprises the C-terminal helical oligomerization domain of PopZ or a functional fragment thereof or a sequence that has at least 90% amino acid sequence identity thereto.

27. The polynucleotide of claim 26, wherein the sequence from the PopZ protein comprises an amino acid sequence SEQ ID NO: 9.

28. The polynucleotide of claim 27, wherein the sequence from the PopZ protein consists of the amino acid sequence SEQ ID NO: 9.

29. The polynucleotide of any one of claims 1-28, wherein upon its expression in a host cell said fusion polypeptide forms genetically encoded multimeric nanoparticles (GEMs) having a diameter of about 50 nm.

30. The polynucleotide of claim 29, wherein upon its expression in a host cell said fusion polypeptide forms GEMs having a diameter of 45-55 nm.

31. The polynucleotide of claim 30, wherein upon its expression in a host cell said fusion polypeptide forms GEMs having a diameter of 47-53 nm.

32. The polynucleotide of claim 31, wherein upon its expression in a host cell said fusion polypeptide forms GEMs having a diameter of 48-52 nm.

33. The polynucleotide of claim 32, wherein upon its expression in a host cell said fusion polypeptide forms GEMs having a diameter of 51 nm.

34. The polynucleotide of any one of claims 29-33, wherein the host cell is a yeast cell.

35. The polynucleotide of any one of claims 29-33, wherein the host cell is a mammalian cell.

36. The polynucleotide of claim 1, wherein the fusion polypeptide comprises the amino acid sequence SEQ ID NO: 11.

37. The polynucleotide of claim 36, wherein the polynucleotide comprises the nucleotide sequence SEQ ID NO: 17.

38. The polynucleotide of claim 1, wherein the fusion polypeptide comprises the amino acid sequence SEQ ID NO: 12.

39. The polynucleotide of claim 38, wherein the polynucleotide comprises the nucleotide sequence SEQ ID NO: 18.

40. The polynucleotide of claim 1, wherein the fusion polypeptide comprises the amino acid sequence SEQ ID NO: 13.

41. The polynucleotide of claim 40, wherein the polynucleotide comprises the nucleotide sequence SEQ ID NO: 19.

42. The polynucleotide of claim 1, wherein the fusion polypeptide comprises the amino acid sequence SEQ ID NO: 14.

43. The polynucleotide of claim 42, wherein the polynucleotide comprises the nucleotide sequence SEQ ID NO: 16.

44. The polynucleotide of claim 1, wherein the fusion polypeptide comprises the amino acid sequence SEQ ID NO: 15.

45. The polynucleotide of claim 44, wherein the polynucleotide comprises the nucleotide sequence SEQ ID NO: 20.

46. The polynucleotide of claim 1, wherein the fusion polypeptide comprises the amino acid sequence SEQ ID NO: 23.

47. The polynucleotide of claim 46, wherein the polynucleotide comprises the nucleotide sequence SEQ ID NO: 21.

48. The polynucleotide of claim 1, wherein the fusion polypeptide comprises the amino acid sequence SEQ ID NO: 24.

49. The polynucleotide of claim 48, wherein the polynucleotide comprises the nucleotide sequence SEQ ID NO: 22.

50. The polynucleotide of claim 1, wherein the fusion polypeptide comprises the amino acid sequence SEQ ID NO: 26.

51. The polynucleotide of claim 50, wherein the polynucleotide comprises the nucleotide sequence SEQ ID NO: 25.

52. A fusion polypeptide encoded by the polynucleotide of any one of claims 1-51.

53. A genetically encoded multimeric nanoparticle (GEM) comprising the fusion polypeptide of claim 52.

54. The GEM of claim 53, wherein said GEM has a diameter of about 50 nm.

55. The GEM of claim 54, wherein said GEM has a diameter of 45-55 nm.

56. The GEM of claim 55, wherein said GEM has a diameter of 47-53 nm.

57. The GEM of claim 56, wherein said GEM has a diameter of 48-52 nm.

58. The GEM of claim 57, wherein said GEM has a diameter of 51 nm.

59. A vector comprising the polynucleotide of any one of claims 1-51.

60. The vector of claim 59, wherein said vector is a mammalian expression vector.

61. The vector of claim 59 or claim 60, wherein the vector is a viral vector.

62. The vector of claim 61, wherein the viral vector is a lentiviral vector.

63. The vector of any one of claims 59-62, wherein the polynucleotide encoding the fusion polypeptide is operably linked to a mammalian Ubiquitin C (UBC) promoter.

64. The vector of claim 59, wherein said vector is a yeast expression vector.

65. The vector of claim 64, wherein said yeast expression vector is a yeast integration expression vector.

66. The vector of claim 59, 64 or 65, wherein the polynucleotide encoding the fusion polypeptide is operably linked to a yeast transcription factor (INO4) promoter or a yeast histidine synthesis (His3) promoter.

67. A host cell comprising the polynucleotide of any one of claims 1-51 or the vector of any one of claims 59-66.

68. A method of producing genetically encoded multimeric nanoparticles (GEMs) comprising:
a) incubating the host cell of claim 67 under conditions when the fusion polypeptide is produced and assembles into GEMs, and
b) isolating said GEMs.

69. A method for probing a biophysical property of a cell, said method comprising transforming, transfecting, or transducing said cell with the polynucleotide of any one of claims 1-51 or the vector of any one of claims 59-66 and detecting the fluorescent tag.

70. The method of claim 69, wherein the biophysical property is selected from cytoplasmic fluidity, effective diffusivity, anomalous diffusion exponent, viscoelastic/mechanical state, cytoplasmic crowding, spatial heterogeneity, confinement/caging, non-equilibrium/active fluctuation, phase separation and condensates, and any combination thereof.

71. The method of claim 69 or claim 70, wherein a change in the biophysical property results in a biophysical consequence.

72. The method of claim 71, wherein the biophysical consequence is selected from ATP depletion or metabolic collapse, pH changes, glucose starvation, a change in ribosome concentration and/or polysome disassembly, formation of RNA condensates (P-bodies, stress granules, RNP granules), free RNA abundance, assembly of Q-bodies, and any combination thereof.
